(19) United States
(12) Patent Application Publication
(10) Pub. No.: US 2014/0206577 A1
(43) Pub. Date: Jul. 24, 2014

(54) COMPOSITIONS AND METHODS FOR MOLECULAR BIOLOGY

(71) Applicant: LIFE TECHNOLOGIES CORPORATION, Carlsbad, CA (US)

(72) Inventors: Devon YOUNG, Fredericksburg, VA (US); James HARTLEY, Frederick, MD (US); Alice YOUNG, Gaithersburg, MD (US)

(73) Assignee: LIFE TECHNOLOGIES CORPORATION, Carlsbad, CA (US)

(21) Appl. No.: 14/066,574

(22) Filed: Oct. 29, 2013

Related U.S. Application Data
(63) Continuation of application No. 13/016,838, filed on Jan. 28, 2011, now abandoned.
Application No. 13/016,838 is a continuation of application No. 11/106,715, filed on Apr. 15, 2005, now abandoned.
Application No. 11/106,715 is a continuation of application No. 10/633,690, filed on Aug. 5, 2003, now abandoned.
Application No. 10/633,690 is a continuation-in-part of application No. 10/067,543, filed on Feb. 7, 2002, now abandoned.
(60) Provisional application No. 60/400,704, filed on Aug. 5, 2002.
(60) Provisional application No. 60/403,095, filed on Aug. 14, 2002.
(60) Provisional application No. 60/266,846, filed on Feb. 7, 2001.

(51) Int. Cl. C12N 15/63; C12Q 1/68

(52) U.S. Cl. CPC C12N 15/63; C12Q 1/6834
USPC 506/16; 435/320.1; 530/350; 435/174; 506/18

(57) ABSTRACT

The present invention provides materials and methods for the utilization of the specific interaction of replication termination sequences with their binding proteins in molecular biology applications.

### CROSS-REFERENCE TO RELATED APPLICATIONS

The present application is a continuation of U.S. application Ser. No. 10/633,690, filed Aug. 5, 2003, which claims the benefit of the filing dates of U.S. Provisional Application No. 60/400,704, filed Aug. 5, 2002, and U.S. Provisional Application No. 60/403,095, filed Aug. 14, 2002, the disclosures of which applications are incorporated by reference herein in their entireties. The present application is also a continuation-in-part of U.S. application Ser. No. 10/067,543, filed Feb. 7, 2002, which claims the benefit of the filing date of U.S. Provisional Application No. 60/266,846, filed Feb. 7, 2001, the disclosures of which applications are incorporated by reference herein in their entireties.

### BACKGROUND OF THE INVENTION

1. Field of the Invention

The present invention is in the field of molecular biology. The invention is related generally to polynucleotides and polypeptides that interact specifically with the polynucleotides, and methods for their use. Specifically, the invention provides polynucleotides, termination sequences, and nucleic acid binding proteins that bind to termination sequences and methods of using one or more of these for cloning, for selecting a nucleic acid of interest, for purifying a polynucleotide of interest, for producing single-stranded DNA, for juxtaposing at least two sites of a polynucleotide, for maintaining topology of a nucleic acid molecule, for detecting target sequences and other biomolecules, for immobilizing polynucleotides onto a support, among other uses. The invention also relates to fragments or derivatives of these polynucleotides and polypeptides, and to vectors comprising such polynucleotides or encoding such polypeptides as well as host cells comprising such vectors, and fragments, or derivatives thereof. The invention also concerns kits comprising the polynucleotides, polypeptides and/or compositions of the invention.

2. Related Art

In bacterial systems, replication of genomes and plasmids begins at a specific site on the genome or plasmid termed the origin of replication (ori). Replication is initiated at the origin of replication and proceeds either unidirectionally or bidirectionally from the origin to a defined sequence located at an appropriate part (appropriate for the specific replicon) of the genome or plasmid called a termination sequence (Ter site) where the replication complex is halted and replication terminated.

In order to correctly terminate replication at a Ter site, an organism must express a functional replication terminator protein (RTP). RTPs are nucleic acid binding proteins which bind to the Ter sites and form an RTP-Ter complex. The bound RTPs are believed to function in replication termination by preventing the helicase activity of the replication complex from unwinding the Ter site. This activity is termed a contrahelicase activity. RTPs and Ter sites have been identified in a wide variety of Gram positive and Gram negative microorganisms including, for example, Bacillus subtilis and Escherichia coli. (See Bussiere, et al., Mol. Micro. 31(6):1611-1618 (1999), Hill, J Biol Chem 272:26448-56 (1997), and Griffiths, et al., J. Bacteriology 180(13):3360-3367 (1998)).

The ability of most RTP-Ter complexes to halt replication is unidirectional; a replication complex approaching from one direction—the non-permissive direction—would be halted while one approaching from the opposite direction—the permissive direction—would be allowed to pass. With some modified RTPs the ability to halt replication is bi-directional and these RTPs can halt replication from either direction. Under normal—unidirectional—conditions, to achieve correct termination of replication, there are generally at least two Ter sites located on each genome or plasmid. The Ter sites are arranged so as to permit passage of a replication fork into the region between the Ter sites from either direction but prevent exit of the replication fork from the region. A replication complex will pass through a first Ter site and be stopped at a second Ter site while a replication complex approaching from the opposite direction will pass through the second site and be stopped at the first. This is shown schematically in FIG. 1.

RTPs have been found to bind Ter sites extremely tightly, resulting in very stable RTP-Ter complexes with long half lives. The high affinity of RTPs for Ter sites and the directionality of the Ter sites can be exploited for use in the methods and kits described in the present invention.

### SUMMARY OF THE INVENTION

The present invention provides materials and methods especially useful in molecular biology applications. Generally, the invention relates to use of one or more nucleic acid molecules comprising all or a portion of one or more Ter sites of the invention and/or one or more polypeptides comprising all or a portion of one or more Ter-binding proteins of the invention (e.g., RTPs) in vitro (e.g., outside a cell), in vivo (e.g., within a cell), or combinations thereof.

In one embodiment, the present invention relates to one or more nucleic acid molecules (which may be isolated) comprising all or a portion of at least one Ter site of the invention. Such nucleic acid molecules may be any form or type of nucleic acid molecule such as linear, circular, supercoiled, single stranded, double stranded, double stranded with one or more single stranded regions (e.g., at least one single stranded overhang at one or more termini of the molecules), etc. and may be isolated, part of a mixture and/or contained by one or more hosts or host cells. Such nucleic acid molecules may also comprise one or more components or sites selected from a group consisting of one or more recombination sites or portions thereof, one or more topoisomerase sites or portions thereof, one or more restriction enzyme recognition sites, one or more selectable markers, one or more origins of replication, one or more promoters, one or more open reading frames or partial open reading frames, one or more primer hybridization sites, one or more enhancers, one or more repressors, one or more transcription signals, one or more translation signals, and one or more tag sequences (e.g., six histidine tag, HA tag, GST tag, etc.). Preferred nucleic acid molecules of the invention include vectors, integration sequences (e.g., transposons), plasmids, cosmids, artificial chromosomes (e.g., BACs and YACs), phagemids and the like. Such Ter sites and/or portions thereof may be located at any position and in any orientation in the nucleic acid molecules of the invention including one or more positions within the molecules and/or at or near one or more termini of such molecules. In some embodiments, the nucleic acid molecules of the invention may optionally comprise one or more detectable atoms or groups or labels, for example, one or more radioisotopes, chromophores, fluorophores, enzymes, epitopes, haptens, antigens and/or combinations thereof. Such detectable molecules may be directly, indirectly, covalently and/or non-covalently bound to the nucleic acid molecules of the invention. In one aspect, the nucleic acid molecules of the invention may be bound to one or more Ter-binding proteins of the invention. The present invention also contemplates compositions comprising such nucleic acid molecules, reaction mixtures comprising such nucleic acid molecules, and host cells transformed with such nucleic acid molecules.

In one aspect, the present invention also contemplates proteins and/or polypeptides that bind to or interact with the Ter sites of the invention. Ter-binding proteins of the invention include, but are not limited to, wild-type Ter-binding proteins, mutants of wild-type Ter-binding proteins (e.g., point mutants, truncation mutants, insertion mutants, and combinations thereof), fragments of Ter-binding proteins that retain the ability to bind with a Ter-site of the invention, and combinations thereof (e.g., fragments of mutants). Ter-binding proteins of the present invention also comprise fusion proteins having one or more Ter-binding portions (i.e., wild-type, mutant, and/or fragment as described above) and one or more additional polypeptide portions. Ter-binding proteins of the invention also included modified Ter-binding proteins, for example, a Ter-binding protein (e.g., wild-type, mutant, fusion and/or fragment) comprising one or more modifying groups (e.g., labels, haptens, detectable moieties, and the like). Modifying groups may be directly, indirectly, covalently and/or non-covalently attached or bound to the Ter-binding proteins of the invention. Ter-binding proteins of the invention may comprise combinations of the above-described characteristics. For example, a Ter-binding protein of the invention may include one or more Ter-binding portions (e.g., wild-type, mutant, and/or fragments thereof), one or more additional polypeptide portions (i.e., fusions) and/or one or more modifying groups (e.g., detectable moieties, labels, etc.). Such one or more Ter-binding portions, one or more polypeptide portions, and/or one or more modifying groups may be arranged in any order and positioned in any location depending on need. For example, the modifying group(s) may be located on the Ter-binding portion(s), the additional polypeptide portion(s) or both. In addition, the additional polypeptide portion(s) may be located at the N-terminus and/or C-terminus of the Ter-binding portion(s) and/or may be located in the interior of the Ter-binding portion(s). The present invention also contemplates compositions comprising such Ter-binding proteins, reaction mixtures comprising such proteins, nucleic acids encoding such proteins and host cells transformed with such nucleic acid molecules.

In one aspect, the present invention provides a nucleic acid molecule comprising all or a portion of the one or more Ter sites of the invention flanked by recombination sites or portions thereof. In some embodiments, the recombination sites or portions thereof may be selected from a group consisting of att sites, lox sites, and/or FRT sites. The Ter sites of the invention may be selected from a group consisting of the Ter site sequences in Table 4. The present invention also relates to host cells comprising such nucleic acids. A host cell may express one or more Ter-binding proteins and/or one or more recombination proteins.

In some embodiments, the present invention provides methods for preparing nucleic acid molecules comprising all or a portion of one or more Ter sites of the invention. Thus, the invention relates to a method of synthesizing a nucleic acid molecule comprising:

(a) mixing one or more nucleic acid templates with one or more polypeptides having polymerase activity (e.g., DNA polymerase activity, reverse transcriptase activity, etc.) and one or more primers comprising all or a portion of one or more Ter sites of the invention; and

(b) incubating said mixture under conditions sufficient to synthesize one or more nucleic acid molecules which are complementary to all or a portion of said templates and which comprise all or a portion of one or more Ter sites of the invention. In accordance with the invention, the synthesized nucleic acid molecule comprising all or a portion of one or more Ter sites of the invention may be used as a template under appropriate conditions to synthesize nucleic acid molecules complementary to all or a portion of the Ter site containing templates, thereby forming double stranded molecules comprising all or a portion of one or more Ter sites of the invention. In one aspect, some or all of the synthesized nucleic acid molecules will comprise all or a portion of one or more Ter sites of the invention, preferably at or near one or both termini of the nucleic acid molecule. Preferably, such second synthesis step is performed in the presence of one or more primers comprising all or a portion of one or more Ter sites of the invention. In yet another aspect, the synthesized double stranded molecules may be amplified using primers which may comprise all or a portion of one or more Ter sites of the invention. In some embodiments, conditions sufficient to synthesize one or more nucleic acid molecules according to the invention may include one or more nucleotides, one or more buffers or buffering salts, one or more primers (which may comprise all or a portion of one or more Ter sites of the invention), one or more cofactors, and/or one or more additional polypeptides having a nucleotide polymerase activity. In some embodiments, methods of the invention may further comprise isolating one or more nucleic acid molecules produced by the methods of the invention, for example, by binding a nucleic acid molecule produced according to the invention with one or more molecules comprising all or a portion of one or more Ter-binding proteins of the invention and separating bound nucleic acids from unbound nucleic acids.

In some embodiments, the present invention provides a method of making cDNA molecules comprising all or a portion of one or more Ter sites of the invention. In accordance with the invention, cDNA molecules (single-stranded or double-stranded) may be prepared from a variety of nucleic acid template molecules. Preferred nucleic acid molecules for use in the present invention include single-stranded RNA molecules, as well as double-stranded DNA:RNA hybrids. More preferred nucleic acid molecules include messenger RNA (mRNA), transfer RNA (tRNA) and ribosomal RNA (rRNA) molecules, although mRNA molecules are the preferred template according to the invention. Such methods may comprise:

(a) mixing one or more RNA templates (e.g., mRNA) or a population of RNA templates with a polypeptide having polymerase activity and one or more primers comprising all or a portion of one or more Ter sites of the invention; and

(b) incubating said mixture under conditions sufficient to synthesize one or more nucleic acid molecules which are complementary to all or a portion of said templates and which comprise all or a portion of one or more Ter sites of the invention. In accordance with the invention, the synthesized nucleic acid molecule comprising one or more Ter sites of the invention may be used as a template under appropriate conditions to synthesize nucleic acid molecules complementary to all or a portion of the Ter site containing templates, thereby forming double stranded molecules comprising all or a portion of one or more Ter sites of the invention. In one aspect, some or all of the synthesized nucleic acid molecules will comprise all or a portion of one or more Ter sites of the invention, preferably at or near one or both termini of the nucleic acid molecule. Preferably, such second synthesis step is performed in the presence of one or more primers comprising all or a portion of one or more Ter sites of the invention. In yet another aspect, the synthesized double stranded molecules may be amplified using primers which may comprise all or a portion of one or more Ter sites of the invention. In some embodiments, conditions sufficient to produce a cDNA molecule according to the invention may include one or more nucleotides, one or more buffers or buffering salts, one or more primers (which may comprise all or a portion of one or more Ter sites of the invention), one or more cofactors, and/or one or more additional polypeptides having a nucleotide polymerase activity. In some embodiments, methods of the invention may further comprise isolating one or more cDNA molecules produced by the methods of the invention, for example, by binding a cDNA produced according to the invention with one or more molecules comprising all or a portion of one or more Ter-binding proteins of the invention and separating bound nucleic acids from unbound nucleic acids.

In another aspect of the invention, all or a portion of one or more Ter sites of the invention may be added to nucleic acid molecules by any of a number of nucleic acid amplification techniques. Such methods may comprise:

(a) mixing one or more templates with one or more primers comprising one or more Ter site of the invention and one or more polypeptides having polymerase activity; and

(b) incubating said mixture under conditions sufficient to amplify said one or more templates. In one aspect, some or all of the amplified templates will comprise one or more Ter site of the invention, preferably at or near one or both termini of the nucleic acid molecule.

In particular, such amplification methods may comprise:

(a) contacting a first nucleic acid molecule with a first primer molecule which is complementary to a portion of said first nucleic acid molecule and a second nucleic acid molecule with a second primer molecule which is complementary to a portion of said second nucleic acid molecule in the presence of one or more polypeptides having polymerases activity;

(b) incubating said molecules under conditions sufficient to form a third nucleic acid molecule complementary to all or a portion of said first nucleic acid molecule and a fourth nucleic acid molecule complementary to all or a portion of said second nucleic acid molecule;

(c) denaturing said first and third and said second and fourth nucleic acid molecules; and

(d) repeating steps (a) through (c) one or more times,

wherein said first and/or said second primer molecules comprise all or a portion one or more Ter sites of the invention. In some embodiments, such conditions according to the invention may include one or more nucleotides, one or more buffers or buffering salts, one or more primers (which may comprise all or a portion of one or more Ter sites of the invention), one or more cofactors, and/or one or more additional polypeptides having a nucleotide polymerase activity. In some embodiments, methods of the invention may further comprise isolating one or more nucleic acid molecules produced by the methods of the invention, for example, by binding a nucleic acid molecule produced according to the invention with one or more molecules comprising all or a portion of one or more Ter-binding proteins of the invention and separating bound nucleic acids from unbound nucleic acids.

In yet another aspect of the invention, a method for adding all or a portion of one or more Ter sites of the invention to nucleic acid molecules may comprise:

(a) contacting one or more nucleic acid molecules with one or more adapters or nucleic acid molecules which comprise all or a portion of one or more Ter sites of the invention; and

(b) incubating said mixture under conditions sufficient to add all or a portion of one or more Ter sites of the invention to said nucleic acid molecules. Preferably, linear molecules are used for adding such adapters or molecules in accordance with the invention and such adapters or molecules are preferably added to one or more termini of such linear molecules. The linear molecules may be prepared by any technique including mechanical (e.g., sonication or shearing) or enzymatic (e.g., polymerases, nucleases such as restriction endonucleases). Thus, the method of the invention may further comprise digesting the nucleic acid molecule with one or more nucleases (preferably any restriction endonucleases) and attaching (e.g., ligating, reacting with a topoisomerases and/or recombination proteins, etc.) one or more of the Ter site containing adapters or molecules to the molecule of interest. Molecules of interest and Ter site containing molecules may be blunt-ended or may have an overhanging end (i.e., sticky-ended) and the two molecules may be ligated together. Alternatively, topoisomerases and/or recombination proteins may be used to introduce Ter sites of the invention in accordance with the invention. Topoisomerases and/or recombination proteins cleave and rejoin nucleic acid molecules and therefore may be used in place of and/or in addition to nucleases and ligases. In some embodiments, such methods may further comprise isolating said nucleic acids comprising a Ter site, for example, by binding a nucleic acid molecule produced according to the invention with one or more molecules comprising all or a portion of one or more Ter-binding proteins of the invention and separating bound nucleic acids from unbound nucleic acids.

In another aspect, all or a portion of one or more Ter sites of the invention may be added to nucleic acid molecules by de novo synthesis. Thus, the invention relates to such a method which comprises chemically synthesizing one or more nucleic acid molecules in which all or a portion of one or more Ter sites of the invention are added by adding the appropriate sequence of nucleotides during the synthesis process. In some embodiments, such methods may further comprise isolating said nucleic acids comprising a Ter siteinv, for example, by binding a nucleic acid molecule produced according to the invention with one or more molecules comprising all or a portion of one or more Ter-binding proteins of the invention and separating bound nucleic acids from unbound nucleic acids.

In another embodiment of the invention, all or a portion of one or more Ter sites of the invention may be added to nucleic acid molecules of interest by a method which comprises:

(a) contacting one or more nucleic acid molecules with one or more integration sequences which comprise all or a portion of one or more Ter sites of the invention; and

(b) incubating said mixture under conditions sufficient to incorporate said Ter site containing integration sequences into said nucleic acid molecules. In accordance with this aspect of the invention, integration sequences may comprise any nucleic acid molecules which, through recombination or by integration, become a part of the nucleic acid molecule of interest. Integration sequences may be introduced in accordance with this aspect of the invention by in vivo or in vitro recombination (homologous recombination or illegitimate recombination) or by in vivo or in vitro installation by using transposons, insertion sequences, integrating viruses, homing introns, or other integrating elements. In some embodiments, such methods may further comprise isolating said nucleic acids comprising a Ter site of the invention, for example, by binding a nucleic acid molecule produced according to the invention with one or more molecules comprising all or a portion of one or more Ter-binding proteins of the invention and separating bound nucleic acids from unbound nucleic acids.

The present invention also includes compositions or reaction mixtures comprising one or more of the nucleic acid molecules of the invention. Such compositions or reaction mixtures may also comprise one or more other components for carrying out the methods of the invention. Such other components may include one or more Ter-binding proteins of the invention which may be bound and/or unbound to such one or more Ter sites of the invention or portions thereof, one or more ligases, one or more polymerases, one or more topoisomerases, one or more recombination proteins, one or more host cells (which may be competent to take up nucleic acid molecules), one or more supports (which may have one or more Ter-binding proteins and/or nucleic acid molecules comprising one or more Ter sites or portions thereof bound (e.g., directly or indirectly, covalently or non-covalently) to such support), and the like.

The present invention also includes compositions or reaction mixtures comprising all or a portion of one or more of the Ter-binding proteins of the invention. Such compositions or reaction mixtures may also comprise one or more other components for carrying out the methods of the invention. Such other components may include nucleic acids comprising all or a portion of one or more Ter sites of the invention which may be bound and/or unbound to such one or more Ter-binding proteins of the invention or portions thereof, one or more ligases, one or more polymerases, one or more topoisomerases, one or more recombination proteins, one or more host cells (which may be competent to take up nucleic acid molecules), one or more supports (which may have one or more Ter-binding proteins and/or nucleic acid molecules comprising one or more Ter sites or portions thereof bound (e.g., directly or indirectly, covalently or non-covalently) to such support), and the like.

In another aspect, the present invention relates to a modified protein comprising a Ter-binding protein of the invention and one or more modifications. In some aspects, the modifying group may be chemically attached to the Ter-binding protein of the invention. Ter-binding proteins of the invention may be wild-type Ter-binding proteins, mutants of wild-type Ter-binding proteins (e.g., point mutants, truncation mutants, insertion mutants, and combinations thereof), fragments of Ter-binding proteins that retain the ability to bind with a Ter-site of the invention, and combinations thereof (e.g., fragments of mutants). Ter-binding proteins of the present invention may also comprise fusion proteins having one or more Ter-binding portions (i.e., wild-type, mutant, and/or fragment as described above) and one or more additional polypeptide portions. The additional polypeptide portions maybe one or more enzymes, ligases, topoisomerase, recombination proteins, recombinases, polymerase (e.g., DNA polymerases, RNA polymerases, reverse transcriptases), tag sequences (e.g., 6-histidines, GST, HA, etc.), restriction enzymes, nucleases, binding polypeptides (e.g., antibodies and fragments thereof, such as Fabs, Fc, single stranded antibodies and fragments thereof), epitopes, antigens, haptens and the like and combinations, fragments, and mutants thereof. Fusion proteins may optionally comprise a linker between two portions, for example, between a Ter-binding portion and an enzyme portion. A linker may optionally comprise one or more cleavage sites, for example, a cleavage site for one or more proteolytic enzymes and/or one or more sites susceptible to chemical cleavage. Modifying groups may be any molecules known to those in the art (e.g., fluorophores, chromophores, haptens, ligands, etc.).

In another aspect, the present invention provides supports, which may be solid supports, to which are attached, directly or indirectly, covalently or non-covalently, nucleic acids and/or proteins of the present invention. In some embodiments, the supports of the present invention may comprise at least one oligonucleotide comprising all or a portion of one or more Ter sites of the invention. In some embodiments, the oligonucleotide may be in the form of a hairpin or stem-loop. In some embodiments, the supports of the present invention may comprise all or a portion or one or more Ter-binding proteins of the invention. In another aspect, the present invention includes compositions comprising supports of the present invention.

In a specific embodiment, the present invention relates to the use of at least one Ter sequence of the invention in one or more nucleic acid molecules for use with in vitro and/or in vivo cloning (preferably directional cloning). Thus, an aspect the invention allows for positive selection for nucleic acid molecules of interest (preferably those that have been cloned in a desired orientation). Cloning may be accomplished using any technique known in the art (e.g., restriction digest/ligation, recombinational cloning, topoisomerase-mediated cloning, TA cloning, and the like).

In one aspect, the present invention provides a method of cloning by providing at least one nucleic acid molecule of the invention comprising all or a portion of a Ter site of the invention and at least one vector, inserting or cloning all or a portion of said at least one nucleic acid molecule into said at least one vector, and selecting at least one vector comprising all or a portion of said at least one nucleic acid molecule in the desired orientation.

In another aspect the present invention provides a method of cloning by providing at least one vector comprising all or a portion of at least one Ter site of the invention and at least one nucleic acid molecule, inserting or cloning all or a portion of the at least one nucleic acid molecule into the at least one vector, and selecting at least one vector comprising all or a portion of the at least one nucleic acid molecule, preferably in the desired orientation (FIG. 2).

In another aspect, the present invention provides a method of cloning by providing at least one nucleic acid molecule of interest comprising all or a portion of at least one Ter site of the invention, providing at least one vector comprising all or a portion of at least one Ter site of the invention, inserting or cloning all or a portion of the at least one nucleic acid molecule into the at least one vector, and selecting at least one vector comprising all or a portion of the at least one nucleic acid molecule in the desired orientation (FIG. 3).

In some embodiments, the methods of the present invention may also comprise selecting against undesired nucleic acid molecules (including vectors). Such selections may involve selecting against molecules having all or a portion of a Ter site of the invention in a selectable conformation or orientation and/or selecting for molecules having all or a portion of a Ter site of the invention in a selectable conformation or orientation. In some embodiments, the selecting step comprises introducing (e.g., by transformation or transfection) the vector molecule into a host cell, wherein the host cell expresses at least one Ter-binding protein of the invention.

Thus, in one aspect, the present invention provides a method of directional insertion or cloning of nucleic acid molecules using one or more Ter sequences of the invention or portions thereof. In some embodiments, the desired orientation of the nucleic acid molecule in the vector is the orientation in which the Ter site of the invention in the nucleic acid molecule permits replication in the same direction as the Ter site of the invention in the vector. In this embodiment, at least one Ter site of the invention prevents replication of the vector when the nucleic acid molecule is in the undesired orientation (FIG. 3). In another embodiment, the desired orientation of the nucleic acid molecule in the vector avoids generation of a functional Ter site of the invention. In the undesired orientation, at least one functional Ter site is generated which prevents replication of the vector. Thus, for example, when the Ter site of the invention in the nucleic acid molecule and the Ter site of the invention in the vector are partial Ter sites, insertion of the nucleic acid molecule may or may not generate a functional Ter site of the invention, depending, e.g., on the orientation. In this case, the desired orientation will not generate a functional Ter site of the invention thus allowing replication of the recombinant vector.

The present invention also relates to the use of at least one Ter sequence of the invention or portions thereof to select against undesired nucleic acid molecules (FIG. 4). Like the positive selection methods of the invention, such method may be accomplished using in vitro and/or in vivo cloning of desired nucleic acid molecules. In one aspect the invention allows selection against undesired starting molecules and/or product molecules during in vitro or in vivo cloning. For example, the invention provides selection against a starting vector molecule which did not receive a desired insert. In another aspect, the invention provides for selection against intermediates which may be generated during cloning or insertion of nucleic acid molecules. Additionally, the invention provides for selection against undesired product molecules generated during cloning reactions.

In another aspect, the present invention relates to assuring a desired orientation of a nucleic acid insert (e.g., integration sequence, transposon, etc.) into a nucleic acid into which the insert is introduced. By controlling orientation, the whole nucleic acid construct will be allowed to replicate or prevented from replicating. For example, one or more inserts, e.g., transposons, can be contacted with a nucleic acid, e.g., plasmids, BACs, YACs, chromosomes, etc. If one or more of the inserts is in the desired orientation, replication will proceed through the sites that are in the permissive orientation. However, if an insert is oriented such that one or more Ter sites of the invention are in a non-permissive orientation, then replication will not be accomplished. Such methods are useful whenever an insertion orientation, e.g., the orientation of one or more transposons, is desired and may be especially effective in generating knockout vectors.

In another aspect, the present invention relates to methods for attaching (directly or indirectly, covalently or non-covalently) one or more nucleic acid molecules or populations of nucleic acid molecules to one or more supports (FIG. 5). Such methods may comprise binding (directly or indirectly, covalently or non-covalently) one or more Ter-binding proteins of the invention to one or more supports, and contacting the Ter-binding proteins of the invention with one or more nucleic acid molecules comprising one or more Ter sites of the invention, wherein the one or more Ter-binding proteins of the invention binds to the one or more nucleic acid molecules through interaction at the one or more Ter sites of the invention (or portions thereof). Bound nucleic acid molecules may then be used for further manipulation, for example, by interaction (e.g., hybridization) with one or more oligonucleotides (e.g., primers or probes) or interaction with peptides or proteins. Such manipulations may be more versatile and/or efficient compared to manipulations where other binding methods are used since the invention allows for binding of the nucleic acid molecule of interest to the support at one or more specific sites (depending on the location(s) of the Ter sites of the invention or portions thereof). Thus, a nucleic acid of interest may be attached in any orientation with respect to the support, i.e., 5′, 3′, and/or internal portion proximal to the support. Nucleic acids of the invention may have a double stranded region, a single stranded region and/or a part double stranded part single stranded region on either or both sides of the bound portion of the nucleic acid. In addition, nucleic acids of the present invention may be attached to a support at more than one position of the nucleic acid. This may allow the nucleic acid to be fixed in defined—optionally rigid—conformations on a support. Non-specific binding methods of the prior art (e.g., nucleic acid molecules at a number of undefined sites such as with the use of poly-lysine coated supports) are unable to accomplish attachment to a support in a defined orientation or conformation. This aspect of the invention thus may be advantageously used for nucleic acid isolation, for preparing nucleic acid arrays, and for constructing nanodevices.

In another aspect, the present invention relates to methods for attaching one or more Ter-binding proteins of the invention or populations of such proteins to one or more supports. Such methods may comprise binding one or more nucleic acid molecules comprising one or more Ter sequences of the invention or portions thereof to one or more supports, and/or contacting the nucleic acids with one or more Ter-binding proteins of the invention. In one aspect, the methods may comprise binding one or more nucleic acid molecules comprising one or more Ter sites of the invention with a support comprising one or more Ter-binding proteins of the invention. In another aspect, the methods may comprise binding one or more molecules, polypeptides or compounds comprising one or more Ter-binding proteins of the invention to one or more supports comprising one or more nucleic acid molecules that comprise one or more Ter sites of the invention. In another aspect, the interaction or binding or the Ter-binding proteins of the invention generally allows identification, isolation and/or purification of the nucleic acid molecules of the invention. The one or more Ter-binding proteins of the invention may bind to or interact with said one or more nucleic acid molecules through interaction at one or more Ter sites of the invention or portions thereof. A Ter-binding portion of a fusion protein may be used to, e.g., concentrate, harvest, isolate, etc. a desired component of the fusion protein. For example, a Ter-binding portion of a Ter-binding protein of the invention may serve as an isolation tag (e.g., affinity tag) and may be used to isolate or purify a molecule (e.g., polypeptide) to which it is fused or bound. In one aspect, the Ter-binding portion may bind to a nucleic acid molecule comprising all or a portion of a Ter site of the invention, which may be bound to a support, or to an antibody specific to the Ter-binding portion, which may be bound to a support. This allows the fusion protein to be isolated from other components in a biological sample. Preferred fusion proteins of this type may comprise a cleavage site that allows removal of the tag. Bound Ter-binding proteins and/or fusion proteins may then be further processed. Further processing may comprise, for example, elution and/or cleavage at one or more cleavage sites. In some embodiments, such bound Ter-binding proteins and/or fusion proteins may be interacted with one or more nucleic acid molecules or with other peptides or proteins while still bound to the support. In other embodiments, such Ter-binding proteins of the invention may be eluted from the support prior to further interactions. This aspect of the invention thus may be advantageously used for the isolation or purification of Ter-binding proteins and/or fusion proteins from any sample such as biological samples.

In another aspect, the present invention relates to a method for improving the transfection efficiency of one or more nucleic acid molecules, comprising providing a Ter site of the invention in the nucleic acid and contacting the nucleic acid with a Ter-binding protein of the invention. In some embodiments, the Ter-binding protein of the invention may comprise one or more receptor binding ligands. In some aspects, the present invention provides altered Ter-binding proteins comprising one or more cellular targeting sequences. In some preferred embodiments, one or more of the cellular targeting sequences may be a nuclear localization sequence.

In another aspect, the present invention relates to methods for enhancing the stability of a linear nucleic acid molecule in vivo, comprising providing a linear nucleic acid molecule, the nucleic acid molecule comprising Ter sites of the invention or portions thereof at or near one or both of its termini, contacting the nucleic acid with a Ter-binding protein of the invention to form a stable nucleic acid-protein complex and transfecting the stable nucleic acid-protein complex into a host cell, wherein the complex is more stable and/or more easily transfected than the nucleic acid transfected alone. In some embodiments, the linear nucleic acid comprises a coding sequence.

In another aspect, the present invention relates to a method for isolating a nucleic acid, comprising providing a mixture comprising one or more nucleic acid molecules, all or a portion of the nucleic acid molecules comprising all or a portion of one or more Ter sites of the invention, contacting the mixture with at least one composition, the composition comprising one or more Ter-binding proteins of the invention, wherein the one or more Ter-binding protein(s) binds to or interacts with the one or more Ter site(s), separating the nucleic acid from the mixture and isolating or purifying the nucleic acid (FIGS. 6A and 6B and FIG. 7). In some embodiments, the Ter-binding protein of the invention may be attached to a support. In yet another embodiment, the present invention provides improved methods for purification of nucleic acids, especially nucleic acid libraries. Generally, nucleic acids comprising a Ter site of the invention can be separated from other nucleic acids by methods of the present invention. One such embodiment is depicted in FIG. 6A which shows a stock vector with a stuffer fragment. To prepare vector reagent for library production, the stuffer fragment should be efficiently removed. The present invention provides methods for isolating the prepared vector reagent from stuffer fragments. For example, a stock vector can be constructed to comprise a Ter site of the invention in the stuffer fragment. After digestion with restriction enzymes, two cuts with one or more restriction enzyme will result in cleavage of stuffer from prepared reagent. Cuts at only one site or no cuts will leave the stuffer fragment still attached to the vector. Ter-binding protein of the invention, optionally bound to a support, can be used to effect separation of the stuffer fragments, uncut vectors, and singly cut vectors still comprising stuffer fragment from prepared vector reagent. Ter-binding proteins of the invention can be bound to any support, before, coincident with, or after being reacted with a vector digest. In another embodiment, nucleic acids containing a Ter site of the invention, such as uncut plasmids or singly-cut plasmids as well as undesired plasmid materials not containing the desired sequence of interest may thus be removed as shown in FIG. 6B.

In another embodiment, the presence of a Ter site of the invention in a template nucleic acid may used as shown in FIG. 7 to remove a template nucleic acid after completion of an amplification reaction, for example, a PCR reaction. The amplified sequence of interest may be the same as that of the template or may be a derivative thereof, e.g., a gene mutated by site directed mutagenesis. In a related aspect, compositions comprising a Ter-binding protein of the invention fused to a support may comprise, for example, a slide, a chip, a film, a bead, chromatography media, or a filter.

In another aspect, the present invention relates to methods for detecting a biological molecule, comprising the steps of contacting a biological molecule with a reagent, the reagent comprising a nucleic acid portion preferably containing at least one Ter site of the invention and a portion which forms a specific complex with the biological molecule, contacting the complex with a Ter-binding protein of the invention, optionally comprising a detection molecule, wherein the Ter-binding protein binds to the nucleic acid portions of the reagent, and detecting the bound Ter-binding protein, wherein the presence of the Ter-binding protein correlates to the presence of the biological molecule (FIG. 8). In some embodiments, the detection molecule may be selected from a group consisting of radioisotopes, chromophores, fluorophores, enzymes, antigens, haptens, epitopes and combinations thereof.

In another aspect, a biological molecule can be labeled or fused with a Ter-binding protein of the invention. The biological molecule can be, for example, a polynucleotide, a polypeptide, a polysaccharide, a lipid, or a phospholipid. The biological molecule can then be detected using a polynucleotide comprising a Ter site of the invention which is bound by the Ter-binding protein. This method of detection can be used to amplify a signal for detecting a molecule of interest, for example in an ELISA assay or in a western blot assay.

In yet another aspect, the present invention relates to a method for producing a desired fragment. The method includes binding a Ter-binding protein of the invention to the Ter site of the invention on a double-stranded DNA, digesting one strand of DNA with an exonuclease, where the bound Ter-binding protein blocks one strand from digestion with the enzyme. Optionally, the remaining undigested single-stranded DNA may be purified. This can be used to produce a single stranded (ss) DNA fragment from a double-stranded (ds) DNA containing a Ter site of the invention (FIG. 9). Optionally, the ssDNA can be converted to dsDNA or used to produce RNA. RNA yield can be increased by improving initiation efficiency to greater than about 90%, about 95%, in fact approaching 100%.

In yet another aspect, the present invention relates to a method for juxtaposing two sites in one or more nucleic acid molecules. In one embodiment of this type, a nucleic acid molecule comprising two Ter sites of the invention may be contacted with a multivalent (e.g., bivalent, trivalent, tetravalent, etc) Ter-binding protein of the invention (FIG. 11). Each Ter site of the invention may be bound by the Ter-binding protein thereby juxtaposing the sites. Those skilled in the art will appreciate that multiple nucleic acid molecules, each comprising a Ter site of the invention, may be juxtaposed in this fashion by contacting the nucleic acid molecules with a Ter-binding protein having the desired valency. In another embodiment, the present invention provides a method of juxtaposing two sites in a nucleic acid molecule, comprising providing a nucleic acid comprising a Ter site of the invention in proximity to a promoter, contacting the nucleic acid with a Ter-binding protein of the invention that is in functional association with a polymerase, and conducting a polymerization reaction. As shown in FIG. 10, a nucleic acid molecule comprising one or more Ter sites of the invention or portions thereof in proximity to one or more promoters may be contacted with a Ter-binding protein of the invention to which is attached a functional polymerase enzyme. The one or more Ter sites may be located such that the polymerase enzyme may functionally engage the promoter and, in the presence of the appropriate cofactors, perform a polymerization reaction. The Ter-binding protein preferably remains bound to the Ter site during the polymerization reaction and the polymerase reaction thus results in pulling the Ter site into proximity with a selected site on the nucleic acid molecule.

In yet another aspect, the present invention relates to a method for maintaining the topology of a nucleic acid molecule comprising two or more Ter sites of the invention. In some aspects, the invention provides a method of maintaining the superhelicity of a nucleic acid molecule, comprising contacting a nucleic acid comprising two or more Ter sites of the invention with a multivalent Ter-binding protein. In some embodiments, the nucleic acid may be a supercoiled dsDNA containing, e.g., two Ter sites of the invention one at each end of a segment desired to remain supercoiled after linearization (FIG. 11). A multivalent Ter-binding protein, such as a bivalent Ter-binding protein, is added such that both Ter sites can be bound and result in isolating one topological domain from another such that one domain can rotate independently of the other. Once the DNA fragment is linearized, the domain bounded by Ter sites of the invention remains in its pre-cleavage topology—supercoiled—until one of the Ter-binding sites is released by the multivalent Ter-binding protein or until the domain is cleaved. This method is useful for applications where supercoiling is beneficial. In some embodiments, the present invention provides a method of supercoiling a linear fragment, comprising contacting a fragment comprising two or more Ter sites of the invention with a multivalent Ter-binding protein to form a complex, and contacting the complex with a topoisomerase under conditions in which the topoisomerase supercoils the fragment.

In still another aspect, the present invention relates to a method for retaining ds DNA duplex under denaturing condition. This can be done by introducing a Ter site of the invention recognized by a cyclic or thermostable Ter-binding protein of the invention into the duplex DNA. Such thermostable Ter-binding protein of the invention may be preferably isolated from a thermophilic organism or by cyclizing or otherwise stabilizing a mesophilic Ter-binding protein.

In a similar aspect, the present invention provides a method for maintaining a clonal or “sticky end” in a PCR product wherein the primer contains an “overhanging” Ter site of the invention (FIG. 12). Such a ds Ter site could be distal to the amplified region with respect to the gene specific portion of the primer. The Ter site of the invention is bound by a Ter-binding protein which is thermostable. Once the PCR reaction is completed and deproteinized, the double stranded DNA product retains a Ter site overhang.

In another aspect, the present invention provides a method for detecting or measuring the proximity of agents to each other. For example, the present invention may be used in combination with fluorescence resonance energy transfer (FRET) to measure distances between two molecules of interest. In this method, a Ter-binding protein of the invention can be complexed with a molecule which binds the agents to be measured, such as an IgG molecule for example. The complexed Ter-binding proteins can be bound to Ter sites of the invention on nucleic acid molecules of a desired length. The nucleic acid molecules containing the Ter sites of the invention are labeled on the non-Ter-binding end of the molecule. The label can be such that when the two nucleic acid molecules are in close proximity, a change in intensity of label is detected, for example, the label is amplified, or the label is quenched. When the agents are bound by the complexed Ter-binding proteins described above, the distance of the agents can be determined after detecting the signal produced by the label used by knowing the distance occupied by the nucleic acid molecules. This method can be used to detect clustering of receptors of the surface of a cell.

### BRIEF DESCRIPTION OF THE FIGURES

FIG. 1 is a schematic representation of the replication of a plasmid containing Ter sites.

FIG. 2 is a schematic representation of the method for using a Ter sequence of the invention as a selectable marker. RS=recognition site (e.g., restriction site, recombination site, etc.), rep ori=origin of replication, arrow indicates direction of replication.

FIG. 3 is a schematic representation of a method for positive selection of a recombinant plasmid using a Ter sequence of the invention. GOI=DNA or gene of interest, solid black diamond=5′ end of Ter fragment, solid black circle=3′ end of Ter fragment, rep ori=origin of replication; arrow indicates direction of replication.

FIG. 4 is a schematic representation of a method for positive selection for insertion of desired nucleic acid and recombinant plasmids using a Ter sequence of the invention. GOI=DNA or gene of interest, solid black diamond=5′ end of Ter fragment, solid black circle=3′ end of Ter fragment, rep ori=origin of replication; arrow indicates direction of replication.

FIG. 5 is a schematic representation of the method for attaching nucleic acid to a solid support using a Ter sequence of the invention.

FIGS. 6A and 6B are schematic representations of methods for purifying a nucleic acid molecule using the Ter sequence of the invention.

FIG. 6A shows an embodiment where a Ter site (black box) is present on a stuffer fragment (wavy line) on a plasmid and permits removal of unreacted and partially reacted plasmid using a Ter-binding protein of the invention (TBP) attached to a solid support permitting purification of correctly reacted plasmid. FIG. 6B shows an embodiment where a Ter site of the invention (black box) is present on a plasmid and permits removal of unreacted and partially reacted plasmid from a reaction mixture reaction using a Ter-binding protein of the invention (TBP) attached to a solid support permitting purification of a desired nucleic acid of interest from a reaction mixture. RE=restriction enzyme, TBP=Ter-binding protein.

FIG. 7 is a schematic representation for a method for removing template containing a Ter site of the invention (black box) from the product of a polymerase chain reaction using a Ter-binding protein of the invention. TBP=Ter-binding protein.

FIG. 8 is a schematic representation of a method for target detection using a Ter sequence of the invention. TBP=Ter-binding protein, X=detection molecule if present.

FIG. 9 is a schematic representation for a method for producing single-stranded nucleic acids using a Ter sequence of the invention. TBP=Ter-binding protein.

FIG. 10 is a schematic representation for a method for apposing two ends of the same nucleic acid using a Ter sequence of the invention. T7=T7 RNA polymerase, TBP=Ter-binding protein.

FIG. 11 is a schematic representation for a method for maintaining superhelicity of a region of a linear nucleic acid using a Ter sequence of the invention. TBP=Ter-binding protein.

FIG. 12 is a schematic representation for a method for generating overhang “sticky ends” using Ter sequence of the invention. A=single stranded exploitable sequence, ter′=bottom strand of duplex Ter sequence, anneal=segment capable of annealing to template, ter=top strand of duplex ter sequence which hybridizes to ter′.

FIGS. 13A and 13B demonstrate results of analysis of recombinant vectors using directional cloning with Ter site of the invention. In 13A, the lanes were loaded as follows: M, one kb marker, lanes 1, 3, 5, 7, 9 11, 13, and 15, no insert; lanes 2, 4, 6, 8, 10, 12, 14, 16-24, 1 μl vector/5 μl insert. In 13B, the lanes were loaded as follows: M one kb marker, lanes 1-24, 10 μl vector/5 μl insert. +=correctly oriented insert, *=backwards insert, −=no insert, 0=no DNA evident.

FIG. 14 is a schematic of the construct used in Example 5.

FIG. 15 is a schematic representation of a vector of the invention containing two selectable markers.

FIG. 16 is a schematic representation of three vectors of the present invention.

### DETAILED DESCRIPTION OF THE PREFERRED EMBODIMENTS

### Definitions

In the description that follows, a number of terms used in recombinant DNA technology are extensively utilized. In order to provide a clearer and consistent understanding of the specification and claims, including the scope to be given such terms, the following definitions are provided. When a type of molecule is mention, unless contraindicated by the context, the term is seen to include the type of molecule mentioned as well as fragments and derivatives thereof.

Adapter: As used herein, an “adapter” is an oligonucleotide or nucleic acid fragment or segment (preferably DNA) which comprises all or a portion of one or more Ter sites. In some embodiments of the present invention, one or more adapters may be attached to one or more nucleic acid molecules of interest. Such adapters may be added at any location within a circular or linear molecule, although the adapters are preferably added at or near one or both termini of a linear molecule. In accordance with the invention, adapters may be added to nucleic acid molecules of interest by standard recombinant techniques (e.g., restriction digest and ligation, topoisomerase-mediated attachment, TA cloning, recombination protein-mediated attachment etc.). For example, adapters may be added to a circular molecule by first digesting the molecule with an appropriate restriction enzyme, adding the adapter at the cleavage site and reforming the circular molecule which contains the adapter(s) at the site of cleavage. Alternatively, adapters may be ligated directly to one or more and preferably both termini of a linear molecule thereby resulting in linear molecule(s) having adapters at one or both termini. In one aspect of the invention, adapters may be added to a population of linear molecules, (e.g., a cDNA library or genomic DNA which has been cleaved or digested) to form a population of linear molecules containing adapters at one or both termini of all or substantial portion of said population.

Vector: A nucleic acid that provides a useful biological or biochemical property to a nucleic acid sequence of interest, for example, an insert, a coding region, etc. Examples include plasmids, phages, and other nucleic acid sequences that are able to replicate or be replicated in vitro or in a host cell, or to convey a desired nucleic acid segment to a desired location within a host cell. A vector may comprise various sequences, for example, one or more recognition sites (e.g., restriction enzyme sites, recombination sites, topoisomerase sites, etc.) at which the vector sequences can be manipulated in a determinable fashion without loss of an essential biological function of the vector, and into which a nucleic acid fragment can be inserted, for example, to bring about its replication and/or cloning. Vectors can further provide primer sites, e.g., for PCR, transcriptional and/or translational initiation and/or regulation sites, recombinational signals, replicons, selectable markers, and other sequences known to those skilled in the art.

Cloning vector. A plasmid, cosmid, viral, or phage DNA or other DNA molecule which is able to replicate autonomously in a host cell, into which DNA may be spliced without loss of an essential biological function of the vector, in order to bring about its replication and cloning. The cloning vector may further contain a marker suitable for use in the identification of cells transformed with the cloning vector. Markers may be, for example, antibiotic resistance genes, e.g., tetracycline resistance or ampicillin resistance.

Expression vector. A vector similar to a cloning vector but which is capable of enhancing the expression of a gene which has been cloned into it, after transformation into a host. The cloned gene is usually placed under the control of (i.e., operably linked to) certain control sequences such as promoter sequences.

Fragment. A fragment is a molecule that is a portion of a larger molecule. A fragment may be obtained by cleavage of a larger molecule and/or by synthesis of less than all of the larger molecule. In some embodiments, a fragment may be a fragment of a Ter-binding protein and/or a Ter site of the invention. Fragments of the present invention may contain at least a portion of a larger molecule of the invention. Fragments of a protein may be produced by, for example, proteolysis of a larger protein, synthesis (e.g., solid phase synthesis) of an oligopeptide and/or transcription and translation from a nucleic acid encoding less than an entire protein. Fragments of nucleic acids may be produced by, for example, nuclease (e.g., endonuclease, exonuclease) treatment of a larger nucleic acid molecule, synthesis (e.g., solid phase synthesis) of an oligonucleotide, and/or amplification of a portion of a larger nucleic acid molecule (e.g., PCR). A fragment may be a set of fragments, the set, when properly juxtaposed, forming a complex or a larger molecule. Preferably, the set exhibits one or more functions of the larger molecule.

Recombinant host. Any prokaryotic or eukaryotic organism that contains the desired cloned genes in an expression vector, cloning vector or any DNA molecule. The term “recombinant host” is also meant to include those host cells which have been genetically engineered to contain the desired gene on the host chromosome or genome.

Host. Any prokaryotic or eukaryotic organism that is the recipient of a replicable expression vector, cloning vector or any DNA molecule. The DNA molecule may contain, but is not limited to, a structural gene, a promoter and/or an origin of replication.

Promoter. A DNA sequence recognized by an RNA polymerase for specific transcriptional initiation. Suitable promoters for use in the present invention include eukaryotic and prokaryotic promoters. Such promoters may be constitutive or regulatable (i.e., inducible or derepressible) promoters. Examples of constitutive promoters include the int promoter of bacteriophage λ, and the bla promoter of the β-lactamase gene of pBR322. Examples of inducible prokaryotic promoters include the major right and left promoters of bacteriophage λ (P_{R} and P_{L}), trp, recA, lacZ, lad, tet, gal, trc, ara BAD (Guzman, et al., 1995,J. Bacteriol. 177(14):4121-4130) and tac promoters of E. coli. The B. subtilis promoters include α-amylase (Ulmanen et al., J. Bacteriol 162:176-182 (1985)) and Bacillus bacteriophage promoters (Gryczan, T., In: The Molecular Biology Of Bacilli, Academic Press, New York (1982)). Streptomyces promoters are described by Ward et al., Mol. Gen. Genet. 203:468478 (1986)). Prokaryotic promoters are also reviewed by Glick, J. Ind. Microbiol. 1:277-282 (1987); Cenatiempto, Y., Biochimie 68:505-516 (1986); and Gottesman, Ann. Rev. Genet. 18:415-442 (1984). Expression in a prokaryotic cell also requires the presence of a ribosomal binding site upstream of the gene-encoding sequence. Such ribosomal binding sites are disclosed, for example, by Gold et al., Ann. Rev. Microbiol. 35:365404 (1981).

Gene. A nucleic acid sequence that contains information necessary for making a biological molecule, such as a polypeptide, protein or RNA. It may include a promoter and/or a structural gene as well as other sequences involved in expression of the molecule.

Polypeptide. As used herein, the term “polypeptide” refers to a sequence of contiguous amino acids, of any length. The terms “peptide,” “oligopeptide” or “protein” may be used interchangeably herein with the term “polypeptide.”

Derivative. A derivative of a polynucleotide is a molecule having at least 7, 8, or 9 or more preferably at least 10, 11, 12, 13, 14, or 15, or still more preferably 17, 18, 19, 20, 21, 22, 23, 24, or 25 nucleotides in the same sequence as one or more of the polynucleotides of the invention from which it is derived. One or more of the individual nucleotides of the polynucleotide of the invention may be replaced by one or more insertions, deletions or substitutions to form a derivative. The replacement will preferably not interfere with at least one function of the polynucleotide of the invention. The replacement may be at any position of the polynucleotide, i.e., either end or at an interior location. The replacement may alter one or more characteristics of the polynucleotide, for example, dissociation constant of the polynucleotide from one or more proteins of the invention and/or degradation rate—increase or decrease—of the derivative polynucleotide as compared to the polynucleotide from which it is derived. Suitable nucleotides for replacement are known to those of skill in the art and include, but are not limited to, those disclosed below.

A derivative of a polypeptide is a molecule having at least 4, 5, or 6, preferably 7, 8, 9, 10, 11, 12, 13, 14, or 15, more preferably 25, 50, 75, 100, 125, 150, 175, 200, or 250 amino acids in the same sequence as one or more of the polypeptides of the present invention from which it is derived. One or more of the individual amino acids of the polypeptide of the invention may be replaced by one or more insertions, deletions or substitutions to form a derivative. The replacement will preferably not interfere with at least one function of the polypeptide of the invention. The replacement may be at any position of the polypeptide, i.e., either end or at an interior location. In some embodiments, all or substantially all of one or more motifs, regions or domains may be deleted. For example, one or more loops—such as the L1 loop of Tus—may be deleted. A derivative may incorporate one or more insertions or substitutions of one or more amino acids—both natural and synthetic amino acids.

A derivative may have the same or different characteristics as the molecule from which it is derived. For example, a derivative polynucleotide may retain the ability to be bound by a wildtype Ter-binding protein. The affinity with which the derivative polynucleotide is bound may be the same as, greater than or lesser than the affinity with which the polynucleotide from which it is derived is bound. A derivative may be a multimer of the molecules—polynucleotides and/or polypeptides—of the invention. For example, a derivative may be a dimer, trimer, tetramer etc. of the molecules of the invention. A multimer may be comprised of identical or different monomeric units which may be of the same or different type. For example, a multimer may comprise two different polypeptides, two of the same polypeptides, or a polypeptide and a polynucleotide.

Operably linked. Operably linked means that a protein or nucleic acid element is positioned so as to influence or be influenced by another protein or nucleic acid element. The elements may be on the same or on different molecules.

Expression. Expression is the process by which a sequence of interest produces a polypeptide, protein or RNA. It includes transcription of the sequence into an RNA—which may be a messenger RNA (mRNA)—and may include the translation of such mRNA into one or more polypeptides. Those skilled in the art will appreciate that not all RNA molecules are translated into protein, for example ribosomal RNA, and expression in these cases would not include translation.

Substantially Pure. As used herein “substantially pure” means that the desired biomolecule is essentially free from contaminating cellular contaminants that are associated with the desired biomolecule in nature or in a recombinant host in which the biomolecule is produced. Contaminating cellular components may include, but are not limited to, nucleic acids, proteins, lipids and carbohydrates that are not desired.

Primer. As used herein “primer” refers to a single-stranded oligonucleotide that is extended by covalent bonding of nucleotide monomers during amplification or polymerization of a nucleic acid molecule.

Template. The term “template” as used herein refers to a nucleic acid molecule—single stranded DNA or RNA, double stranded DNA or RNA, RNA:DNA hybrids, populations of mRNA, polyA RNA, etc.—that is to be manipulated, for example, amplified, synthesized or sequenced. In some embodiments, a template may be a population of molecules (e.g., a population of mRNA molecules). In the case of a double-stranded nucleic acid molecule, denaturation of its strands to form a first and a second strand may be performed before further manipulations are performed. A primer, complementary to a portion of a template may be hybridized under appropriate conditions and then a nucleic acid polymerase may then synthesize a nucleic acid molecule complementary to all or a portion of the template. The newly synthesized molecule, according to the invention, may be longer, equal or shorter in length than the original template. Mismatch incorporation during the synthesis or extension of the newly synthesized nucleic acid molecule may result in one or a number of mismatched base pairs. In addition, the primer used need not be an exact match of the template sequence to which it hybridizes. Mis-matched bases in a primer may be used to effect site directed mutation in a sequence. Thus, the synthesized nucleic acid molecule need not be exactly complementary to the template.

Incorporating. The term “incorporating” as used herein means becoming a part of a nucleic acid molecule or primer.

Amplification. As used herein “amplification” refers to any in vitro method for increasing the number of copies of a nucleotide sequence with the use of a nucleic acid polymerase, for example, a DNA polymerase, an RNA polymerase and/or a reverse transcriptase. Nucleic acid amplification results in the incorporation of nucleotides into a nucleic acid molecule or primer thereby forming a new nucleic acid molecule complementary to—or substantially complementary to—a nucleic acid template. The newly formed nucleic acid molecule and its template can be used as templates to synthesize additional nucleic acid molecules. As used herein, one amplification reaction may consist of many rounds of nucleic acid replication. DNA amplification reactions include, for example, polymerase chain reactions (PCR). One PCR reaction may consist of, e.g., 5 to 100 “cycles” of denaturation and synthesis of a DNA molecule.

Oligonucleotide. “Oligonucleotide” refers to a synthetic or natural molecule comprising a covalently linked sequence of nucleotides which are joined by a phosphodiester bond between the 3N position of the pentose of one nucleotide and the 5N position of the pentose of the adjacent nucleotide.

Nucleotide. As used herein “nucleotide” refers to a base-sugar-phosphate combination. Nucleotides are monomeric units of a nucleic acid sequence (DNA and RNA). The term nucleotide includes deoxyribonucleoside triphosphates such as dATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs) and their derivatives. Illustrative examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Thermostable. As used herein “thermostable” refers to a Ter-binding protein that is resistant to inactivation by heat. Ter-binding proteins bind a Ter site on a nucleic acid molecule. For mesophilic Ter-binding proteins, the binding can be reduced—transiently or permanently—by heat treatment. As used herein, a thermostable Ter-binding activity is more resistant to heat inactivation than a mesophilic Ter-binding protein. However, a thermostable Ter-binding protein does not mean to refer to a protein that is totally resistant to heat inactivation and thus heat treatment may reduce the Ter-binding activity to some extent.

Hybridization. The terms “hybridization” and “hybridizing” refers to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used.

Ligation. The covalent attachment between a first and a second nucleotide sequence.

Target polynucleotide sequence. All or a portion of a sequence of nucleotides to be identified, the identity of which is known to a sufficient extent so as to allow the preparation of a binding polynucleotide sequence that is complementary to and will hybridize with such target polynucleotide sequence. The target polynucleotide sequence usually will contain from about 12 to 1000 or more nucleotides, preferably 15 to 50 nucleotides. The target polynucleotide sequence may or may not be a portion of a larger molecule.

Termination sequence. A termination sequence, or Ter site, is a nucleic acid molecule comprising a sequence of nucleotides that can be recognized—i.e., bound—by one or more Ter-binding protein or peptides and/or replication termination proteins or peptides.

Site-Specific Recombinase: As used herein, the phrase “site-specific recombinase” refers to a type of recombinase that typically has at least the following four activities (or combinations thereof): (1) recognition of specific nucleic acid sequences; (2) cleavage of said sequence or sequences; (3) topoisomerase activity involved in strand exchange; and (4) ligase activity to reseal the cleaved strands of nucleic acid (see Sauer, B., Current Opinions in Biotechnology 5:521-527 (1994)). Conservative site-specific recombination is distinguished from homologous recombination and transposition by a high degree of sequence specificity for both partners. The strand exchange mechanism involves the cleavage and rejoining of specific nucleic acid sequences in the absence of DNA synthesis (Landy, A. (1989) Ann. Rev. Biochem. 58:913-949).

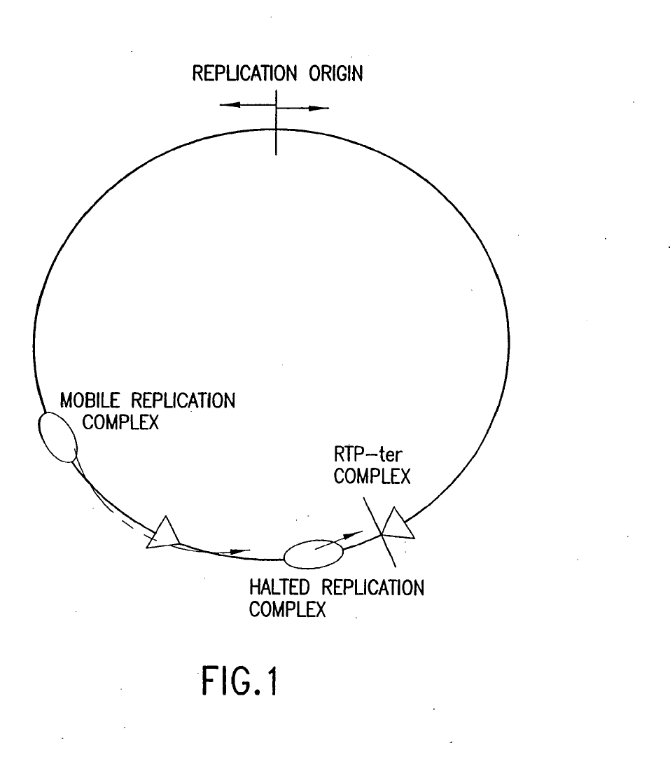

Recognition Sequence: As used herein, the phrase “recognition sequence” or “recognition site” refers to a particular sequence that is recognized (e.g., bound, cleaved, etc.) by a particular protein, chemical compound, DNA, or RNA molecule (e.g., restriction endonuclease, a modification methylase, topoisomerases, or a recombinase). In the present invention, a recognition sequence may refer to a recombination site, restriction enzyme site, and/or a topoisomerase site. For example, the recognition sequence for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other examples of recognition sequences are the attB, attP, attL, and attR sequences, which are recognized by the recombinase enzyme λ. Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis) (see Landy, Current Opinion in Biotechnology 3:699-707 (1993)). Such sites may also be engineered according to the present invention to enhance production of products in the methods of the invention. For example, when such engineered sites lack the P1 or H1 domains to make the recombination reactions irreversible (e.g., attR or attP), such sites may be designated attR′ or attP′ to show that the domains of these sites have been modified in some way.

Recombinational Cloning: As used herein, the phrase “recombinational cloning” refers to a method, such as that described in U.S. Pat. Nos. 5,888,732, 5,851,808, and 6,143,557 and in published PCT applications WO 01/05961 and WO 01/11058 (the contents of which are fully incorporated herein by reference), whereby segments of nucleic acid molecules or populations of such molecules are exchanged, inserted, replaced, substituted or modified, in vitro or in vivo. Preferably, such cloning method is an in vitro method.

Examples of cloning systems that utilize recombination at defined recombination sites have been previously described in U.S. Pat. No. 5,888,732, U.S. Pat. No. 6,143,557, U.S. Pat. No. 6,171,861, U.S. Pat. No. 6,270,969, and U.S. Pat. No. 6,277,608, and in pending U.S. application Ser. No. 09/517,466, and in published United States application no. 20020007051, all assigned to the Invitrogen Corporation, Carlsbad, Calif. A commercially available cloning system of this type is the GATEWAY™ Cloning System available from Invitrogen Corporation, Carlsbad, Calif. The GATEWAY™ Cloning System utilizes vectors that contain at least one recombination site to clone desired nucleic acid molecules in vivo or in vitro. In some embodiments, the system utilizes vectors that contain at least two different site-specific recombination sites that may be based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAY™ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

Recombination Proteins: As used herein, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include Cre, Int, IHF, Xis, Flp, Fis, Hin, Gin, (φ31, Cin, Tn3 resolvase, TndX, XerC, XerD, TnpX, Hjc, Gin, SpCCE1, and ParA.

Recombinases: As used herein, the term “recombinases” is used to refer to the protein that catalyzes strand cleavage and re-ligation in a recombination reaction. Site-specific recombinases are proteins that are present in many organisms (e.g., viruses and bacteria) and have been characterized as having both endonuclease and ligase properties. These recombinases (along with associated proteins in some cases) recognize specific sequences of bases in a nucleic acid molecule and exchange the nucleic acid segments flanking those sequences. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176) (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2μ circle plasmid (Broach, et al., Cell 29:227-234 (1982)).

Recombination site. A recombination site for use in the invention may be any nucleic acid that can serve as a substrate in a recombination reaction. Such recombination sites may be wild-type or naturally occurring recombination sites, or modified, variant, derivative, or mutant recombination sites. Examples of recombination sites for use in the invention include, but are not limited to, phage-lambda recombination sites (such as attP, attB, attL, and attR and mutants or derivatives thereof) and recombination sites from other bacteriophages such as phi80, P22, P2, 186, P4 and P1 (including lox sites such as loxP and loxP511).

Preferred recombination proteins and mutant, modified, variant, or derivative recombination sites for use in the invention include those described in U.S. Pat. Nos. 5,888,732, 5,851,808, 6,143,557, 6,171,861, 6,270,969, and 6,277,608 and in U.S. application Ser. No. 09/438,358 (filed Nov. 12, 1999), based upon U.S. provisional application No. 60/108,324 (filed Nov. 13, 1998). Mutated att sites (e.g., attB 1-10, attP 1-10, attR 1-10 and attL 1-10) are described in U.S. provisional patent application Nos. 60/122,389, filed Mar. 2, 1999, 60/126,049, filed Mar. 23, 1999, 60/136,744, filed May 28, 1999, 60/169,983, filed Dec. 10, 1999, and 60/188,000, filed Mar. 9, 2000, and in U.S. application Ser. No. 09/517,466, filed Mar. 2, 2000, and 09/732,914, filed Dec. 11, 2000 (published as 20020007051-A1) and in published PCT applications WO 01/05961 and WO 01/11058 the disclosures of which are specifically incorporated herein by reference in their entirety. Other suitable recombination sites and proteins are those associated with the GATEWAY™ Cloning Technology available from Invitrogen Corporation, Carlsbad, Calif., and described in the product literature of the GATEWAY™ Cloning Technology, the entire disclosures of all of which are specifically incorporated herein by reference in their entireties.

Sites that may be used in the present invention include att sites. The 15 bp core region of the wildtype att site (GCTTTTTTAT ACTAA (SEQ ID NO:)), which is identical in all wildtype att sites, may be mutated in one or more positions. Other att sites that specifically recombine with other att sites can be constructed by altering nucleotides in and near the 7 base pair overlap region, bases 6-12 of the core region. Thus, recombination sites suitable for use in the methods, molecules, compositions, and vectors of the invention include, but are not limited to, those with insertions, deletions or substitutions of one, two, three, four, or more nucleotide bases within the 15 base pair core region (see U.S. application Ser. No. 08/663,002, filed Jun. 7, 1996 (now U.S. Pat. No. 5,888,732) and 09/177,387, filed Oct. 23, 1998, which describes the core region in further detail, and the disclosures of which are incorporated herein by reference in their entireties). Recombination sites suitable for use in the methods, compositions, and vectors of the invention also include those with insertions, deletions or substitutions of one, two, three, four, or more nucleotide bases within the 15 base pair core region that are at least 50% identical, at least 55% identical, at least 60% identical, at least 65% identical, at least 70% identical, at least 75% identical, at least 80% identical, at least 85% identical, at least 90% identical, or at least 95% identical to this 15 base pair core region.

As a practical matter, whether any particular nucleic acid molecule is at least 50%, 60%, 70%, 75%, 80%, 85%, 90%, 95%, 96%, 97%, 98% or 99% identical to, for instance, a given recombination site nucleotide sequence or portion thereof can be determined conventionally using known computer programs such as DNAsis software (Hitachi Software, San Bruno, Calif.) for initial sequence alignment followed by ESEE version 3.0 DNA/protein sequence software (cabot@trog.mbb.sfu.ca) for multiple sequence alignments. Alternatively, such determinations may be accomplished using the BESTFIT program (Wisconsin Sequence Analysis Package, Genetics Computer Group, University Research Park, 575 Science Drive, Madison, Wis. 53711), which employs a local homology algorithm (Smith and Waterman, Advances in Applied Mathematics 2: 482-489 (1981)) to find the best segment of homology between two sequences. When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed. Computer programs such as those discussed above may also be used to determine percent identity and homology between two proteins at the amino acid level.

Analogously, the core regions in attB1, attP1, attL1 and attR1 are identical to one another, as are the core regions in attB2, attP2, attL2 and attR2. Nucleic acid molecules suitable for use with the invention also include those comprising insertions, deletions or substitutions of one, two, three, four, or more nucleotides within the seven base pair overlap region (TTTATAC, bases 6-12 in the core region). The overlap region is defined by the cut sites for the integrase protein and is the region where strand exchange takes place. Examples of such mutants, fragments, variants and derivatives include, but are not limited to, nucleic acid molecules in which (1) the thymine at position 1 of the seven by overlap region has been deleted or substituted with a guanine, cytosine, or adenine; (2) the thymine at position 2 of the seven by overlap region has been deleted or substituted with a guanine, cytosine, or adenine; (3) the thymine at position 3 of the seven by overlap region has been deleted or substituted with a guanine, cytosine, or adenine; (4) the adenine at position 4 of the seven by overlap region has been deleted or substituted with a guanine, cytosine, or thymine; (5) the thymine at position 5 of the seven by overlap region has been deleted or substituted with a guanine, cytosine, or adenine; (6) the adenine at position 6 of the seven by overlap region has been deleted or substituted with a guanine, cytosine, or thymine; and (7) the cytosine at position 7 of the seven by overlap region has been deleted or substituted with a guanine, thymine, or adenine; or any combination of one or more (e.g., two, three, four, five, etc.) such deletions and/or substitutions within this seven by overlap region. The nucleotide sequences of representative seven base pair core regions are set out below.

Altered att sites have been constructed that demonstrate that (1) substitutions made within the first three positions of the seven base pair overlap (TTTATAC) strongly affect the specificity of recombination, (2) substitutions made in the last four positions (TTTATAC) only partially alter recombination specificity, and (3) nucleotide substitutions outside of the seven by overlap, but elsewhere within the 15 base pair core region, do not affect specificity of recombination but do influence the efficiency of recombination. Thus, nucleic acid molecules and methods of the invention include those comprising or employing one, two, three, four, five, six, eight, ten, or more recombination sites which affect recombination specificity, particularly one or more (e.g., one, two, three, four, five, six, eight, ten, twenty, thirty, forty, fifty, etc.) different recombination sites that may correspond substantially to the seven base pair overlap within the 15 base pair core region, having one or more mutations that affect recombination specificity. Particularly preferred such molecules may comprise a consensus sequence such as NNNATAC wherein “N” refers to any nucleotide (i.e., may be A, G, T/U or C). Preferably, if one of the first three nucleotides in the consensus sequence is a T/U, then at least one of the other two of the first three nucleotides is not a T/U.

The core sequence of each att site (attB, attP, attL and attR) can be divided into functional units consisting of integrase binding sites, integrase cleavage sites and sequences that determine specificity. Specificity determinants are defined by the first three positions following the integrase top strand cleavage site. These three positions are shown with underlining in the following reference sequence: CAACTTTTTTATAC AAAGTTG (SEQ ID NO:27). Modification of these three positions (64 possible combinations) can be used to generate att sites that recombine with high specificity with other att sites having the same sequence for the first three nucleotides of the seven base pair overlap region. The possible combinations of first three nucleotides of the overlap region are shown in Table 1.

TABLE 1
Modifications of the First Three
Nucleotides of the att Site Seven
Base Pair Overlap Region that
Alter Recombination Specificity.

| | | | |
|---|---|---|---|
| AAA | CAA | GAA | TAA |
| AAC | CAC | GAC | TAC |
| AAG | CAG | GAG | TAG |
| AAT | CAT | GAT | TAT |
| ACA | CCA | GCA | TCA |
| ACC | CCC | GCC | TCC |
| ACG | CCG | GCG | TCG |
| ACT | CCT | GCT | TCT |
| AGA | CGA | GGA | TGA |
| AGC | CGC | GGC | TGC |
| AGG | CGG | GGG | TGG |
| AGT | CGT | GGT | TGT |
| ATA | CTA | GTA | TTA |
| ATC | CTC | GTC | TTC |
| ATG | CTG | GTG | TTG |
| ATT | CTT | GTT | TTT |

Representative examples of seven base pair att site overlap regions suitable for in methods, compositions and vectors of the invention are shown in Table 2. The invention further includes nucleic acid molecules comprising one or more (e.g., one, two, three, four, five, six, eight, ten, twenty, thirty, forty, fifty, etc.) nucleotides sequences set out in Table 2. Thus, for example, in one aspect, the invention provides nucleic acid molecules comprising the nucleotide sequence GAAATAC, GATATAC, ACAATAC, or TGCATAC.

TABLE 2
Representative Examples of Seven
Base Pair att Site Overlap
Regions Suitable for use in the
recombination sites of the Invention.

| | | | |
|---|---|---|---|
| AAAATAC | CAAATAC | GAAATAC | TAAATAC |
| AACATAC | CACATAC | GACATAC | TACATAC |
| AAGATAC | CAGATAC | GAGATAC | TAGATAC |
| AATATAC | CATATAC | GATATAC | TATATAC |
| ACAATAC | CCAATAC | GCAATAC | TCAATAC |
| ACCATAC | CCCATAC | GCCATAC | TCCATAC |
| ACGATAC | CCGATAC | GCGATAC | TCGATAC |
| ACTATAC | CCTATAC | GCTATAC | TCTATAC |
| AGAATAC | CGAATAC | GGAATAC | TGAATAC |
| AGCATAC | CGCATAC | GGCATAC | TGCATAC |
| AGGATAC | CGGATAC | GGGATAC | TGGATAC |
| AGTATAC | CGTATAC | GGTATAC | TGTATAC |
| ATAATAC | CTAATAC | GTAATAC | TTAATAC |
| ATCATAC | CTCATAC | GTCATAC | TTCATAC |
| ATGATAC | CTGATAC | GTGATAC | TTGATAC |
| ATTATAC | CTTATAC | GTTATAC | TTTATAC |

As noted above, alterations of nucleotides located 3′ to the three base pair region discussed above can also affect recombination specificity. For example, alterations within the last four positions of the seven base pair overlap can also affect recombination specificity.

For example, mutated att sites that may be used in the practice of the present invention include attB1 (AGCCTGCTTT TTTGTACAAA CTTGT (SEQ ID NO:28)), attP1 (TACAGGTCAC TAATACCATC TAAGTAGTTG ATTCATAGTG ACTGGATATG TTGTGTTTTA CAGTATTATG TAGTCTGTTT TTTATGCAAA ATCTAATTTA ATATATTGAT ATTTATATCA TTTTACGTTT CTCGTTCAGC TTTTTTGTAC AAAGTTGGCA TTATAAAAAA GCATTGCTCA TCAATTTGTT GCAACGAACA GGTCACTATC AGTCAAAATA AAATCATTAT TTG (SEQ ID NO:29)), attL1 (CAAATAATGA TTTTATTTTG ACTGATAGTG ACCTGTTCGT TGCAACAAAT TGATAAGCAA TGCTTTTTTA TAATGCCAAC TTTGTACAAA AAAGCAGGCT (SEQ ID NO:30)), and attR1 (ACAAGTTTGT ACAAAAAAGC TGAACGAGAAACGTAAAATG ATATAAATAT CAATATATTA AATTAGATTT TGCATAAAAA ACAGACTACA TAATACTGTA AAACACAACA TATCCAGTCA CTATG (SEQ ID NO:31)). Table 3 provides the sequences of the regions surrounding the core region for the wild type att sites (attB0, P0, R0, and L0) as well as a variety of other suitable recombination sites. Those skilled in the art will appreciated that the remainder of the site may be the same as the corresponding site (B, P, L, or R) listed above.

TABLE 3
Nucleotide sequences of att sites.

| | | |
|---|---|---|
| attB0 | AGCCTGCTTT TTTATACTA | (SEQ ID NO: 32) |
| | CTTGAGC | |
| attP0 | GTTCAGCTTT TTTATACTAA | (SEQ ID NO: 33) |
| | GTTGGCA | |
| attL0 | AGCCTGCTTT TTTATACTAA | (SEQ ID NO: 34) |
| | GTTGGCA | |
| attR0 | GTTCAGCTTT TTTATACTAA | (SEQ ID NO: 35) |
| | CTTGAGC | |
| attB1 | AGCCTGCTTT TTTGTACAAA | (SEQ ID NO: 36) |
| | CTTGT | |
| attP1 | GTTCAGCTTT TTTGTACAAA | (SEQ ID NO: 37) |
| | GTTGGCA | |
| attL1 | AGCCTGCTTT TTTGTACAAA | (SEQ ID NO: 38) |
| | GTTGGCA | |
| attR1 | GTTCAGCTTT TTTGTACAAA | (SEQ ID NO: 39) |
| | CTTGT | |
| attB2 | ACCCAGCTTT CTTGTACAAA | (SEQ ID NO: 40) |
| | GTGGT | |
| attP2 | GTTCAGCTTT CTTGTACAAA | (SEQ ID NO: 41) |
| | GTTGGCA | |
| attL2 | ACCCAGCTTT CTTGTACAAA | (SEQ ID NO: 42) |
| | GTTGGCA | |
| attR2 | GTTCAGCTTT CTTGTACAAA | (SEQ ID NO: 43) |
| | GTGGT | |
| attB5 | CAACTTTATT ATACAAAGTT | (SEQ ID NO: 44) |
| | GT | |
| attP5 | GTTCAACTTT ATTATACAAA | (SEQ ID NO: 45) |
| | GTTGGCA | |
| attL5 | CAACTTTATT ATACAAAGTT | (SEQ ID NO: 46) |
| | GGCA | |
| attR5 | GTTCAACTTT ATTATACAAA | (SEQ ID NO: 47) |
| | GTTGT | |
| attB11 | CAACTTTTCT ATACAAAGTT | (SEQ ID NO: 48) |
| | GT | |
| attP11 | GTTCAACTTT TCTATACAAA | (SEQ ID NO: 49) |
| | GTTGGCA | |
| attL11 | CAACTTTTCT ATACAAAGTT | (SEQ ID NO: 50) |
| | GGCA | |
| attR11 | GTTCAACTTT TCTATACAAA | (SEQ ID NO: 51) |
| | GTTGT | |
| attB17 | CAACTTTTGT ATACAAAGTT | (SEQ ID NO: 52) |
| | GT | |
| attP17 | GTTCAACTTT TGTATACAAA | (SEQ ID NO: 53) |
| | GTTGGCA | |
| attL17 | CAACTTTTGT ATACAAAGTT | (SEQ ID NO: 54) |
| | GGCA | |
| attR17 | GTTCAACTTT TGTATACAAA | (SEQ ID NO: 55) |
| | GTTGT | |
| attB19 | CAACTTTTTC GTACAAAGT | (SEQ ID NO: 56) |
| | GT | |
| attP19 | GTTCAACTTT TTCGTACAAA | (SEQ ID NO: 57) |
| | GTTGGCA | |
| attL19 | CAACTTTTTC GTACAAAGTT | (SEQ ID NO: 58) |
| | GGCA | |
| attR19 | GTTCAACTTT TTCGTACAAA | (SEQ ID NO: 59) |
| | GTTGT | |
| attB20 | CAACTTTTTG GTACAAAGTT | (SEQ ID NO: 60) |
| | GT | |
| attP20 | GTTCAACTTT TTGGTACAAA | (SEQ ID NO: 61) |
| | GTTGGCA | |
| attL20 | CAACTTTTTG GTACAAAGTT | (SEQ ID NO: 62) |
| | GGCA | |
| attR20 | GTTCAACTTT TTGGTACAAA | (SEQ ID NO: 63) |
| | GTTGT | |
| attB21 | CAACTTTTTA ATACAAAGTT | (SEQ ID NO: 64) |
| | GT | |
| attP21 | GTTCAACTTT TTAATACAAA | (SEQ ID NO: 65) |
| | GTTGGCA | |
| attL21 | CAACTTTTTA ATACAAAGTT | (SEQ ID NO: 66) |
| | GGCA | |
| attR21 | GTTCAACTTT TTAATACAAA | (SEQ ID NO: 67) |
| | GTTGT | |

Other recombination sites having unique specificity (i.e., a first site will recombine with its corresponding site and will not substantially recombine with a second site having a different specificity) are known to those skilled in the art and may be used to practice the present invention. Corresponding recombination proteins for these systems may be used in accordance with the invention with the indicated recombination sites. Other systems providing recombination sites and recombination proteins for use in the invention include the FLP/FRT system from Saccharomyces cerevisiae, the resolvase family (e.g., γδ, TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases and the psi, dif and cer recombination sites in E. coli. Other suitable recombination sites may be found in U.S. Pat. No. 5,851,808 issued to Elledge and Liu which is specifically incorporated herein by reference.

The materials and methods of the invention may further encompass the use of “single use” recombination sites which undergo recombination one time and then either undergo recombination with low frequency (e.g., have at least five fold, at least ten fold, at least fifty fold, at least one hundred fold, or at least one thousand fold lower recombination activity in subsequent recombination reactions) or are essentially incapable of undergoing recombination. The invention also provides methods for making and using nucleic acid molecules which contain such single use recombination sites and molecules which contain these sites. Examples of methods which can be used to generate and identify such single use recombination sites are set out in PCT/US00/21623, published as WO 01/11058, which claims priority to U.S. provisional patent application 60/147,892, filed Aug. 9, 1999, both of which are specifically incorporated herein by reference.

Topoisomerase recognition site. As used herein, the term “topoisomerase recognition site” or “topoisomerase site” means a defined nucleotide sequence that is recognized and bound by a site specific topoisomerase. For example, the nucleotide sequence 5′-(C/T)CCTT-3′ is a topoisomerase recognition site that is bound specifically by most poxvirus topoisomerases, including vaccinia virus DNA topoisomerase I, which then can cleave the strand after the 3′-most thymidine of the recognition site to produce a nucleotide sequence comprising 5′-(C/T)CCTT-PO₄-TOPO, i.e., a complex of the topoisomerase covalently bound to the 3′ phosphate through a tyrosine residue in the topoisomerase (see Shuman, J. Biol. Chem. 266:11372-11379, 1991; Sekiguchi and Shuman, Nucl. Acids Res. 22:5360-5365, 1994; U.S. Pat. No. 5,766,891; PCT/US95/16099; and PCT/US98/12372). In comparison, the nucleotide sequence 5′-GCAACTT-3′ is the topoisomerase recognition site for type IA E. coli topoisomerase III.

Topoisomerases are categorized as type I, including type IA and type topoisomerases, which cleave a single strand of a double stranded nucleic acid molecule, and type II topoisomerases (gyrases), which cleave both strands of a nucleic acid molecule. Type IA and IB topoisomerases cleave one strand of a nucleic acid molecule. Cleavage of a nucleic acid molecule by type IA topoisomerases generates a 5′ phosphate and a 3′ hydroxyl at the cleavage site, with the type IA topoisomerase covalently binding to the 5′ terminus of a cleaved strand. In comparison, cleavage of a nucleic acid molecule by type IB topoisomerases generates a 3′ phosphate and a 5′ hydroxyl at the cleavage site, with the type IB topoisomerase covalently binding to the 3′ terminus of a cleaved strand. As disclosed herein, type I and type II topoisomerases, as well as catalytic domains and mutant forms thereof, are useful for generating double stranded recombinant nucleic acid molecules covalently linked in both strands according to a method of the invention.

Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases (see Berger, Biochim. Biophys. Acta 1400:3-18, 1998; DiGate and Marians, J. Biol. Chem. 264:17924-17930, 1989; Kim and Wang, J. Biol. Chem. 267:17178-17185, 1992; Wilson, et al., J. Biol. Chem. 275:1533-1540, 2000; Hanai, et al., Proc. Natl. Acad. Sci., USA 93:3653-3657, 1996, U.S. Pat. No. 6,277,620, each of which is incorporated herein by reference). E. coli topoisomerase III, which is a type IA topoisomerase that recognizes, binds to and cleaves the sequence 5′-GCAACTT-3′, can be particularly useful in a method of the invention (Zhang, et al., J. Biol. Chem. 270:23700-23705, 1995, which is incorporated herein by reference). A homolog, the traE protein of plasmid RP4, has been described by Li, et al., J. Biol. Chem. 272:19582-19587 (1997) and can also be used in the practice of the invention. A DNA-protein adduct is formed with the enzyme covalently binding to the 5′-thymidine residue, with cleavage occurring between the two thymidine residues.

Type IB topoisomerases include the nuclear type I topoisomerases present in all eukaryotic cells and those encoded by vaccinia and other cellular poxviruses (see Cheng, et al., Cell 92:841-850, 1998, which is incorporated herein by reference). The eukaryotic type IB topoisomerases are exemplified by those expressed in yeast, Drosophila and mammalian cells, including human cells (see Caron and Wang, Adv. Pharmacol. 29B,:271-297, 1994; Gupta, et al., Biochim. Biophys. Acta 1262:1-14, 1995, each of which is incorporated herein by reference; see, also, Berger, supra, 1998). Viral type IB topoisomerases are exemplified by those produced by the vertebrate poxviruses (vaccinia, Shope fibroma virus, ORF virus, fowlpox virus, and molluscum contagiosum virus), and the insect poxvirus (Amsacta moorei entomopoxvirus) (see Shuman, Biochim. Biophys. Acta 1400:321-337, 1998; Petersen, et al., Virology 230:197-206, 1997; Shuman and Prescott, Proc. Natl. Acad. Sci., USA 84:7478-7482, 1987; Shuman, J. Biol. Chem. 269:32678-32684, 1994; U.S. Pat. No. 5,766,891; PCT/US95/16099; PCT/US98/12372, each of which is incorporated herein by reference; see, also, Cheng, et al., supra, 1998).

Type II topoisomerases include, for example, bacterial gyrase, bacterial DNA topoisomerase IV, eukaryotic DNA topoisomerase II, and T-even phage encoded DNA topoisomerases (Roca and Wang, Cell 71:833-840, 1992; Wang, J. Biol. Chem. 266:6659-6662, 1991, each of which is incorporated herein by reference; Berger, supra, 1998). Like the type IB topoisomerases, the type II topoisomerases have both cleaving and ligating activities. In addition, like type IB topoisomerase, substrate nucleic acid molecules can be prepared such that the type II topoisomerase can form a covalent linkage to one strand at a cleavage site. For example, calf thymus type II topoisomerase can cleave a substrate nucleic acid molecule containing a 5′ recessed topoisomerase recognition site positioned three nucleotides from the 5′ end, resulting in dissociation of the three nucleotide sequence 5′ to the cleavage site and covalent binding the of the topoisomerase to the 5′ terminus of the nucleic acid molecule (Andersen, et al., supra, 1991). Furthermore, upon contacting such a type II topoisomerase charged nucleic acid molecule with a second nucleotide sequence containing a 3′ hydroxyl group, the type II topoisomerase can ligate the sequences together, and then is released from the recombinant nucleic acid molecule. As such, type II topoisomerases also are useful for performing methods of the invention.

The various topoisomerases exhibit a range of sequence specificity. For example, type II topoisomerases can bind to a variety of sequences, but cleave at a highly specific recognition site (see Andersen, et al., J. Biol. Chem. 266:9203-9210, 1991, which is incorporated herein by reference.). In comparison, the type LB topoisomerases include site specific topoisomerases, which bind to and cleave a specific nucleotide sequence (“topoisomerase recognition site”). Upon cleavage of a nucleic acid molecule by a topoisomerase, for example, a type IB topoisomerase, the energy of the phosphodiester bond is conserved via the formation of a phosphotyrosyl linkage between a specific tyrosine residue in the topoisomerase and the 3′ nucleotide of the topoisomerase recognition site. Where the topoisomerase cleavage site is near the 3′ terminus of the nucleic acid molecule, the downstream sequence (3′ to the cleavage site) can dissociate, leaving a nucleic acid molecule having the topoisomerase covalently bound to the newly generated 3′ end.

In one aspect, the present invention provides methods for linking a first and at least a second nucleic acid segment (either or both of which may contain all or a portion of one or more Ter sites and/or sequences of interest) with at least one (e.g., 1, 2, 3, 4, 5, 6, 7, 8, 9, 10, etc.) topoisomerase (e.g., a type IA, type IB, and/or type II topoisomerase) such that either one or both strands of the linked segments are covalently joined at the site where the segments are linked.

A method for generating a double stranded recombinant nucleic acid molecule covalently linked in one strand can be performed by contacting a first nucleic acid molecule which has a site-specific topoisomerase recognition site (e.g., a type IA. IB, and/or a type II topoisomerase recognition site), or a cleavage product thereof, at a 5′ or 3′ terminus, with a second (or other) nucleic acid molecule, and optionally, a topoisomerase (e.g., a type IA, type IB, and/or type II topoisomerase), such that the second nucleotide sequence can be covalently attached to the first nucleotide sequence. As disclosed herein, the methods of the invention can be performed using any number of nucleotide sequences, typically nucleic acid molecules wherein at least one of the nucleotide sequences has a site-specific topoisomerase recognition site (e.g., a type IA, type IB or type II topoisomerase), or cleavage product thereof, at one or both 5′ and/or 3′ termini.

In some embodiments, two double-stranded nucleic acid molecules can be joined into a one larger molecule such that each strand of the larger molecule is covalently joined (e.g., the larger molecule has no nicks). A first double-stranded nucleic acid molecule having a topoisomerase linked to each of the 5′ terminus and 3′ terminus of one end may be contacted with a second nucleic acid under conditions causing the linkage of both strands of the first nucleic acid molecule to both strands of the second nucleic acid molecule. The end of the first nucleic acid molecules to which the topoisomerases are attached may have either a 5′-overhang, 3′-overhang or be blunt ended. The end of the second nucleic acid molecule to be joined to the first nucleic acid molecule may have the same type of end as the topoisomerase-linked end of the first nucleic acid molecule. The end of the second molecule that is not to be joined may have a different end if directional joining of the segments is desired and may have the same type of end if directionality is not required.

In another embodiment, a first nucleic acid molecule having a topoisomerase bound to the 3′ terminus of one end, and a second nucleic acid molecule having a topoisomerase bound to the 3′ terminus of one end may be joined using the methods of the invention. A covalently linked double-stranded recombinant nucleic acid molecule is generated by contacting the ends containing the topoisomerase-charged substrate nucleic acid molecules. Either or both of the first and second nucleic acid molecules may comprise all or a portion of one or more Ter sites.

TA cloning. As used herein “TA cloning” is a method of cloning a nucleic acid of interest, typically a PCR product, into a cloning vector. The method takes advantage of the terminal transferase activity of some DNA polymerases such as Taq polymerase. This enzyme adds a single, 3′-A overhang to each end of the PCR product. A linear vector can be prepared that has a complementary 3′-T overhang, for example, by treatment with a nucleotidyl transferase in the presence of dTTP. The PCR product can be cloned directly into the linearized cloning vector with 3′-T overhangs using a ligase. The PCR fragment may also be cloned into the linear vector by incorporating a topoisomerase site into PCR fragment and/or the vector and using a topisomerase in conjunction with or in place of a ligase. DNA polymerases with proofreading activity, such as Pfu polymerase, can not be used because they provide blunt-ended PCR products.

Selectable marker: As used herein, a “selectable marker” is a DNA segment that allows one to select for or against a molecule (e.g., a replicon) or a cell that contains it, or to identify the presence or absence of a particular molecule, often under particular conditions. These markers can encode an activity, such as, but not limited to, production of RNA, peptide, or protein, or can provide a binding site for RNA, peptides, proteins, inorganic and organic compounds or compositions and the like. Examples of Selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product; (4) DNA segments that encode products which can be readily identified (e.g., phenotypic markers such as 3-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate or identify a desired molecule (e.g. specific protein binding sites); (9) DNA segments that encode a specific nucleotide sequence which can be otherwise non-functional (e.g., for PCR amplification of subpopulations of molecules); (10) DNA segments, which when absent, directly or indirectly confer resistance or sensitivity to particular compounds; (11) DNA segments that encode products which are toxic in recipient cells; (12) DNA segments that inhibit replication, partition or heritability of nucleic acid molecules that contain them; and/or (13) DNA segments that encode conditional replication functions, e.g., replication in certain hosts or host cell strains or under certain environmental conditions (e.g., temperature, nutritional conditions, etc.).

In some embodiments, a selectable marker may be a DNA segment encoding a toxic product. Examples of such toxic gene products are well known in the art, and include, but are not limited to, restriction endonucleases (e.g., DpnI), apoptosis-related genes (e.g. ASK1 or members of the bcl-2/ced-9 family), retroviral genes including those of the human immunodeficiency virus (HIV), defensins such as NP-1, inverted repeats or paired palindromic DNA sequences, bacteriophage lytic genes such as those from MX174 or bacteriophage T4; antibiotic sensitivity genes such as rpsL, antimicrobial sensitivity genes such as pheS, plasmid killer genes, eukaryotic transcriptional vector genes that produce a gene product toxic to bacteria, such as GATA-1, and genes that kill hosts in the absence of a suppressing function, e.g., kicB, ccdB, MX174 E (Liu, Q. et al., Curr. Biol. 8:1300-1309 (1998)), and other genes that negatively affect replicon stability and/or replication. A toxic gene can alternatively be selectable in vitro, e.g., a restriction site.

Many genes coding for restriction endonucleases operably linked to inducible promoters are known, and may be used in the present invention. See, e.g. U.S. Pat. No. 4,960,707 (DpnI and DpnII); U.S. Pat. Nos. 5,000,333, 5,082,784 and 5,192,675 (KpnI); U.S. Pat. No. 5,147,800 (NgoAIII and NgoAI); U.S. Pat. No. 5,179,015 (FspI and HaeIII): U.S. Pat. No. 5,200,333 (HaeII and TaqI); U.S. Pat. No. 5,248,605 (HpaII); U.S. Pat. No. 5,312,746 (ClaI); U.S. Pat. Nos. 5,231,021 and 5,304,480 (XhoI and XhoII); U.S. Pat. No. 5,334,526 (AluI); U.S. Pat. No. 5,470,740 (NsiI); U.S. Pat. No. 5,534,428 (Ssti/SacI); U.S. Pat. No. 5,202,248 (NcoI); U.S. Pat. No. 5,139,942 (NdeI); and U.S. Pat. No. 5,098,839 (Pad). See also Wilson, G. G., Nucl. Acids Res. 19:2539-2566 (1991); and Lunnen, K. D., et al., Gene 74:25-32 (1988). Ter sites.

Ter sites according to the invention are any replication termination sequence from any source including those found in eukaryotic and prokaryotic organisms (including gram positive, gram negative, mesophilic and thermophilic microorganisms). The invention also contemplates any portion of such Ter sites that may be recognized and bound by one or more Ter-binding proteins such as replication terminator proteins or peptides. A portion of a Ter site may comprise from about 6, 7, 8 or more nucleotides of a Ter site but less than an entire site. In some aspects, a Ter site may comprise a double-stranded nucleic acid composition, e.g., a double-stranded molecule one strand of which comprises a sequence listed in Table 4 and the other strand having a sequence complementary to the first strand, or a single stranded nucleic acid comprising a sequence from Table 4 or a single stranded molecule comprising a sequence complementary to a sequence in Table 4. The invention is also directed to mutant or derivative Ter sites (and portions and combinations thereof) that have the same, increased or decreased ability to be bound by such Ter-binding proteins or peptides. Mutant or derivative Ter sites for use in the invention may be made by standard mutagenesis techniques (to make deletions, substitutions and insertions in the sequence of interest) or desired derivative Ter sites may be made by standard chemical synthesis techniques (e.g., oligonucleotide synthesis). Ter sites for use in the invention have been identified in a variety of organisms and plasmids. Table 4 presents the nucleotide sequences of a representative number of sites from E. coli and related species as well as plasmids and a number of Bacillus species.

TABLE 4

| E. coli | | |
|---|---|---|
| TerA | AATTA GTATG TTGTA ACTAA AGT | (SEQ ID NO: 1) |
| TerB | AATAA GTATG TTGTA ACTAA AGT | (SEQ ID NO: 2) |
| TerC | ATATA GGATG TTGTA ACTAA TAT | (SEQ ID NO: 3) |
| TerD | CATTA GTATG TTGTA ACTAA ATG | (SEQ ID NO: 4) |
| TerE | TTAAA GTATG TTGTA ACTAA G | (SEQ ID NO: 5) |
| TerF | CCTTC GTATG TTGTA ACGAC GAT | (SEQ ID NO: 6) |
| TerG | GATGA GTATG TTGTA ACTAA CTA | (SEQ ID NO: 7) |
| TerH | CGATC GTATG TTGTA ACTAT CTC | (SEQ ID NO: 68) |
| TerI | AACAT GTATG TTGTA ACTAA CCG | (SEQ ID NO: 69) |
| TerJ | ACGCA GTAAG TTGTA ACTAA TGC | (SEQ ID NO: 70) |
| S. typhimurium | | |
| TerA | ATTAA GTATG TTGTA ACTAA AGC | (SEQ ID NO: 8) |
| Ter(amyA) | GATGA GTATG TTGTA ACTAA ATG | (SEQ ID NO: 9) |
| Plasmids | | |
| R6KterR1 | CTCTT GTGTG TTGTA ACTAA ATC | (SEQ ID NO: 10) |
| R6KterR2 | CTATT GAGTG TTGTA ACTAC TAG | (SEQ ID NO: 11) |
| R100TerR1 | ATTAT GAATG TTGTA ACTAC TTC | (SEQ ID NO: 12) |
| R100TerR2 | TGTCT GAGTG TTGTA ACTAA AGC | (SEQ ID NO: 13) |
| R1TerR1 | ATTAT GAATG TTGTA ACTAC ATC | (SEQ ID NO: 14) |
| R1TerR2 | TTTTT GTGTG TTGTA ACTAA ATT | (SEQ ID NO: 15) |
| RepFICTerR1 | ATTAT GAATG TTGTA ACTAC ATT | (SEQ ID NO: 16) |
| St90kbTer | ATTTT GGATG TTGTA ACTAT TTG | (SEQ ID NO: 17) |
| Bacillus spp. | | |
| B. atrophaeus | | |
| TerI | GAACT AAATA AACTA TGTAC CAAAT GTTCA | (SEQ ID NO: 18) |
| TerII | TAACT GAAAA CACTA TGTAC TAAAT ATTCA | (SEQ ID NO: 19) |
| B. mojavensis | | |
| TerI | GAACA AAACA AACTA TGTAC CAAAT GTTCA | (SEQ ID NO: 20) |
| TerII | AAACT GAGAA TACTA TGTAC TAAAT ATTCA | (SEQ ID NO: 21) |
| B. vallismortis | | |
| TerII | ATACT AAAAA TATGA TGTAC TAAAT ATTCA | (SEQ ID NO: 22) |
| B. amyloliquefaciens | | |
| TerII | TAACA AATTA TTCCA TGTAC TAAAT ATTCT | (SEQ ID NO: 23) |
| B. subtilis 168 | | |
| TerVIII | GAACT AATTA AACTA TGTAC TAAAT TTTCA | (SEQ ID NO: 24) |
| TerIX | ATACT AATTG ATCCA TGTAC TAAAT TTTCA | (SEQ ID NO: 25) |

The nucleotide sequences of the various Ter sites presented in Table 4 indicate that certain positions are highly conserved. In E. coli the G at residue 6 and the 11 bases starting with position 8 and ending with position 19 are conserved in all Ter sites with the sole exception of a T/G modification at position 18 of the TerF sequence. In Bacillus nucleotides 3-5, 7, 13, 15, 16-20, and 22-25 of the sequences in Table 4 are highly conserved.

The present invention contemplates the use of Ter sites and Ter-binding proteins from any source. In some embodiments, the Ter sites and Ter-binding proteins may be derived from prokaryotes, for example, thermophilic organisms such as, for example, B. stearothermophilus. Other source organisms from which thermophilic or mesophilic Ter-binding proteins and their corresponding Ter sites may be isolated and used in the practice of the invention include, but are not limited to, Thermus thermophilus, Thermus aquaticus, Thermotoga neopolitana, Thermotoga maritima, Thermococcus litoralis, Pyrococcus furiosus, Pyrococcus woosii, Bacillus sterothermophilus, Sulfolobus acidocaldarius (Sac), Thermoplasma acidophilum, Thermus flavus, Thermus ruber, Thermus brockianus, and Methanobacterium thermoautotrophicum. Other sources include Enterobacteriaceae, species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, Xanthomonas and Streptomyces.

Ter sites that have been altered by removing a portion of the sequence or by substitution or mutation and that still (1) retain the ability to bind Ter-binding protein are included as part of this invention and/or (2) still retain directionality are included as part of this invention. Functional domains and regions of Ter sites necessary for proper function are described in Coskun-Ari and Hill, J. Biol. Chem. 17 272:26448-26456 (1997). Ter sites that are altered such that a Ter-binding protein binds with less affinity are also useful in reactions where, for example, manipulation of replication termination is desired (Coskun-Ari and Hill, 1997; Sharma and Hill, Mol. Microbiol. 18:45-61 (1995)).

The present invention also contemplates the use of Ter sites having at least about 75%, 80%, 85%, 90%, 95%, or 99% sequence identity to one or more of the sequences in Table 4 and that retain the ability to be bound by one or more Ter-binding proteins.

As a practical matter, whether any particular nucleic acid molecule is at least 75%, 80%, 85%, 90%, 95%, 96%, 97%, 98% or 99% identical to, for instance, a given Ter site nucleotide sequence or portion thereof can be determined conventionally using known computer programs such as DNAsis software (Hitachi Software, San Bruno, Calif.) for initial sequence alignment followed by ESEE version 3.0 DNA/protein sequence software (cabot@trog.mbb.sfu.ca) for multiple sequence alignments. Alternatively, such determinations may be accomplished using the BESTFIT program (Wisconsin Sequence Analysis Package, Genetics Computer Group, University Research Park, 575 Science Drive, Madison, Wis. 53711), which employs a local homology algorithm (Smith and Waterman, Advances in Applied Mathematics 2: 482-489 (1981)) to find the best segment of homology between two sequences. When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed. Computer programs such as those discussed above may also be used to determine percent identity and homology between two proteins at the amino acid level.

Nucleic acids comprising the Ter sites of the invention may be prepared using any convention technology, for example, chemical synthesis using phosphoramidite chemistry or amplification techniques, i.e., PCR and the like. Optionally, detectable molecules may be attached to the nucleic acids comprising the Ter sites. Suitable detection molecules are known to those skilled in the art and include, but are not limited to, enzymes such as horseradish peroxidase, alkaline phosphatase, luciferase, beta-galactosidase and beta-glucuronidase, fluorescent moieties, chromophores, haptens and/or epitopes recognized by an antibody. Detection molecules may be attached during synthesis, for example, by using chemically modified nucleotides—for example, fluorescently labeled—during an amplification reaction. In some instances it may be desirable to introduce a detection molecule after synthesis of the nucleic acid, for example, by chemically coupling the detection molecule to the nucleic acid.

Oligonucleotides comprising Ter sites may be single or double stranded. In some embodiments, oligonucleotides may be in the form of a hairpin or stem-loop such that one portion of the oligonucleotide hybridizes to another portion of the oligonucleotide to form a double stranded portion of the oligonucleotide comprising all or a portion of a Ter site.

### Ter-Binding Proteins.

In one aspect, the present invention also contemplates proteins that bind to the Ter sites of the invention. Ter-binding proteins of the invention include, but are not limited to, wild-type Ter-binding proteins, mutants of wild-type Ter-binding proteins (e.g., point mutants, truncation mutants, insertion mutants, and combinations thereof), fragments of Ter-binding proteins that retain the ability to bind with a Ter-site of the invention, and combinations thereof (e.g., fragments of mutants). Ter-binding proteins of the invention also include chimeric proteins comprising all or a portion of two or more Ter-binding proteins that may be the same or different. By way of non-limiting example, a chimeric Ter-binding protein could comprise amino acid residues 1-90 of a S. typhimurium Ter-binding protein (Table 7) and 91-310 of K. pneumoniae Ter-binding protein (Table 10). Note that amino acid residues 71-90 are identical in both proteins. Ter-binding proteins of the present invention also comprise fusion proteins having one or more Ter-binding portions (i.e., wild-type, mutant, and/or fragment as described above) and one or more additional polypeptide portions. Ter-binding proteins of the invention also included modified Ter-binding proteins, for example, a Ter-binding protein (e.g., wild-type, mutant, fusion and/or fragment) comprising one or more modifying groups (e.g., labels, haptens, detectable moieties, and the like). Modifying groups may be directly or indirectly, covalent or non-covalently attached or bound to Ter-binding proteins of the invention. Ter-binding proteins of the invention may comprise combinations of the above-described characteristics. For example, a Ter-binding protein of the invention may include one or more Ter-binding portions (e.g., wild-type, mutant, and/or fragments thereof), one or more additional polypeptide portions (i.e., fusions) and/or one or more modifying groups (e.g., detectable moieties, labels, etc.).

One example of a Ter-binding protein is a replication terminator protein (RTP). An RTP is a sequence specific DNA-binding protein which, when bound to the double stranded termination sequence, allows replication arrest. The RTP from E. coli is a 36,000 Da protein designated Tus (also tau). The Tus protein binds Ter sites as a monomer. Tus binds the TerB site extremely tightly with a dissociation constant of up to 3×10⁻¹³ M in vitro (depending on the buffer conditions). The binding of Tus to other Ter sites is somewhat less tight with dissociation constants on the order of 10⁻¹⁰ to 10¹¹ M. Preferred Ter-binding proteins of the present invention may have a dissociation constant from a Ter site of from about 10⁻⁹ M to about 10⁻¹⁵ M, from about 10⁻¹° M to about 10⁻¹⁴ M, or from about 10⁻¹¹ M to about 10⁻¹³ M.

The amino acid sequences of some representative Ter-binding proteins are provided in Tables 5-13.

TABLE 5
Amino acid sequence of E. coli K-12 Ter-binding protein
(GenBank accession no. AAC74682) (SEQ ID NO: 71)

| | |
|---|---|
| 1 | marydlvdrl nttfrqmeqe laifaahleq hkllvarvfs lpevkkedeh nplnrievkq |
| 61 | hlgndaqsla lrhfrhlfiq qqsenrsska avrlpgvlcy qvdnlsqaal vshiqhinkl |
| 121 | kttfehivtv eselptaarf ewvhrhlpgl itlnayrtlt vlhdpatlrf gwankhiikn |
| 181 | lhrdevlaql ekslksprsv apwtreewqr klereyqdia alpqnaklki krpvkvqpia |
| 241 | rvwykgdqkq vqhacptpli alinrdngag vpdvgellny dadnvqhryk pqaqplrlii |
| 301 | prlhlyvad |

TABLE 6
Amino acid sequence of E. coli O157:H7 Ter-binding protein
(GenBank accession number NP_310343) (SEQ ID NO: 72)

| | |
|---|---|
| 1 | marydlvdrl nttfrqmeqe laafaahleq hkllvarvfs lpevkkedeh nplnrievkq |
| 61 | hlgndaqsqa lrhfrhlfiq qqsenrsska avrlpgvlcy qvdnlsqaal vshiqhinkl |
| 121 | kttfehivtv eselptaarf ewvhrhlpgl itlnayrtlt vlhdpatlrf gwankhiikn |
| 181 | lhrdevlaql ekslksprsv apwtreewqr klereyqdia alpqnaklki krpvkvqpia |
| 241 | rvwykgdqkq vqhacptpli alinrdngag vpdvgellny dadnvqhryk pqaqplrlii |
| 301 | prlhlyvad |

TABLE 7
Amino acid sequence of Salmonella typhimurium LT2 Ter-
binding protein (GenBank accession number AAL20390) (SEQ ID NO: 73)

| | |
|---|---|
| 1 | msrydlverl ngtfrqieqh laaltdnlqq hslliarvfs lpqvtkeaeh apldtievtq |
| 61 | hlgkeaeala lrhyrhlfiq qqsenrsska avrlpgvlcy qvdnatqldl enqiqrinql |
| 121 | kttfeqmvtv esglpsaarf ewvhrhlpgl itlnayrtlt linnpatirf gwankhiikn |
| 181 | lsrdevlsql kkslasprsv ppwtreqwqf klereyqdia alpqqarlki krpvkvqpis |
| 241 | riwykgqqkq vqhacptpii alintdngag vpdiggleny dadniqhrfk pqaqplrlii |
| 301 | prlhlyvad |

TABLE 8
Amino acid sequence of Salmonella typhi Ter-binding protein
(GenBank accession number Q8Z6R7) (SEQ ID NO: 74)

| | |
|---|---|
| 1 | msrydlverl ngtfrqieqh laalsdnlqq hslliasvfs lpqvtkeaeh apldtievtq |
| 61 | hlgkeaeala lrhyrhlfiq qqsenrsska avrlpgvlcy qvdnatqldl enqvqrinql |
| 121 | kttfeqmvtv esglpsaarf ewvhrhlpgl itlnayrtlt linnpatirf gwankhiikn |
| 181 | lsrdevlsql kkslasprsv ppwtreqwqf klereyqdia alpqqaklki krpvkvqpia |
| 241 | riwykgqqkq vqhacpspii alintdngag vpdiggleny dadniqhrfk pqaqplrlii |
| 301 | prlhlyvad |

TABLE 9
Amino acid sequence of Salmonella enterica subsp. enterica
serovar Typhi Ter-binding protein
(GenBank accession number NP_456062) (SEQ ID NO: 75)

| | |
|---|---|
| 1 | msrydlverl ngtfrqieqh laalsdnlqq hslliasvfs lpqvtkeaeh apldtievtq |
| 61 | hlgkeaeala lrhyrhlfiq qqsenrsska avrlpgvlcy qvdnatqldl enqvqrinql |
| 121 | kttfeqmvtv esglpsaarf ewvhrhlpgl itlnayrtlt linnpatirf gwankhiikn |
| 181 | lsrdevlsql kkslasprsv ppwtreqwqf klereyqdia alpqqaklki krpvkvqpia |
| 241 | riwykgqqkq vqhacpspii alintdngag vpdiggleny dadniqhrfk pqaqplrlii |
| 301 | prlhlyvad |

TABLE 10
Amino acid sequence of Klebsiella pneumoniae subsp.
ozaenae Ter-binding protein (GenBank accession number O52715)
(SEQ ID NO: 76)

| | |
|---|---|
| 1 | masydlverl nntfrqiele lqalqqalsd crllagrvfe lpaigkdaeh dplatipvvq |
| 61 | higktalara lrhyshlfiq qqsenrsska avrlpgaicl qvtaaeqqdl lariqhinal |
| 121 | katfekivtv dsglpptarf ewvhrhlpgl itlsayrtlt plvdpstirf gwankhvikn |
| 181 | ltrdqvlmml ekslqaprav ppwtreqwqs klereyqdia alpqrarlki krpvkvqpia |
| 241 | rvwyageqkq vqyacpspli almsgsrgvs vpdigellny dadnvqyryk peaqslrlli |
| 301 | prlhlwlase |

TABLE 11
Amino acid sequence of Proteus vulgaris Ter-binding
protein (GenBank accession number NP_640052) (SEQ ID NO: 77)

| | |
|---|---|
| 1 | mdlkktfeql tddllalkml isgssplfsq vsdippvlrg dehlpisyva pdhlygheai |
| 61 | qkavdiwsdl hikhdfsqks arrasgvlwf psednaftve lvrllsqina lkksiethii |
| 121 | ttyqtrsarf ealhnqcagv ltlhlyrqir wwkdehisav rfswqekesl lipdkaellv |
| 181 | rmskegredg kkevplallm kqivsvpeer lrirrrlkvq psanisfrse qhptgkltmv |
| 241 | tapmpfiiiq nerpevkmlk iydanerisr krrndkvhte ilgtfhgesi evia |

TABLE 12
Amino acid sequence of Bacillus subtilis Ter-binding protein
(GenBank accession number A32807) (SEQ ID NO: 78)

| | |
|---|---|
| 1 | mkeekrsstg flvkqraflk lymitmteqe rlyglkllev lrsefkeigf kpnhtevyrs |
| 61 | lhellddgil kqikvkkega klqevvlyqf kdyeaaklyk kqlkveldrc kkliekalsd |
| 121 | nf |

TABLE 13
Amino acid sequence of Yersinia pestis Ter-binding protein
(GenBank accession number NP_405802) (SEQ ID NO: 79)

| | |
|---|---|
| 1 | mnkydlierm ntrfaelevt lhqlhqqldd lpliaarvfs lpeiekgteh qpieqitvni |
| 61 | tegehakklg lqhfqrlflh hqgqhvsska alrlpgvlcf svtdkeliec qdiikktnql |
| 121 | kaelehiitv esglpseqrf efvhthlhgl itlntyrtit plinpssvrf gwankhiikn |
| 181 | vtredillql ekslnagrav ppftreqwre lisleindvq rlpektrlki krpvkvqpia |
| 241 | rvwyqeqqkq vqhpcpmpli afcqhqlgae lpklgeltdy dvkhikhkyk pdakplrllv |
| 301 | prlhlyvele p |

TABLE 14
Amino acid sequence of IncT plasmid R394 Ter-binding
protein (GenBank accession number AAG33668.1) (SEQ ID NO: 80)

| | |
|---|---|
| 1 | mdlkktfeql tddllalkml isgssplfsq vsdippvlrg dehlpisyva pdhlygheai |
| 61 | qkavdiwsdl hikhdfsqks arrasgvlwf psednaftve lvrllsqina lkksiethii |
| 121 | ttyqtrsarf ealhnqcagv ltlhlyrqir wwkdehisav rfswqekesl lipdkaellv |
| 181 | rmskegredg kkevplallm kqivsvpeer lrirrrlkvq psanisfrse qhptgkltmv |
| 241 | tapmpfiiiq nerpevkmlk iydanerisr krrndkvhte ilgtfhgesi evia |

The Tus-TerB complex is very stable with a half-life of up to 550 minutes. The DNA sequence of the Tus gene is known (see, Hidaka, M., et al., Purification of a DNA replication terminus (ter) site-binding protein in Escherichia coli and identification of the structural gene, J. Biol. Chem. 264 (35):21031-21037 (1989) and Hill, T. M., et al., Tus, the trans-acting gene required for termination of DNA replication in Escherichia coli, encodes a DNA-binding protein, Proc. Natl. Acad. Sci. U.S.A. 86 (5):1593-1597 (1989)). Strains of E. coli that lack functional Tus protein are known (e.g., Dasgupta, et al., Res Microbiol 142(2-3):177-80, 1991, Skokotas, et al., J Biol. Chem. 270(52):30941-8, 1995, Skokotas, et al., J Biol. Chem. 69(32):20446-55, 1994, Henderson et al., Mol Genet Genomics 265(6):941-53, 2001, and Sharma et al., Mol Microbiol 18(1):45-61, 1995). The crystal structure of the protein in a complex with a Ter site has been produced (Bussiere, et al., Molecular Microbiology 31(6): 1611-1618 (1999)).

Mutants and variants of Ter-binding proteins still able to bind, or with altered ability to bind, for use in certain applications are part of the present invention. Such mutants include those with mutations in the DNA-binding domain such as those that correspond to mutations in amino acids E49, H50, K89, T136, K175, I177, R198, R232, V234, K235, Q237, Q252, A254, R288, K290 of the E. coli replication termination protein (Skokotas et al., J. Biol. Chem. 270:30941-30948 (1995)). Functional domains of some Ter-binding proteins have been defined and may be altered to increase or decrease its ability to bind Ter, for example, mutants in the replication fork blocking domain such as those that correspond to mutations in amino acids H31, K32, L33, L34, V35, A36, R37, L62, V97, L98, C99, Y100, Q101, V102, D103, N104, S106, Q107, L110, V161, L162, H136, D164, P165,A166, T167, L168, R169, F170, 8241, V242, W243, Y244, K245, G246, D247, Q248, L259, 1260, A261, L262, N264, R265, D266, N267, G268, A269, G270, V271, P272, D273, V274, G275 of the E. coli RTP (Duggin et al, J. Mol. Biol. 286:1325-1335 (1999)). One skilled in the art can identify amino acids in other RTPs that correspond to those identified above by aligning the sequences of other RTPs to those RTPs identified above. Such alignments may be accomplished using standard homology searching programs (e.g., BLAST) by routine experimentation.

Ter-binding proteins of the invention further comprise polypeptides which are 70%, 75%, 80%, 85%, 90%, 95%, 96%, 97%, 98%, or 99% identical to one or more known Ter-binding proteins. Preferably such polypeptides retain the ability to specifically bind a Ter site.

By a protein or protein fragment having an amino acid sequence at least, for example, 70% “identical” to a reference amino acid sequence it is intended that the amino acid sequence of the protein is identical to the reference sequence except that the protein sequence may include up to 30 amino acid alterations per each 100 amino acids of the amino acid sequence of the reference protein. In other words, to obtain a protein having an amino acid sequence at least 70% identical to a reference amino acid sequence, up to 30% of the amino acid residues in the reference sequence may be deleted or substituted with another amino acid, or a number of amino acids up to 30% of the total amino acid residues in the reference sequence may be inserted into the reference sequence. These alterations of the reference sequence may occur at the amino (N-) and/or carboxy (C-) terminal positions of the reference amino acid sequence and/or anywhere between those terminal positions, interspersed either individually among residues in the reference sequence and/or in one or more contiguous groups within the reference sequence. As a practical matter, whether a given amino acid sequence is, for example, at least 70% identical to the amino acid sequence of a reference protein can be determined conventionally using known computer programs such as those described above for nucleic acid sequence identity determinations, or using the CLUSTAL W program (Thompson, J. D., et al., Nucleic Acids Res. 22:4673-4680 (1994)).

Sequence identity may be determined by comparing a reference sequence or a subsequence of the reference sequence to a test sequence. The reference sequence and the test sequence are optimally aligned over an arbitrary number of residues termed a comparison window. In order to obtain optimal alignment, additions or deletions, such as gaps, may be introduced into the test sequence. The percent sequence identity is determined by determining the number of positions at which the same residue is present in both sequences and dividing the number of matching positions by the total length of the sequences in the comparison window and multiplying by 100 to give the percentage. In addition to the number of matching positions, the number and size of gaps is also considered in calculating the percentage sequence identity.

Sequence identity is typically determined using computer programs. A representative program is the BLAST (Basic Local Alignment Search Tool) program publicly accessible at the National Center for Biotechnology Information (NCBI, http://www.ncbi.nlm.nih.gov/). This program compares segments in a test sequence to sequences in a database to determine the statistical significance of the matches, then identifies and reports only those matches that that are more significant than a threshold level. A suitable version of the BLAST program is one that allows gaps, for example, version 2.X (Altschul, et al., Nucleic Acids Res. 25(17):3389-402, 1997). Standard BLAST programs for searching nucleotide sequences (blastn) or protein (blastp) may be used. Translated query searches in which the query sequence is translated, i.e., from nucleotide sequence to protein (blastx) or from protein to nucleic acid sequence (tbblastn) may also be used as well as queries in which a nucleotide query sequence is translated into protein sequences in all 6 reading frames and then compared to an NCBI nucleotide database which has been translated in all six reading frames (tbblastx).

Additional suitable programs for identifying proteins with sequence identity to the proteins of the invention include, but are not limited to, PHI-BLAST (Pattern Hit Initiated BLAST, Zhang, et al., Nucleic Acids Res. 26(17):3986-90, 1998) and PSI-BLAST (Position-Specific Iterated BLAST, Altschul, et al., Nucleic Acids Res. 25(17):3389-402, 1997).

Programs may be used with default searching parameters. Alternatively, one or more search parameter may be adjusted. Selecting suitable search parameter values is within the abilities of one of ordinary skill in the art.

In some embodiments, modified Ter-binding proteins may include a cyclized Ter-binding protein, which is resistant to denaturation (e.g., by chemicals and/or heat). Such Ter-binding proteins may be used to prevent duplex DNA from denaturing under conditions (e.g., pH, ionic strength, temperature, etc.) that normally result in duplex denaturation. The cyclized protein can further be labeled to detect double stranded nucleic acid.

Also included are Ter-binding proteins that are derived from thermostable organisms as well as those derived from hypothermophiles or psychrophiles.

The present invention also comprises modified Ter-binding proteins. The modified Ter-binding protein may be a full length Ter-binding protein (e.g., wild-type or mutant) or a portion of a Ter-binding protein (e.g., wild-type or mutant) that retains the ability to bind a Ter site. The modifying moieties may be covalently attached to the Ter-binding protein, for example, by coupling using those coupling reagents known to those skilled in the art. Suitable coupling reagents are commercially available from, for example, Pierce Chemical Co., Rockford, Ill.

In some embodiments, the modifying moiety may be a polypeptide and the peptide backbone of the polypeptide may be contiguous with the peptide backbone of the Ter-binding protein forming a fusion protein between the Ter-binding protein and one or more modifying polypeptides. The construction of fusion proteins is routine in the art. One or more suitable polypeptides may be fused to all or a portion of a Ter-binding protein. The polypeptides may be fused at the N-terminal of the Ter-binding protein, the C-terminal of the Ter-binding protein and/or at an interior position of the Ter-binding protein. In some embodiments, more than one polypeptide may be fused to a Ter-binding protein and such polypeptides may be the same or different. Any site of fusion may be used so long as the binding capability of the Ter-binding protein is not substantially reduced. In this context, substantially reduced indicates that the modified Ter-binding protein does not bind a Ter site with sufficient affinity to allow detection of the modified Ter-binding protein.

Any desired modifying group may be attached to a Ter-binding protein for use in the present invention by chemical coupling and/or by preparation of a fusion protein. In some embodiments, the modifying group may be a ligand for a receptor. Ligands for use in the present invention may be ligands for cell surface receptors including, but not limited to, the transferrin receptor, the serum albumin receptor, the asialoglycoprotein receptor, an adenovirus receptor, a retrovirus receptor, CD4, lipoprotein (a) receptor, immunoglobulin Fc receptor, α-fetoprotein receptor, LDLR-like protein (LRP) receptor, acetylated LDL receptor, mannose receptor, or mannose-6-phosphate receptor. Many other cell surface receptors and their associated ligands are known to those skilled in the art and modified Ter-binding proteins comprising these ligands are within the scope of the present invention. For a detailed list of receptors and ligands and their use to transport molecules into cells see U.S. Pat. No. 6,331,289, issued to Klaveness, et al., and U.S. Pat. No. 6,262,026, issued to Heartlein, et al. A modified Ter-binding protein comprising a ligand for a cell surface receptor can be used as a means by which nucleic acids comprising a Ter site can be transported into cells. Proteins comprising a Ter-binding protein and a ligand for one or more receptors may be contacted with a nucleic acid comprising a Ter site in order to form a complex of nucleic acid-Ter-binding protein-ligand. The complex may then be brought into contact with a cell expressing the appropriate receptor resulting in the up take of the complex into the target cell. Suitable receptors are present on a wide variety of different cell types and allow uptake of nucleic acids comprising a Ter site into a wide variety of cell types.

In some embodiments, a Ter-binding protein may comprise a detection molecule. Suitable detection molecules are known to those skilled in the art and include, but are not limited to, enzymes with detectable activities such as horse radish peroxidase, alkaline phosphatase, luciferase, beta-galactosidase and beta-glucuronidase, fluorescent moieties, chromophores, haptens and/or epitopes recognized by an antibody. In some preferred embodiments, the detection molecule may comprise combinations of fluorescent moieties, chromophores, enzymes, haptens and/or epitopes and the like. Detection molecules may be covalently attached to a Ter-binding protein by chemical coupling and/or by construction of a fusion protein.

In some embodiments, the modified Ter-binding proteins of the present invention may comprise a cellular targeting sequence. Such a sequence directs the Ter-binding protein and any nucleic acid bound by the protein to one or more specific locations in an organism or cell. Vectors comprising targeting signals are commercially available, for example, pSHOOTER™ available from Invitrogen Corporation, Carlsbad, Calif. In some embodiments, the cellular targeting sequence may be a nuclear localization sequence (e.g., SV 40 large T antigen heptapeptide: Pro Lys Lys Lys Arg Lys Val (SEQ ID NO:81), the influenza virus nucleoprotein decapeptide: Ala Ala Phe Glu Asp Leu Arg Val Leu Ser (SEQ ID NO:82), and the adenovirus E1a protein sequence: Lys Arg Pro Arg Pro (SEQ ID NO:83)) and the Ter-binding protein and bound nucleic acid may be directed to the nucleus of a target cell. Other sequences may be found in C. Dingwall, et al., TIBS 16:478-481, (1991).

Cellular targeting sequences may also help reduce or prevent degradation of the nucleic acid molecule, for example, degradation occurring in the endosomes and/or lysomes. Suitable cellular targeting sequences are known to those skilled in the art and may be derived from any source, for example, from viral proteins. For examples of suitable cellular targeting sequences as well as examples of suitable ligands and other polypeptide portions that may be used to modify the Ter-binding proteins of the invention, see U.S. Pat. No. 6,177,554, issued to Woo, et al.

In some embodiments, a cellular targeting sequence may target a cellular location other than the nucleus. For example, a cellular targeting sequence may direct a molecule to which it is attached to ribosomes, mitochondria, and chloroplasts. In an embodiment of this invention, a cellular targeting sequence may be a lysosomal targeting sequence (e.g., Lys Phe Glu Arg Gln (SEQ ID NO:84)). In yet another embodiment, the cellular targeting sequence may be a mitochondrial targeting sequence (e.g., Met Leu Ser Leu Arg Gln Ser Ile Arg Phe Phe Lys Pro Ala Thr Arg (SEQ ID NO:85)). Other suitable targeting sequences are known to those skilled in the art and may be used in the practice of the present invention, for example, those found in U.S. Pat. No. 6,300,317, issued to Szoka, et al.

In some embodiments, the present invention provides a fusion protein comprising a Ter-binding protein and a polypeptide or protein of interest. The presence of the Ter-binding protein permits the detection and/or affinity purification of the polypeptide or protein of interest using an oligonucleotide comprising a Ter site. For example, an oligonucleotide comprising a Ter site may be attached to a support, for example, a bead, a chromatography support and the like. The fusion protein comprising a Ter-binding portion and a polypeptide of interest may then be contacted with the support under conditions—pH, ionic strength, temperature and the like—that permit the binding of the Ter-binding portion of the fusion protein to the oligonucleotide. Any contaminating molecules may be washed from the support and the bound fusion protein may be eluted.

The fusion proteins of the present invention may optionally comprise one or more cleavage sites for proteolytic enzymes. In some embodiments, one or more cleavage sites may be located between the Ter-binding portion of the fusion protein and one or more additional polypeptide portions. The construction of fusion proteins comprising cleavage sites is well known in the art, see, for example, Riggs, et al., in Current Protocols in Molecular Biology, Ausubel, et al. Eds., John Wiley & Sons, Inc. Chapter 16, pages 16.4.1-16.4.4, 1997. In embodiments of this type, one or more amino acids forming a cleavage site, e.g., for a protease enzyme, may be incorporated into the primary sequence of the fusion protein. The cleavage site may be located such that cleavage at the site may remove all or a portion of an exogenous polypeptide sequence from the Ter-binding protein. Examples of suitable cleavage sites include, but are not limited to, the Factor Xa cleavage site having the sequence Ile-Glu-Gly-Arg (SEQ ID NO:86), which is recognized and cleaved by blood coagulation factor Xa, and the thrombin cleavage site having the sequence Leu-Val-Pro-Arg (SEQ ID NO:87), which is recognized and cleaved by thrombin. Other suitable cleavage sites are known to those skilled in the art and may be used in conjunction with the present invention.

In some embodiments, the modified Ter-binding proteins of the present invention may comprise more than one (e.g., two, three, four, five, six, seven, eight, nine, ten, etc.) Ter-binding portions. When two or more Ter-binding portions are linked, they may be from the same or different Ter-binding proteins and have the same or different affinities for Ter sites. Multiple Ter-binding proteins may be linked by chemically coupling Ter-binding proteins or by the creation of fusion proteins. The multivalent Ter-binding proteins can be made by cloning—with or without linkers—direct repeats of the open reading frame encoding a Ter-binding protein or by crosslinking the two molecules, for example. Modified Ter-binding proteins comprising multiple Ter-binding portions may also further comprise additional modifications, for example, detection molecules, ligands and other modifications.

In some embodiments, a Ter-binding protein may comprise more than one modification. For example, a Ter-binding protein of the invention (e.g., wild-type, mutant, and/or fragment thereof) may comprise a ligand for a cell surface receptor and a detection molecule. A configuration of this sort will allow detection of the uptake of the modified Ter-binding protein, preferably provide the ability to detect a complex of the modified Ter-binding protein and a nucleic acid to which it is bound. In some embodiments, Ter-binding proteins of the invention may comprise a plurality of modifications (e.g., two, three, four, five, six, seven, eight, nine, ten, etc.), which may be the same or different.

### Polymerases

Preferred polypeptides having reverse transcriptase activity (i.e., those polypeptides able to catalyze the synthesis of a DNA molecule from an RNA template) include, but are not limited to Moloney Murine Leukemia Virus (M-MLV) reverse transcriptase, Rous Sarcoma Virus (RSV) reverse transcriptase, Avian Myeloblastosis Virus (AMV) reverse transcriptase, Rous Associated Virus (RAV) reverse transcriptase, Myeloblastosis Associated Virus (MAV) reverse transcriptase, Human Immunodeficiency Virus (HIV) reverse transcriptase, retroviral reverse transcriptase, retrotransposon reverse transcriptase, hepatitis B reverse transcriptase, cauliflower mosaic virus reverse transcriptase and bacterial reverse transcriptase. Particularly preferred are those polypeptides having reverse transcriptase activity that are also substantially reduced in RNAse H activity (i.e., ARNAse H⁻@ polypeptides). By a polypeptide that is Asubstantially reduced in RNase H activity@ is meant that the polypeptide has less than about 20%, more preferably less than about 15%, 10% or 5%, and most preferably less than about 2%, of the RNase H activity of a wildtype or RNase H⁺ enzyme such as wildtype M-MLV reverse transcriptase. The RNase H activity may be determined by a variety of assays, such as those described, for example, in U.S. Pat. No. 5,244,797, in Kotewicz, M. L. et al., Nucl. Acids Res. 16:265 (1988) and in Gerard, G F., et al., FOCUS 14(5):91 (1992), the disclosures of all of which are fully incorporated herein by reference. Suitable RNAse polypeptides for use in the present invention include, but are not limited to, M-MLV H⁻ reverse transcriptase, RSV H⁻ reverse transcriptase, AMV H⁻ reverse transcriptase, RAV H⁻ reverse transcriptase, MAV H⁻ reverse transcriptase, HIV H⁻ reverse transcriptase, and SUPERSCRIPTJ I reverse transcriptase and SUPERSCRIPTJ II reverse transcriptase which are available commercially, for example from Life Technologies, Inc. (Rockville, Md.).

Other polypeptides having nucleic acid polymerase activity suitable for use in the present methods include DNA polymerases such as DNA polymerase I, DNA polymerase III, Klenow fragment, T7 polymerase, and T5 polymerase, and thermostable DNA polymerases including, but not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT7) DNA polymerase, Pyrococcus furiosus (Pfu or DEEPVENT7) DNA polymerase, Pyrococcus woosii (Pwo) DNA polymerase, Bacillus sterothermophilus (Bst) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME7) DNA polymerase, Methanobacterium thermoautotrophicum (Mth) DNA polymerase, and mutants, variants and derivatives thereof.

Production/Sources of cDNA Molecules

In accordance with the invention, cDNA molecules (single-stranded or double-stranded) may be prepared from a variety of nucleic acid template molecules. In preferred embodiments, cDNA molecules prepared according to the invention may comprise all or a portion of one or more Ter sites. Preferred nucleic acid molecules for use in the present invention include single-stranded or double-stranded DNA and RNA molecules, as well as double-stranded DNA:RNA hybrids. More preferred nucleic acid molecules include messenger RNA (mRNA), transfer RNA (tRNA) and ribosomal RNA (rRNA) molecules, although mRNA molecules are the preferred template according to the invention.

The nucleic acid molecules that are used to prepare cDNA molecules according to the methods of the present invention may be prepared synthetically according to standard organic chemical synthesis methods that will be familiar to one of ordinary skill. More preferably, the nucleic acid molecules may be obtained from natural sources, such as a variety of cells, tissues, organs or organisms. Cells that may be used as sources of nucleic acid molecules may be prokaryotic (bacterial cells, including but not limited to those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, Xanthomonas and Streptomyces) or eukaryotic (including fungi (especially yeasts), plants, protozoans and other parasites, and animals including insects (particularly Drosophila spp. cells), nematodes (particularly Caenorhabditis elegans cells), and mammals (particularly human cells)).

Mammalian somatic cells that may be used as sources of nucleic acids include blood cells (reticulocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells). Mammalian germ cells (spermatocytes and oocytes) may also be used as sources of nucleic acids for use in the invention, as may the progenitors, precursors and stem cells that give rise to the above somatic and germ cells. Also suitable for use as nucleic acid sources are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

Any of the above prokaryotic or eukaryotic cells, tissues and organs may be normal, diseased, transformed, established, progenitors, precursors, fetal or embryonic. Diseased cells may, for example, include those involved in infectious diseases (caused by bacteria, fungi or yeast, viruses (including AIDS, HIV, HTLV, herpes, hepatitis and the like) or parasites), in genetic or biochemical pathologies (e.g., cystic fibrosis, hemophilia, Alzheimer's disease, muscular dystrophy or multiple sclerosis) or in cancerous processes. Transformed or established animal cell lines may include, for example, COS cells, CHO cells, VERO cells, BHK cells, HeLa cells, HepG2 cells, K562 cells, 293 cells, L929 cells, F9 cells, and the like. Other cells, cell lines, tissues, organs and organisms suitable as sources of nucleic acids for use in the present invention will be apparent to one of ordinary skill in the art.

Once the starting cells, tissues, organs or other samples are obtained, nucleic acid molecules (such as mRNA) may be isolated therefrom by methods that are well-known in the art (See, e.g., Maniatis, T., et al., Cell 15:687-701 (1978); Okayama, H., and Berg, P., Mol. Cell. Biol. 2:161-170 (1982); Gubler, U., and Hoffman, B. J., Gene 25:263-269 (1983)). The nucleic acid molecules thus isolated may then be used to prepare cDNA molecules and cDNA libraries in accordance with the present invention.

In the practice of the invention, cDNA molecules or cDNA libraries are produced by mixing one or more nucleic acid molecules obtained as described above, which is preferably one or more mRNA molecules such as a population of mRNA molecules, with a polypeptide having reverse transcriptase activity, under conditions favoring the reverse transcription of the nucleic acid molecule by the action of the enzymes to form one or more cDNA molecules (single-stranded or double-stranded). Such cDNA molecules preferably contain all or a portion of one or more Ter sites.

Methods of the invention may comprise (a) mixing one or more nucleic acid templates (preferably one or more RNA or mRNA templates, such as a population of mRNA molecules) with one or more reverse transcriptases of the invention and (b) incubating the mixture under conditions sufficient to make one or more nucleic acid molecules complementary to all or a portion of the one or more templates. Such methods may include the use of one or more DNA polymerases, one or more nucleotides, one or more primers (e.g., comprising all or a portion of one or more Ter sites), one or more buffers, and the like. The invention may be used in conjunction with methods of cDNA synthesis such as those that are well-known in the art (see, e.g., Gubler, U., and Hoffman, B. J., Gene 25:263-269 (1983); Krug, M. S., and Berger, S. L., Meth. Enzymol. 152:316-325 (1987); Sambrook, J., et al., Molecular Cloning: A Laboratory Manual, 2nd ed., Cold Spring Harbor, N.Y.: Cold Spring Harbor Laboratory Press, pp. 8.60-8.63 (1989); PCT Publication No. WO 99/15702; PCT Publication No. WO 98/47912; and PCT Publication No. WO 98/51699), to produce cDNA molecules or libraries.

Other methods of cDNA synthesis which may advantageously use the present invention will be readily apparent to one of ordinary skill in the art.

Having obtained cDNA molecules or libraries according to the present methods, these cDNAs may be isolated for further analysis or manipulation. Detailed methodologies for purification of cDNAs are taught in the GENETRAPPER™ manual (Invitrogen Corporation (Carlsbad, Calif.)), which is incorporated herein by reference in its entirety, although alternative standard techniques of cDNA isolation that are known in the art (see, e.g., Sambrook, J., et al., Molecular Cloning: A Laboratory Manual, 2nd ed., Cold Spring Harbor, N.Y.: Cold Spring Harbor Laboratory Press, pp. 8.60-8.63 (1989)) may also be used.

In other aspects of the invention, the invention may be used in methods for amplifying nucleic acid molecules. Amplified nucleic acid molecules of the invention preferably contain all or a portion of one or more Ter sites. Nucleic acid amplification methods according to this aspect of the invention may be one-step (e.g., one-step RT-PCR) or two-step (e.g., two-step RT-PCR) reactions. According to the invention, one-step RT-PCR type reactions may be accomplished in one tube thereby lowering the possibility of contamination. Such one-step reactions comprise (a) mixing a nucleic acid template (e.g., mRNA) with one or more reverse transcriptases and with one or more DNA polymerases and (b) incubating the mixture under conditions sufficient to amplify a nucleic acid molecule complementary to all or a portion of the template. Such amplification may be accomplished by the reverse transcriptase activity alone or in combination with the DNA polymerase activity. Two-step RT-PCR reactions may be accomplished in two separate steps. Such a method comprises (a) mixing a nucleic acid template (e.g., mRNA) with a reverse transcriptase, (b) incubating the mixture under conditions sufficient to make a nucleic acid molecule (e.g., a DNA molecule) complementary to all or a portion of the template, (c) mixing the nucleic acid molecule with one or more DNA polymerases and (d) incubating the mixture of step (c) under conditions sufficient to amplify the nucleic acid molecule. For amplification of long nucleic acid molecules (i.e., greater than about 3-5 Kb in length), a combination of DNA polymerases may be used, such as one DNA polymerase having 3′ exonuclease activity and another DNA polymerase being substantially reduced in 3′ exonuclease activity.

Amplification methods which may be used in accordance with the present invention include PCR (U.S. Pat. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822), as well as more complex PCR-based nucleic acid fingerprinting techniques such as Random Amplified Polymorphic DNA (RAPD) analysis (Williams, J. GK., et al., Nucl. Acids Res. 18(22):6531-6535, 1990), Arbitrarily Primed PCR (AP-PCR; Welsh, J., and McClelland, M., Nucl. Acids Res. 18(24):7213-7218, 1990), DNA Amplification Fingerprinting (DAF; Caetano-Anollés et al., Bio/Technology 9:553-557, 1991), microsatellite PCR or Directed Amplification of Minisatellite-region DNA (DAMD; Heath, D. D., et al., Nucl. Acids Res. 21(24): 5782-5785, 1993), and Amplification Fragment Length Polymorphism (AFLP) analysis (EP 0 534 858; Vos, P., et al., Nucl. Acids Res. 23(21):4407-4414, 1995; Lin, J. J., and Kuo, J., FOCUS 17(2):66-70, 1995).

### Supports and Arrays.

Supports for use in accordance with the invention may be any support or matrix suitable for attaching nucleic acid molecules comprising one or more Ter sites or portions thereof and/or molecules comprising all or a portion of a Ter-binding protein of the invention. Supports may be solid supports, semi-solid supports, and/or or any other support known to those skilled in the art. Such molecules may be added or bound (covalently or non-covalently) to the supports of the invention by any technique or any combination of techniques well known in the art.

When non-covalently attached, molecules of the invention may be bound to a support by intramolecular forces well known in the art (e.g., ionic bonds, hydrophobic interactions, Van der Waals forces, hydrogen bonds, etc.) or combinations thereof. Those skilled in the art will appreciate that a support may be derivatized (i.e., given a particular functionality) prior to non-covalent attachment of the molecules of the invention. For example, a support may be derivatized with a charged group to give the support the opposite charge of the molecule of the invention (e.g., the support may be given a positive charge when the molecule of the invention comprises a nucleic acid).

When covalently attached, molecules of the invention (i.e., nucleic acids comprising all or a portion of a Ter site and/or polypeptides comprising all or a portion of a Ter-binding protein) may be attached to a support either directly (i.e., without the use of a linker molecule) or indirectly (i.e., with the use of a linker molecule). Linker molecules, when present, may be of any length and may comprise a variety of reactive functional groups. Linkers may be attached to the molecules of the invention first and subsequently attached to a support. Alternatively, a linker molecule may be attached to a support and the linker-derivatized support reacted with one or more molecules of the invention.

Supports of the invention may comprise silicon, biochips, nitrocellulose, diazocellulose, glass, polystyrene (including microtitre plates), polyvinylchloride, polypropylene, polyethylene, polyvinylidenedifluoride (PVDF), dextran, Sepharose, agar, starch and nylon. Supports of the invention may be in any form or configuration including beads, filters, membranes, sheets, frits, plugs, columns and the like. Supports may also include multi-well tubes (such as microtitre plates) such as 12-well plates, 24-well plates, 48-well plates, 96-well plates, and 384-well plates. Preferred beads are made of glass, latex or a magnetic material (magnetic, paramagnetic or superparamagnetic beads).

Attachment of molecules to supports is well known in the art. For example, U.S. Pat. No. 5,384,261 is directed to a method and device for forming large arrays of polymers on a substrate and is hereby incorporated by reference in its entirety for all it discloses. According to a preferred aspect of the invention, the substrate is contacted by a channel block having channels therein. Selected reagents are flowed through the channels, the substrate is rotated by a rotating stage, and the process is repeated to form arrays of polymers on the substrate. The method may be combined with light-directed methodologies.

U.S. Pat. No. 5,744,305 is another exemplary teaching showing for example, that selectively removable protecting groups allow creation of well defined areas of substrate surface having differing reactivities. The protecting groups can be selectively removed from the surface by applying a specific activator, such as electromagnetic radiation of a specific wavelength and intensity. The specific activator can expose selected areas of surface to remove the protecting groups in the exposed areas.

Protecting groups are used in conjunction with solid phase oligomer syntheses, such as peptide syntheses using natural or unnatural amino acids, nucleotide syntheses using deoxyribonucleic and ribonucleic acids, oligosaccharide syntheses, and the like. In addition to protecting the substrate surface from unwanted reaction, the protecting groups block a reactive end of the monomer to prevent self-polymerization. For instance, attachment of a protecting group to the amino terminus of an activated amino acid, such as an N-hydroxysuccinimide-activated ester of the amino acid, prevents the amino terminus of one monomer from reacting with the activated ester portion of another during peptide synthesis. Alternatively, a protecting group may be attached to the carboxyl group of an amino acid to prevent reaction at this site. Most protecting groups can be attached to either the amino or the carboxyl group of an amino acid, and the nature of the chemical synthesis will dictate which reactive group will require a protecting group. Analogously, attachment of a protecting group to the 5′-hydroxyl group of a nucleoside during synthesis using for example, phosphate-triester coupling chemistry, prevents the 5′-hydroxyl of one nucleoside from reacting with the 3′-activated phosphate-triester of another.

Regardless of specific use, protecting groups are employed to protect a moiety on a molecule from reacting with another reagent. Protecting groups of the present invention have the following characteristics: they prevent selected reagents from modifying the group to which they are attached; they are stable (that is, they remain attached to the molecule) to the synthesis reaction conditions; they are removable under conditions that do not adversely affect the remaining structure; and once removed, do not react appreciably with the surface or surface-bound oligomer. The selection of a suitable protecting group will depend, of course, on the chemical nature of the monomer unit and oligomer, as well as the specific reagents they are to protect against.

Protecting groups are sometimes photoactivatable. The properties and uses of photoreactive protecting compounds have been reviewed. See, McCray et al., Arm. Rev. of Biophys. and Biophys. Chem. (1989) 18:239-270, which is incorporated herein by reference. Photosensitive protecting groups can be removable by radiation in the ultraviolet (UV) or visible portion of the electromagnetic spectrum. Protecting groups can be removable by radiation in the near UV or visible portion of the spectrum. Activation may also be performed by other methods such as localized heating, electron beam lithography, laser pumping, oxidation or reduction with microelectrodes, and the like. Sulfonyl compounds are suitable reactive groups for electron beam lithography. Oxidative or reductive removal is accomplished by exposure of the protecting group to an electric current source, preferably using microelectrodes directed to the predefined regions of the surface which are desired for activation. Other methods may be used in light of this disclosure. Many, although not all, of the photoremovable protecting groups will be aromatic compounds that absorb near-UV and visible radiation. Suitable photoremovable protecting groups are described in, for example, McCray et al., Patchornik, J. Amer. Chem. Soc. (1970) 92:6333, and Amit et al., J. Org. Chem. (1974) 39:192, which are incorporated herein by reference.

In a preferred aspect, methods of the invention may be used to prepare arrays of proteins and/or nucleic acid molecules (RNA or DNA) or arrays of other molecules, compounds, and/or substances. Such arrays may be formed on any matrix or support known in the art (e.g., microplates, glass slides, and/or standard blotting membranes) and may be referred to as microarrays or gene-chips depending on the format and design of the array. Uses for such arrays include gene discovery, gene expression profiling, genotyping (SNP analysis, pharmacogenomics, toxicogenetics), and the preparation of nanotechnology devices.

Synthesis and use of nucleic acid arrays and generally attachment of nucleic acids to supports have been described (see, e.g., U.S. Pat. No. 5,436,327, U.S. Pat. No. 5,800,992, U.S. Pat. No. 5,445,934, U.S. Pat. No. 5,763,170, U.S. Pat. No. 5,599,695 and U.S. Pat. No. 5,837,832). An automated process for attaching various reagents to positionally-defined sites on a substrate is provided in Pirrung, et al. U.S. Pat. No. 5,143,854 and Barrett, et al. U.S. Pat. No. 5,252,743. For example, disulfide-modified oligonucleotides can be covalently attached to supports using disulfide bonds. (See Rogers et al., Anal. Biochem. 266:23-30 (1999).) Further, disulfide-modified oligonucleotides can be peptide nucleic acid (PNA) using solid-phase synthesis. (See Aldrian-Herrada et al., J. Pept. Sci. 4:266-281 (1998).) Thus, nucleic acid molecules comprising one or more Ter sites or portions thereof can be added to one or more supports (or can be added in arrays on such supports).

The attachment of polypeptides to supports is well known in the art. For example, Deutsch, et al., U.S. Pat. No. 4,615,985, describe the attachment of proteins to a nylon support, Ikeda, et al., U.S. Pat. No. 4,582,622, describe the attachment of proteins to magnetic particles, Burton, et al., U.S. Pat. No. 5,998,155, describe the attachment of biotin binding proteins to supports, and Wagner, U.S. Pat. No. 6,120,992, describes the attachment of nucleic acid binding proteins to supports and their subsequent use to bind nucleic acids. The Ter-binding proteins of the present invention may be attached to a support and subsequently used to bind nucleic acid molecules comprising a Ter site.

Essentially, any conceivable support may be employed in the invention. The support may be biological, non-biological, organic, inorganic, or a combination of any of these, existing as particles, strands, precipitates, gels, sheets, tubing, spheres, containers, capillaries, pads, slices, films, plates, slides, etc. The support may have any convenient shape, such as a disc, square, sphere, circle, etc. The support is preferably flat but may take on a variety of alternative surface configurations. For example, the support may contain raised or depressed regions which may be used for synthesis or other reactions. The support and its surface preferably form a rigid support on which to carry out the reactions described herein. The support and its surface are also chosen to provide appropriate light-absorbing characteristics. For instance, the support may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. Other support materials will be readily apparent to those of skill in the art upon review of this disclosure. In a preferred embodiment the support is flat glass or single-crystal silicon.

Thus, the invention provides methods for preparing arrays of nucleic acid molecules of the invention attached to supports. In some embodiments, these nucleic acid molecules will have all or a portion of one or more Ter sites at one or more (e.g., one, two, three or four) positions in the nucleic acid molecule. In some additional embodiments, one nucleic acid molecule may be attached directly to the support, or to a specific section of the support, and one or more additional nucleic acid molecules will be indirectly attached to the support via attachment to the nucleic acid molecule which is attached directly to the support. In such cases, the nucleic acid molecule which is attached directly to the support provides a site of nucleation around which a nucleic acid array may be constructed.

In one aspect, the invention provides supports containing nucleic acid molecules containing Ter sites. In some embodiments, the nucleic acid molecules of these supports will contain at least one Ter site. These bound nucleic acid molecules are useful, for example, for identifying other nucleic acid molecules (e.g., nucleic acid molecules which hybridize to the bound nucleic acid molecules under stringent hybridization conditions) and proteins which have binding affinity for the bound nucleic acid molecules. The Ter sites may be composed of two separate oligonucleotides or may be a single nucleotide in a stem-loop or hairpin configuration. Stem-loop and hairpin oligonucleotides may form a functional Ter site under conditions that permit the hybridization of complementary regions of the oligonucleotide that comprise all or a portion of a Ter site. This will be particularly useful to for the reversible binding of Ter-binding protein containing molecules. The Ter-binding protein containing molecule may be bound to the double stranded portion of the stem-loop or hairpin oligonucleotide comprising all or a portion of the Ter site and then may be eluted from the oligonucleotide by changing the conditions—pH, salt ionic strength, temperature etc.—such that the hybridized portion of the oligonucleotide becomes all or partially single stranded such that the Ter-binding protein no longer binds to the Ter site.

In some embodiments, expression products may also be produced from these bound nucleic acid molecules while the nucleic acid molecules remain bound to the support. Thus, compositions and methods of the invention can be used to identify expression products and products produced by these expression products.

Further, nucleic acid molecules attached to supports may be released from these supports. Methods for releasing nucleic acid molecules include restriction digestion, recombination, and altering conditions (e.g., temperature, salt concentrations, etc.) to induce the dissociation of nucleic acid molecules which have hybridized to bound nucleic acid molecules. Thus, methods of the invention include the use of supports to which nucleic acid molecules have been bound for the isolation of nucleic acid molecules.

Examples of compositions which can be formed by binding nucleic acid molecules to supports are “gene chips,” often referred to in the art as “DNA microarrays” or “genome chips” (see U.S. Pat. Nos. 5,412,087 and 5,889,165, and PCT Publication Nos. WO 97/02357, WO 97/43450, WO 98/20967, WO99/05574, WO 99/05591, and WO99/40105, the disclosures of which are incorporated by reference herein in their entireties). In various embodiments of the invention, these gene chips may contain two- and three-dimensional nucleic acid arrays described herein.

The addressability of nucleic acid arrays of the invention means that molecules or compounds which bind to particular nucleotide sequences can be attached to the arrays. Thus, components such as proteins and other nucleic acids can be attached to specific locations/positions in nucleic acid arrays of the invention.

### Selection Methods

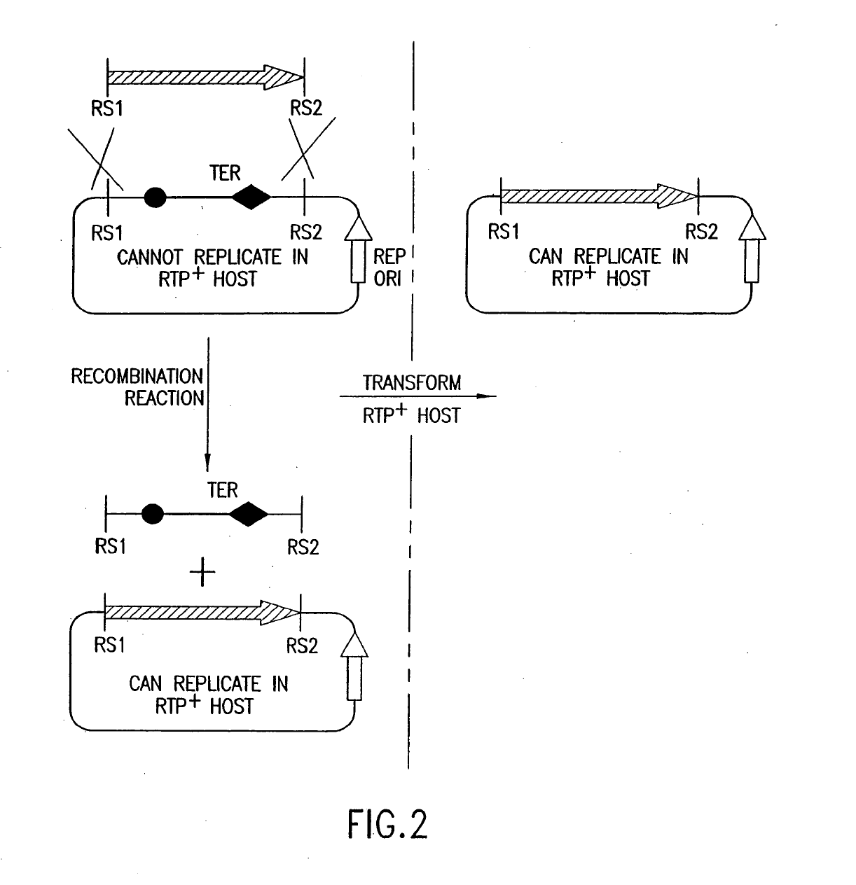

Incorporation of all or a portion of a Ter site into a vector and/or a nucleic acid of interest may permit the selection of desired nucleic acids that either do not contain a Ter site (negative selection) or do contain a sequence of interest (positive selection). With reference to FIG. 2, a vector is prepared comprising a functional Ter site—shown as a darkened circle attached to a darkened diamond. Such a vector may be replicated in a permissive host, i.e., one that does not express an RTP capable of inhibiting the replication of the plasmid. A desired nucleic acid segment—depicted as a striped arrow—is to be inserted into the vector. The vector may optionally comprise recognition sites—restriction sites, topoisomerase sites, recombination sites and the like—to facilitate the insertion and/or removal of nucleic acid segments—for example, RS1 and RS2 in FIG. 2. After conducting one or more reactions—recombination reaction, topoisomerase reactions, and/or digestion and ligation reactions—to insert the segment into the vector a population of molecules is created. In the case of the recombination reaction depicted in FIG. 2, the population includes the desired product as well as unreacted starting vector, and partially reacted vector that includes the insert. Note that the unreacted vector and singly reacted vector both comprise a functional Ter site. When the reaction mixture is transformed into a restrictive host—one that expressed an RTP capable of inhibiting replication of the vector—only those cells that received the desired product—lacking a functional Ter site—can replicate the vector and survive. This is an example of negative selection, i.e., selection against the presence of a Ter site. Negative selection for clones in which the Ter-step has been removed can be enhanced by including a recA mutation in the RTP-expressing host cells. (Hou, et al. Plasmid 47:36-50 (2002)).

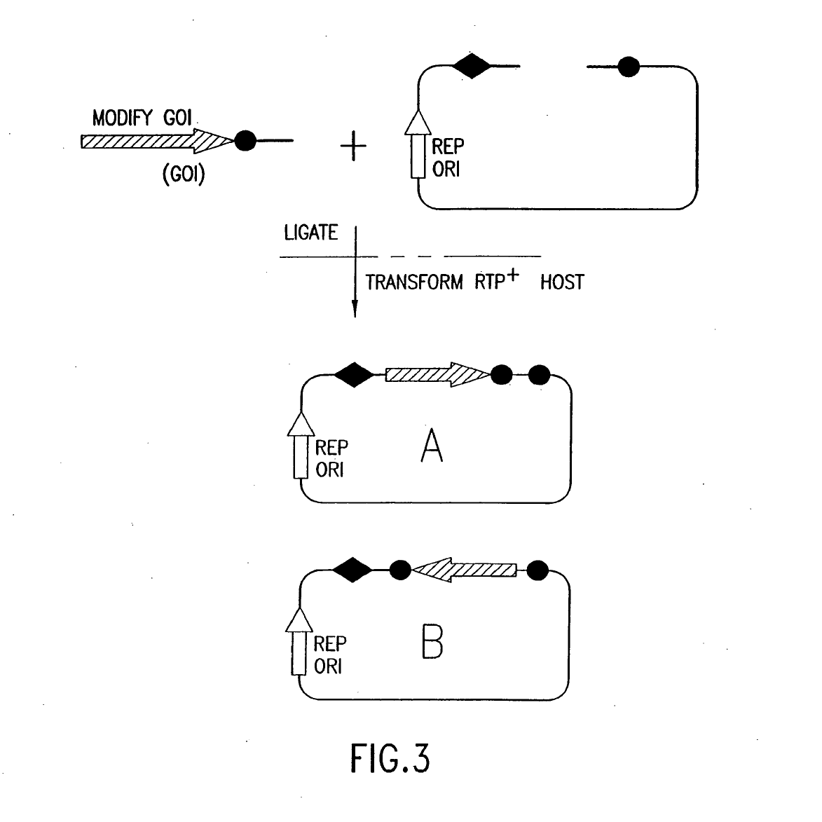

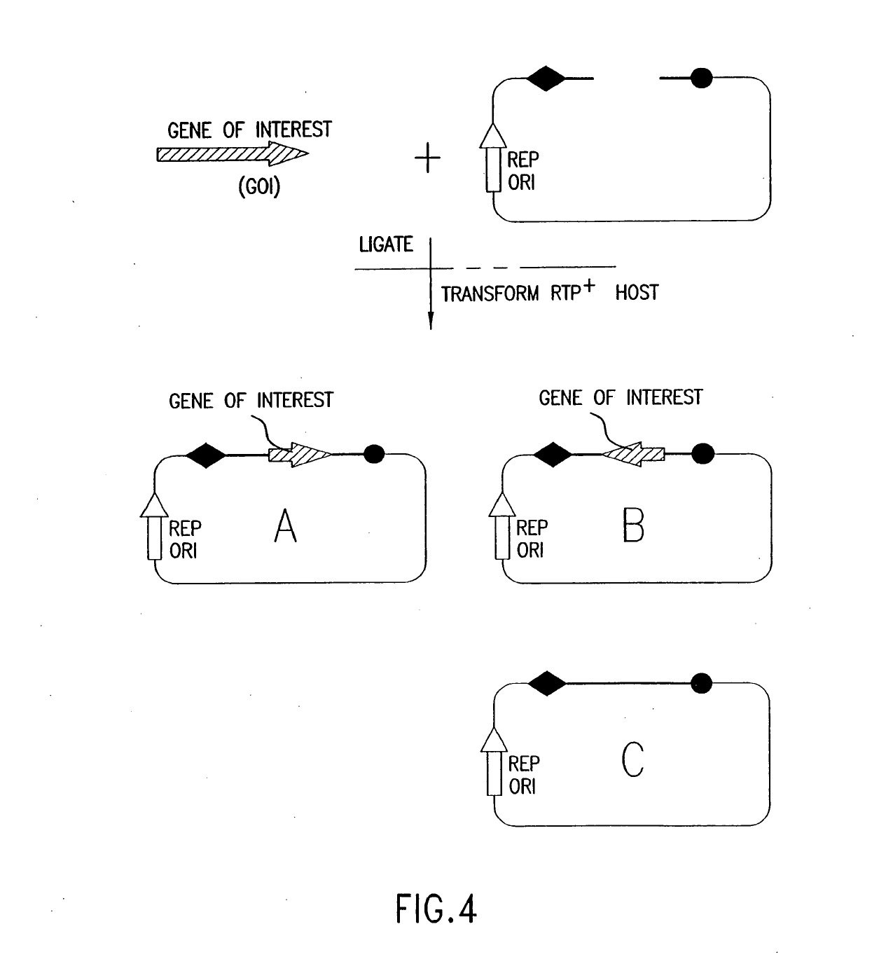

With reference to FIGS. 3 and 4, positive selection for the presence of an insert, optionally in a desired orientation, is shown. In FIG. 3, a gene of interest is modified to comprise a sequence of a portion of a Ter site—depicted as a darkened circle. A vector is prepared comprising the remaining portion of a Ter site. The remaining portion may be provided as an entire Ter site that can be cleaved in the middle—as shown in FIG. 3—or may be provided as just the remaining sequence. The vector is then cleaved so as to generate a linear vector. When the insert is ligated into the vector it may go in in either orientation. In one orientation, a functional Ter site is generated (plasmid B) and in the other, no Ter site is generated (plasmid A). When the reaction mixture is introduced into host cells expressing an RTP, only those cells that receive a vector that does not contain a functional Ter site (plasmid A) can replicate the vector and grow. This is an example of positive selection for a particular orientation of the insert.

With reference to FIG. 4, a vector is prepared that comprises a functional Ter site that can be cleaved. A gene of interest is ligated into cleaved vector and the reaction mixture is used to transform cells expressing an RTP. Only those cells that receive a vector comprising an insert—and hence lacking a Ter site—can replicate (plasmids A and B) in an RTP+ host. This is an example of positive selection for an insert. Plasmids that self-ligate (plasmid C) will not replicate in an RTP⁺ host.

### Detection Methods

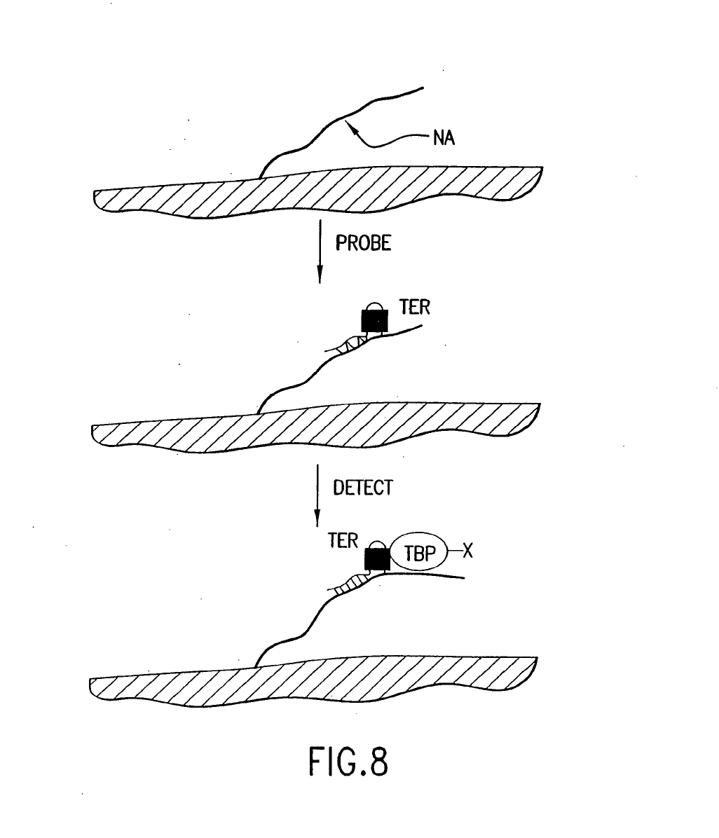

The high affinity of the Ter-binding protein and/or fusion protein comprising a Ter-binding site for the Ter site may advantageously be used to detect molecules comprising a Ter site and/or molecules comprising a Ter-binding protein. Those skilled in the art will appreciate that a detectable molecule may be attached to a molecule comprising a Ter site, to a molecule comprising a Ter-binding protein, or to both. An example of one detection method of the present invention is provided in FIG. 8. A nucleic acid of interest (NA) may be attached to a solid support, for example, as in a Northern or Southern blot. A probe comprising a Ter site (black box) and a sequence that specifically hybridizes to the sequence of interest can be hybridized to the target sequence. The probe may optionally comprise a sequence that forms a stem loop structure and/or a hairpin where the Ter site is contained in the double stranded portion of the probe. Optionally, the probe may contain one strand of a Ter site and an oligonucleotide comprising the other strand may be hybridized to the probe to generate a functional Ter site. After hybridization, the complex comprising the probe and the target sequence is contacted with a Ter-binding protein (TBP). The Ter-binding protein may optionally comprise a detection molecule (X), for example, a fluorophore, chromophore, enzyme or the like. Optionally, the Ter-binding protein may not comprise a detection molecule and may instead be detected using an antibody—optionally labeled—to the Ter-binding protein.

The detection methods of the present invention may be used in a variety of applications including, but not limited to, Southern blots, Northern blots, Western blots, and in situ hybridization.

### Purification Methods

The high affinity of the Ter-binding protein and/or fusion protein comprising a Ter-binding site for the Ter site may advantageously be used in a variety of purification methodologies.

Molecules comprising a Ter site may be contacted in solution by molecules comprising all or a portion of a Ter-binding protein in order to form a binary complex. Optionally, the complex may be contacted with one or more additional molecules to effect isolation. For example, the complex may be contacted with an antibody to the Ter-binding protein to form a ternary complex and the ternary complex may be isolated using standard techniques (e.g.; protein A, protein G, etc.). In some embodiments, the molecule comprising all or a portion of a Ter-binding protein may further comprise one or more functionalities designed to facilitate purification of the binary complex. For example, the molecule comprising all or a portion of the Ter-binding protein may further comprise one or more haptens, ligands and the like.

Molecules comprising nucleic acids comprising a Ter site may be bound, directly or indirectly, to a support and used to bind molecules comprising all or a portion of a Ter-binding protein from a solution. Alternatively, molecules comprising all or a portion of a Ter-binding protein may be attached, directly or indirectly, to a support and used to bind molecules comprising all or a portion of a Ter site.

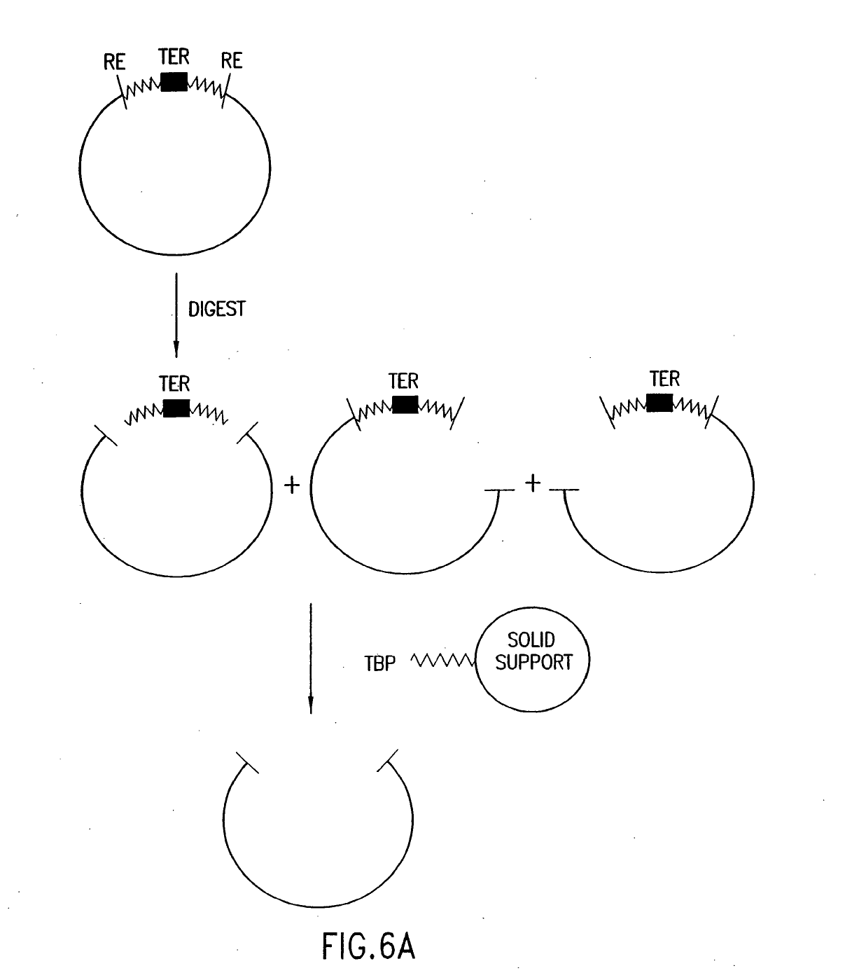

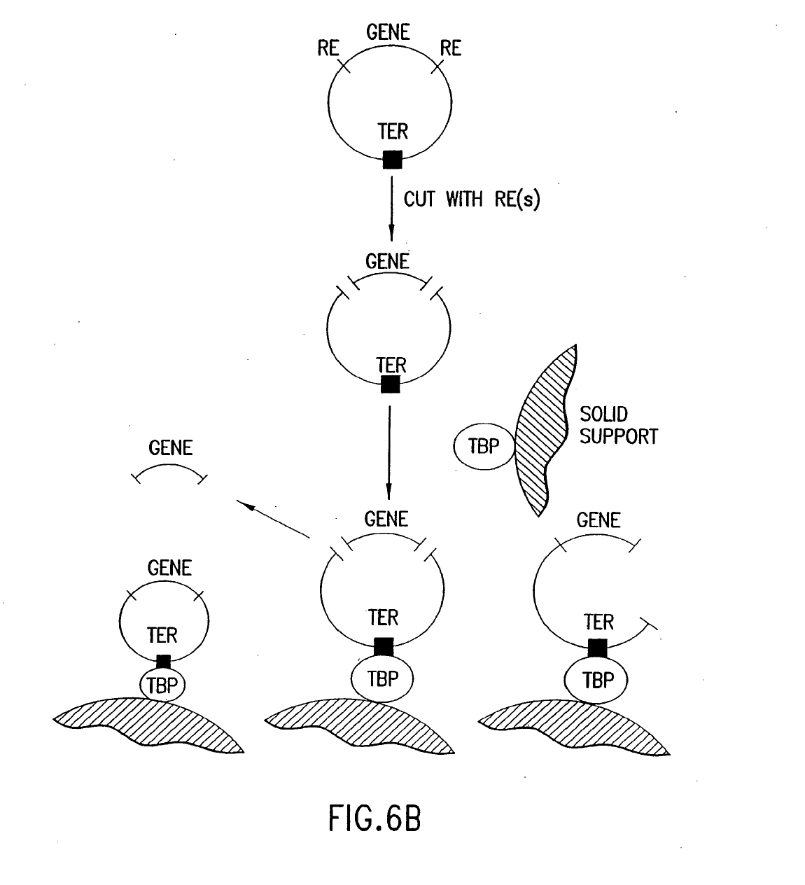

In some embodiments, nucleic acids—for example, plasmids—comprising a Ter site may be used as vectors. In embodiments of this type, the presence of the Ter site in the vector may be used to facilitate the manipulation of the nucleic acid. For example, with reference to FIG. 6A, a nucleic acid comprising a Ter site (black box) on a stuffier fragment (wavy line) of a plasmid may be digested with a restriction enzyme at restriction enzyme sites (RE) and un-digested and partially digested plasmid removed from the reaction mixture by being bound through Ter-binding protein to a solid support. Nucleic acid without Ter sites—correctly digested plasmid in FIG. 6A—are not bound and are thus readily available for further use, such as library construction.

FIG. 6B shows a related aspect in which a vector comprising a Ter site (black box) may contain a sequence of interest—promoter, gene, etc—flanked by restriction and/or recombination sites (RE in FIG. 6B). After the nucleic acid is contacted with the appropriate enzyme—restriction enzyme and/or recombinase—unreacted or partially reacted vector can be removed from solution by contacting the solution with an immobilized protein comprising a Ter-binding site. This facilitates the purification of the product molecule which does not contain a Ter-binding site. The product molecule—i.e., insert—may be subsequently further manipulated as required.

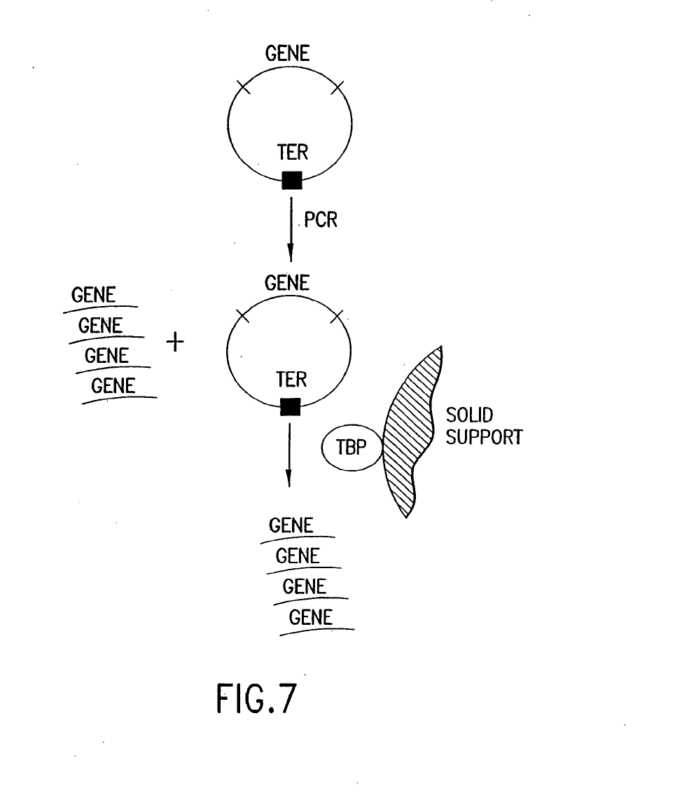

A further embodiment is provided in FIG. 7. In this embodiment, the sequence of interest is amplified or copied from a template comprising a Ter site (black box). The template molecule may be any type of nucleic acid for example, a plasmid or a fragment comprising the sequence of interest. After a sufficient number of copies is prepared, the template molecule may be removed from the reaction mixture by contacting the mixture with an immobilized protein comprising a Ter-binding site (TBP).

Thus, in one aspect, the invention provides affinity purification methods comprising (1) providing a support to which one or more Ter-binding proteins are bound, (2) contacting the support with a composition containing molecules or compounds which have binding affinity for Ter-binding protein bound to the support, under conditions which facilitate binding of the molecules or compounds to the Ter-binding protein bound to the support, (3) altering the conditions to facilitate the release of the bound molecules or compounds, and (4) collecting the released molecules or compounds.

In some embodiments, the present invention provides methods of purifying molecules that comprise all or a portion of a Ter-binding protein. In one embodiment of this type, a fusion protein comprising a Ter-binding protein can be purified by contacting a solution containing the fusion protein with a compound comprising a nucleic acid having a Ter site, for example a magnetic bead to which is attached an oligonucleotide. After binding, the compound—bead—may be washed and the fusion protein eluted.

Thus, in another aspect, the invention provides affinity purification methods comprising (1) providing a support to which nucleic acid molecules comprising at least one Ter site are bound, (2) contacting the support with a composition containing molecules or compounds which have binding affinity for nucleic acid molecules bound to the support, under conditions which facilitate binding of the molecules or compounds to the nucleic acid molecules bound to the support, (3) altering the conditions to facilitate the release of the bound molecules or compounds, and (4) collecting the released molecules or compounds.

### Methods of Manipulating Nucleic Acids

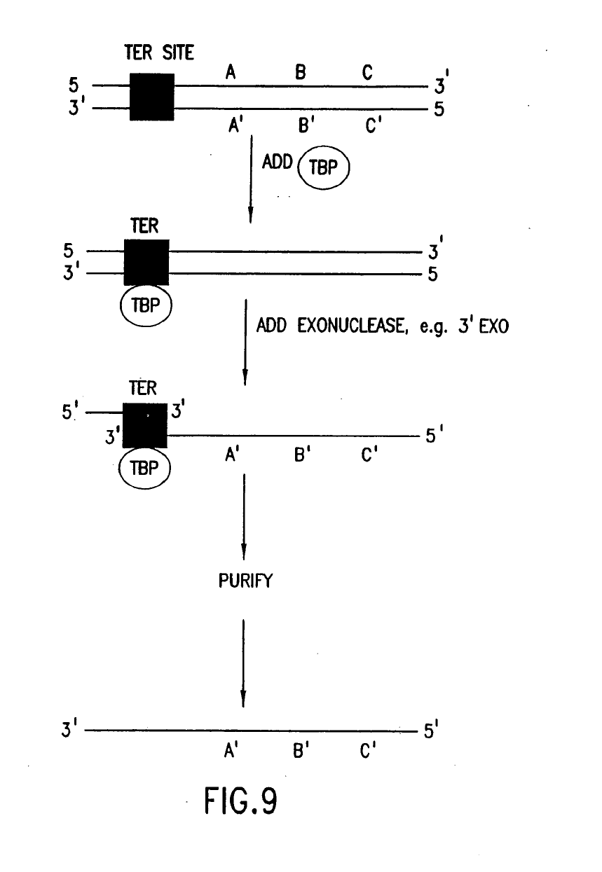

The high affinity of Ter-binding proteins for Ter sites permits various manipulations of nucleic acid molecules that have not been previously possible. For example, with reference to FIG. 9, the affinity of a Ter-binding protein for a Ter site can be used to protect a particular portion of a nucleic acid molecule from, for example, exonuclease digestion. This permits preparation of desired fragments of nucleic acid. In FIG. 9, a fragment of nucleic acid comprising a Ter site (black box) is contacted with a Ter-binding protein (TBP) to form a complex. The fragment is then contacted with an exonuclease, for example a 3′ to 5′ exonuclease. The fragment is digested until the exonuclease reaches the Ter-binding protein where the digestion is halted. This results in the production of a smaller fragment that terminates at the Ter site. As shown in FIG. 9, the Ter-binding protein may be removed and the overlapping portion of the fragment denatured to produce single strands. The single strands may optionally be converted to double strands by hybridizing a primer—for example, one having the sequence of the Ter site—and extending the primer with a polymerase enzyme and nucleoside triphosphates. The result is to produce a smaller fragment having a defined end.

In some embodiments, the present invention provides a method to juxtapose two or more sites in one or more nucleic acid molecules. In its simplest form, a nucleic acid molecule comprising two Ter sites is contacted with a multivalent Ter-binding protein—for example a divalent Ter-binding protein. The multivalent Ter-binding protein binds the nucleic acid at multiple sites thus juxtaposing the sites. In some embodiments, two or more nucleic acids may be juxtaposed. A first nucleic acid comprising a Ter site is contacted with a multivalent Ter-binding protein. The multivalent Ter-binding protein binds the first nucleic acid at the Ter site. The complex of first nucleic acid and Ter-binding protein may optionally be purified from unbound Ter-binding protein and nucleic acid. The complex may then be contacted with a second nucleic acid comprising a Ter site. The multivalent Ter-binding protein then binds the second nucleic acid, thereby juxtaposing the sites. This method may be used to bring sites together for subsequent reactions, for example, ligation and/or recombination reactions.

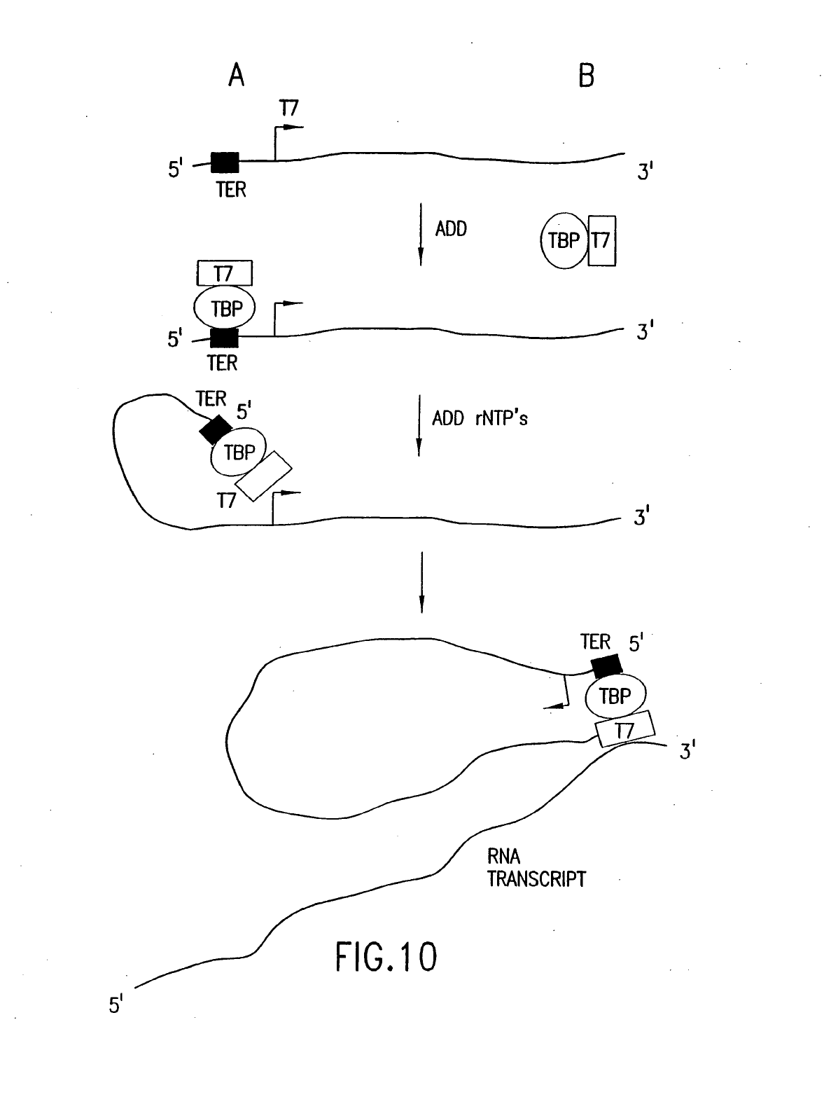

With reference to FIG. 10, two ends of a linear nucleic acid molecule can be brought together using the present invention. A ds DNA contains a Ter site at one end “A” and a promoter for an RNA polymerase (indicated by the arrow and T7) near the Ter site appropriately placed such that DNA/protein interaction and transcription is permitted. The Ter-binding protein (TBP) is functionally associated with the RNA polymerase (T7) that recognizes the promoter, for example, by constructing a fusion protein or chemically coupling a Ter-binding protein to a polymerase. When the Ter-binding protein-RNA polymerase complex is added to the linear ds DNA, the Ter-binding protein binds Ter and RNA polymerase binds the nearby promoter. Addition of nucleotides under certain condition results in transcription by the RNA polymerase which proceeds down the ds DNA toward the other end. The bound Ter-binding protein pulls the “A” end toward the “B” end. The two ends may be annealed or ligated more efficiently when “A” and “B” are in close proximity. Ends of nucleic acid molecules from about 250 base pairs (bp) to 250,000 bp, preferably 1000-100,000 bp can be apposed. Polymerases which could be directed to a specific site on a DNA strand can be used such as E. coli RNA polymerase holoenzyme, T7 RNA polymerase, or SP6 RNA polymerase, to name a few. In this way, intramolecular joining at the ends of a linear DNA may be increased, and formation of chimeric molecules may be decreased.

In addition to its use in cloning, the ability to juxtapose sites in a nucleic acid molecule may be used in the construction and use of nanodevices. The ability of the Ter-binding protein to hold a specific site on a nucleic acid molecule while another protein—for example, a polymerase—pulls the specific site to some distal point on the nucleic acid molecule can be used to move individual strands of a nanodevice as desired.

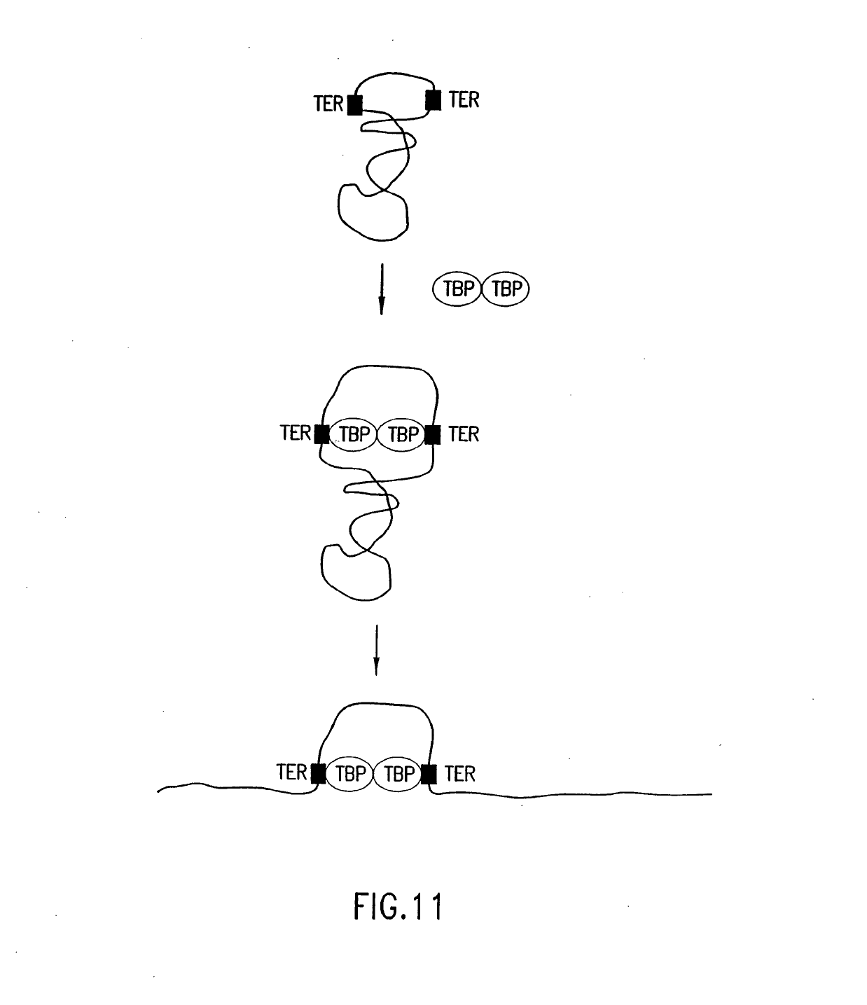

With reference to FIG. 11, the present invention can be used to maintain the topology of a nucleic acid. For example, a supercoiled nucleic acid molecule with two Ter sites (black boxes) may be contacted with a divalent Ter-binding protein (TBP-TBP). The Ter-binding protein holds the nucleic acid rigid, maintaining the topology of the region between the two sites. As exemplified in FIG. 11, the nucleic acid may be optionally cleaved to linearize the molecule; however; the region of the molecule between the Ter sites is maintained in a supercoiled form. In some embodiments, a linear molecule with Ter sites at the ends can be supercoiled by first, contacting the molecule with a divalent Ter-binding protein to bind the two sites and then contacting the molecule with a topoisomerase under conditions causing the super coiling of the nucleic acid molecule. This may be useful for transfection of linear fragments, for example, PCR fragments. Fragments may be prepared with primers incorporating Ter sites. After amplification, the fragments may be contacted with a divalent Ter-binding protein and, subsequently, with a topoisomerase and cofactors, resulting in the production of a supercoiled PCR fragment.

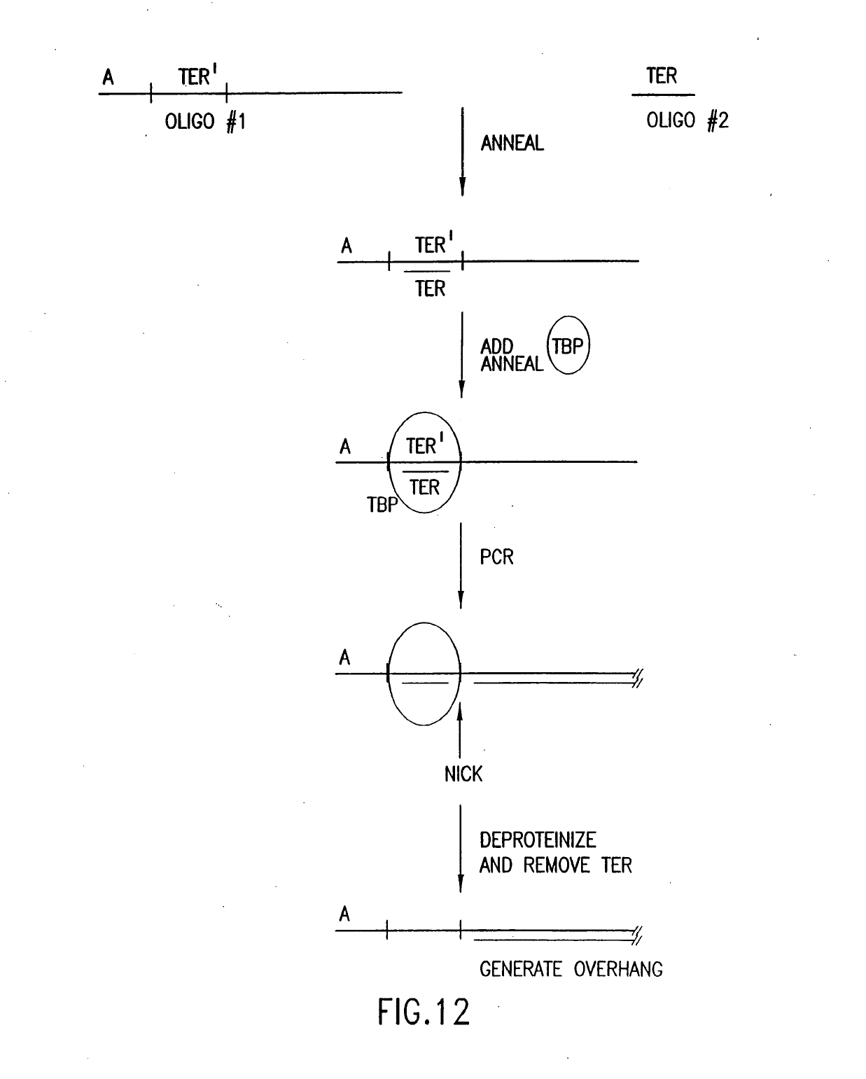

With reference to FIG. 12, the present invention may be used to generate a defined overhang in a nucleic acid molecule comprising a Ter site. A first single stranded nucleic acid comprising one strand of a Ter site is contacted with a second nucleic acid comprising the other strand of the Ter site. After the two strands anneal, a Ter-binding protein is added that binds to the reconstituted Ter site. A primer extension reaction using a primer that anneals to the first nucleic acid at a location 3′ to the Ter site is conducted. The extension is halted at the Ter-binding protein-Ter complex leaving a nick. The Ter-binding protein and the second nucleic acid are removed leaving a defined overhang.

In some embodiments, the present invention provides a method of maintaining a nucleic acid in a duplex under conditions that would normally result in denaturation of the duplex. A nucleic acid comprising one or more Ter sites may be contacted with a Ter-binding protein that recognizes the Ter site. Optionally, the Ter-binding protein may be a thermostable Ter-binding protein. Thermostable Ter-binding proteins may be isolated from thermophilic bacteria or prepared by modifying a Ter-binding protein from a non-thermophilic bacteria. Such modifications include, introducing point mutations in the Ter-binding protein such as introducing cysteine residues to form disulfide bridges, chemically crosslinking the Ter-binding protein using bifunctional crosslinking reagents, cyclizing the Ter-binding protein and the like.

### Kits

In another aspect, the invention provides kits which may be used in conjunction with the invention. Kits according to this aspect of the invention may comprise one or more containers, which may contain one or more components selected from the group consisting of one or more nucleic acid molecules or vectors of the invention, one or more primers, one or more Ter-binding proteins and/or modified Ter-binding proteins of the invention, supports of the invention, one or more polymerases, one or more reverse transcriptases, one or more recombination proteins (or other enzymes for carrying out the methods of the invention), one or more buffers, one or more detergents, one or more restriction endonucleases, one or more nucleotides, one or more terminating agents (e.g., ddNTPs), one or more transfection reagents, one or more host cells that may be competent to take up nucleic acid molecules, pyrophosphatase, one or more proteolytic enzymes and the like. Kits of the invention may comprise one or more written instructions and/or protocols for carrying out the methods of the invention, for making and/or using the nucleic acid molecules and/or proteins of the invention, and/or for making and/or using the compositions and/or reaction mixtures of the invention.

A wide variety of nucleic acid molecules or vectors of the invention can be used with the invention. Further, due to the modularity of the invention, these nucleic acid molecules and vectors can be combined in wide range of ways. Examples of nucleic acid molecules which can be supplied in kits of the invention include those that contain all or a portion of one or more Ter sites and, optionally, one or more promoters, signal peptides, enhancers, repressors, selection markers, transcription signals, translation signals, primer hybridization sites (e.g., for sequencing or PCR), recombination sites, restriction sites and polylinkers, sites which suppress the termination of translation in the presence of a suppressor tRNA, suppressor tRNA coding sequences, sequences which encode domains and/or regions (e.g., 6 His tag) for the preparation of fusion proteins, origins of replication, telomeres, centromeres, and the like. Similarly, libraries can be supplied in kits of the invention. These libraries may be in the form of replicable nucleic acid molecules or they may comprise nucleic acid molecules which are not associated with an origin of replication. As one skilled in the art would recognize, the nucleic acid molecules of libraries, as well as other nucleic acid molecules, which are not associated with an origin of replication either could be inserted into other nucleic acid molecules which have an origin of replication or would be expendable kit components.

Vectors supplied in kits of the invention can vary greatly. In most instances, these vectors will contain an origin of replication, at least one selectable marker, and at least one Ter site and may contain one or more recombination sites. For example, vectors supplied in kits of the invention can have four separate recombination sites which allow for insertion of nucleic acid molecules at two different locations. Other attributes of vectors supplied in kits of the invention are described elsewhere herein.

Kits of the invention may comprise one or more containers containing one or more host cell for use in the practice of the invention. Host cells may be competent to take up nucleic acids (e.g., electrocompetent, chemically competent, etc.). Host cells may be RTP⁺ or RTP⁻. In some instances, kits of the invention may be provided with both RTP⁺ or RTP⁻ cells. Preferred host cells are prokaryotic cells, e.g., E. coli. Examples of preferred host cells include, but are not limited to, DH5, DH5α, TOP10, DH10, DH10B, and other strains available from Invitrogen Corporation, Carlsbad, Calif.

Kits of the invention can also be supplied with primers. These primers will generally be designed to anneal to molecules having specific nucleotide sequences. For example, these primers can be designed for use in PCR to amplify a particular nucleic acid molecule. Further, primers supplied with kits of the invention can be sequencing primers designed to hybridize to vector sequences. Thus, such primers will generally be supplied as part of a kit for sequencing nucleic acid molecules which have been inserted into a vector.

One or more buffers (e.g., one, two, three, four, five, eight, ten, fifteen) may be supplied in kits of the invention. These buffers may be supplied at a working concentrations or may be supplied in concentrated form and then diluted to the working concentrations. These buffers will often contain salt, metal ions, co-factors, metal ion chelating agents, etc. for the enhancement of activities of the stabilization of either the buffer itself or molecules in the buffer. Further, these buffers may be supplied in dried or aqueous forms. When buffers are supplied in a dried form, they will generally be dissolved in water prior to use. Examples of buffers suitable for use in kits of the invention are set out in the following examples.

Supports suitable for use with the invention (e.g., solid supports, semi-solid supports, beads, multi-well tubes, etc., described above in more detail) may also be supplied with kits of the invention.

Kits of the invention may contain virtually any combination of the components set out above or described elsewhere herein. As one skilled in the art would recognize, the components supplied with kits of the invention will vary with the intended use for the kits. Thus, kits may be designed to perform various functions set out in this application and the components of such kits will vary accordingly.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Use of RTP/Ter Interaction in Plasmids

The termination of replication function of the RTP/Ter interaction may be used to select against the presence of Ter sequences in a plasmid. For example, two Ter sequences can be inserted in a particular nucleic acid segment arranged as inverted repeats with the non-permissive side of each Ter site located proximal to the origin of replication. The replication complex will be unable to replicate the segment of the plasmid in between the Ter sites. Thus the plasmid will not be replicated and will be lost. Replication may proceed bi-directionally from the origin until the replication complex reaches the termination sequence. In a host cell which produces a functional RTP, replication of the plasmid would be halted at the Ter sites and the plasmid would not be replicated. In a host cell which does not produce a functional RTP, the plasmid would be replicated.

If desired, the plasmid may comprise one or more additional nucleic acid segments encoding, for example, selectable markers. A selectable marker may be placed at any location on the plasmid including at a location between the Ter sites that is not replicated in a host that produces a functional RTP. The plasmid can be replicated in a RTP− host strain and will not be replicated in a RTP+ strain. The presence of the plasmid may be selected in a RTP− strain using a suitable negative selection such as an antibiotic, for example, when the selectable marker is an antibiotic resistance conferring gene. Other marker genes include, for example, nutritional markers, heavy metals, halogenated organics, osmotic shock, pH shock, temperature shock, post-segregational killing, allele addition, i.e., ccdB, ccdA, restriction gene sets, and conditional lethal sacB.

Another application of a plasmid containing a Ter site is in recombinational cloning methods. For this method, the plasmid may be equipped with recombination sites (RS1 and RS2). A plasmid of this type shown in FIG. 2 may be reacted in a recombination reaction with a nucleic acid comprising recombination sites that react with RS1 and RS2. The result would be replacement of the segment containing the Ter site or sites with a segment from the nucleic acid. Since the resulting molecule would not contain the Ter site(s), it would be replicated in a RTP+ host cell. Any intermediate molecules resulting from the reaction of only one or the other of RS1 and RS2 would still contain Ter site(s) and would not be replicated in a RTP+ host.

### Example 2

### Attachment of Nucleic Acids to Solid Supports

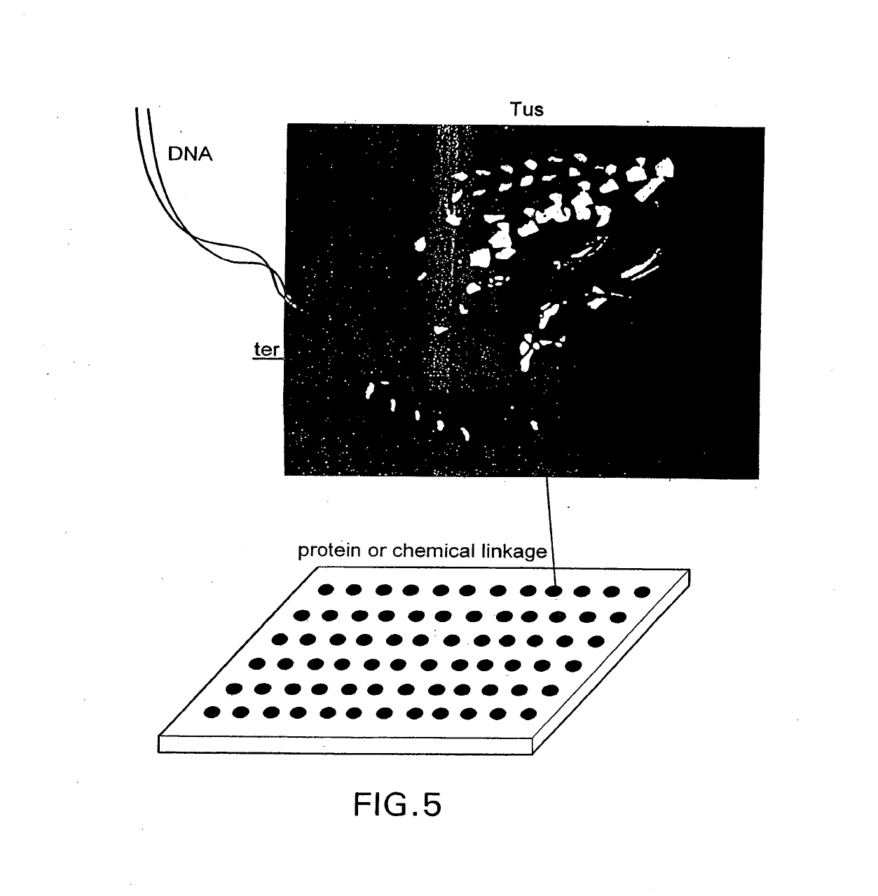

A nucleic acid with a Ter site recognized by a RTP or Ter-binding protein can be attached to a solid support via the Ter-binding protein. For example, a Ter-binding protein may be attached to a solid support by covalent linkage. In some embodiments, reactive groups on the Ter-binding protein may be utilized to attach the protein to a solid support (See FIG. 5). For example, a solid support may be prepared comprising a aldehyde functionality to be coupled to an amine present on the protein. Suitable reagents and techniques for conjugation of the Ter-binding protein to a solid support may be found in Hermanson, Bioconjugate Techniques, Academic Press Inc., San Diego, Calif., 1996. The binding of Ter-binding protein to Ter sites may then be used to attach molecules comprising a Ter site to the solid support.

This methods presents an advantage over standard methods known in the art in that the bound nucleic acids should be more accessible to probes and manipulations because the nucleic acids are attached at one point, not multiple points, as in traditional methods using poly-lysine coated glass for example. Target nucleic acids may also be accessible to a Ter site containing nucleic acid before being introduced into the solid support environment. The Ter-binding protein might then bind a portion or even an entire population of Ter site-containing nucleic acids. Optionally, interaction of the Ter site-containing nucleic acid with a target nucleic acid may be necessary for binding to the Ter-binding protein.

### Example 3

### Directional Cloning of Blunt Ended Fragments

The present invention provides materials and methods for the directional cloning of blunt ended nucleic acid fragments. The blunt ended fragments may be produced by PCR amplification of a nucleic acid target of interest. In some embodiments, an amplification reaction may be performed in which one of the primers used to amplify the DNA target of interest incorporates a sequence corresponding to a portion of a termination sequence. The product of the amplification reaction will be a blunt ended nucleic acid fragment having a portion of a termination sequence at one end. In order to directionally clone such a fragment, the fragment may be ligated into a vector wherein the vector also comprises a portion of a termination site.

In some preferred embodiments, the portion of the termination site contained by the vector and the portion of the termination site contained by the PCR fragment may combine to form one complete termination site (see FIG. 3). In this situation, the blunt-ended fragment may only be cloned into the vector in one direction. The presence of a complete termination site sequence on the resultant plasmid will make the replication of the plasmid extremely inefficient in the presence of replication terminator protein. Since the replication of the host cell into which the plasmid has been inserted is dependent upon the presence of a plasmid encoding a selectable marker, i.e. an antibiotic resistance marker, the replication of host cells containing plasmids in which a complete termination site has been reconstituted will be severely impaired in comparison to those cells in which a termination site was not reconstituted (See FIG. 3).

Thus after ligation two types of vectors will be formed, a vector having a complete termination site sequence and a vector that contains two interrupted portions of a termination site sequence. After transformation two populations of host cells will be formed. One population will comprise a vector containing a complete termination site sequence and the other population will comprise a vector having an interrupted termination site sequence. After growth on a selective media cells containing an interrupted termination sites sequence will grow better than those containing a complete termination sites sequence.

A vector may be constructed so as to introduce a portion of a Ter site adjacent to a recombination site. In some preferred embodiments, the portions of the termination site described above may be combined with all or a portion of a recombination site. In embodiments of this type, insertion of the blunt-ended fragment into the vector will result in the production of a vector that comprises a functional recombination site. After identification of colonies containing the vector having the blunt-ended fragment in the proper orientation, the vectors may be further manipulated using recombinational cloning techniques.

Directional cloning provides for the orientation-specific establishment of a DNA segment of interest into a vector. The fact that the orientation of the fragment is known adds significantly to the value of a given clone construction because the orientation of the segment provides information for subsequent reactions such as what sequencing primer to use and where the open reading frame acid is relative to plasmid-borne expression signals.

In situations where positive selection for recombinants is desired, the gene of interest can be cloned into a vector containing a termination sequence wherein the stuffer fragment disrupts the termination sequence. Replacement of the stuffer by the gene of interest disrupts the termination sequence. Non-recombinant vectors without the stuffer will fail to establish upon transformation into cells since re-ligation of the cloning site without an insert recreates a termination site rendering the plasmid nonreplicable (See FIG. 4). Thus, the direction of the cloned insert and selection for the vector containing the insert may be accomplished in the same step by the same sequence element.

### Example 4

### Preparation of a Selection Vector

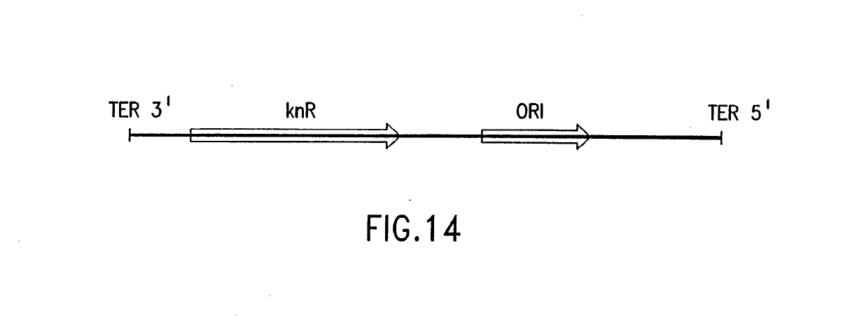

In order to demonstrate the utility of the RTP/Ter interaction in selecting a vector having the insert in the desired orientation, a vector was constructed as follows. The pDONR201 (Invitrogen Corporation, Carlsbad, Calif.) backbone was amplified by PCR using primers that introduced SpeI sites at the core-proximal point of both attL segments. The 5N and 3N sequence of TerB from E. coli were appended to the 5N and 3N ends of the gene for beta-galactosidase using the polymerase chain reaction (PCR). The primers used in PCR introduced restriction enzyme sites allowing for cloning of the amplicon into the aforementioned plasmid backbone, as well as the subsequent removal of beta-galactosidase from the construct. After excision of the beta galactosidase gene, the resulting linear blunt-ended vector was gel purified (FIG. 3 and FIG. 14). The final vector contained an interrupted TerB site after excision of beta-galactosidase. The 5′-end of the TerB site—the diamond and line in FIG. 3—contained nucleotides 1-15 of the TerB sequence in Table 4 while the 3′-end—the circle and line in FIG. 3—contained nucleotides 16-21.

The test insert was constructed using a gene encoding spectinomycin resistance which was amplified by PCR using primers that appended the 3′-portion TerB element to the 3′-end of the spectinomycin gene. The reverse complement of nucleotides 16-21 of the TerB sequence of Table 4 were added to the 3′-end of the spectinomycin gene. In addition, blunt restriction enzyme sites were introduced distal to the 5N expression signals and 3N inverted Ter sequence. The amplicon was digested with these restriction enzymes to yield a blunt fragment.

Ligation: 5 μl of insert DNA was added to either 1 or 10 μl of vector and ligated in a 20 μl reaction for 2.5 h. at 16° C. In addition, either 1 or 10 of vector was subjected to the same reaction conditions without the addition of insert DNA. The reactions were extracted with phenol/chloroform, ethanol precipitated, and reconstituted in 10 μl One hundred μl of library efficiency DH5a (Invitrogen, Carlsbad, Calif.) were transformed with each ligation according to the manufacturer's protocol and plated onto LB with kanamycin.

Two distinct colony morphologies apparent, large and small. The results are shown in Table 15.

TABLE 15

| μl insert | |
|---|---|
| 0 | 5 |

μl vector

| | 1 | 10 | 1 | 10 |
|---|---|---|---|---|
| CFU/100 μl | 0 | 5 | 12 | 95 |

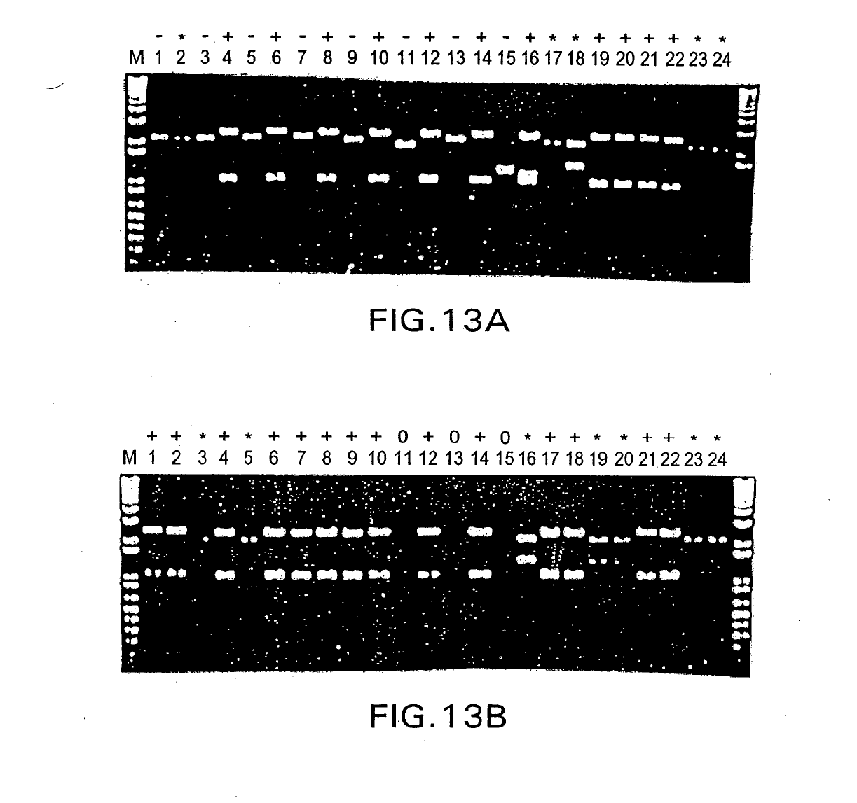

Plasmid DNA was prepared from 8 “no insert” colonies, 12 1:5 (vector:insert ratio) colonies, and 21 10:5 colonies. Both colony morphologies were picked for DNA preparation. DNA was digested with restriction enzymes diagnostic for presence and orientation of insert. Using colony morphology as predictor, 93% (25/27) had desired orientation. Plasmid yield from 83% (10/12) of undesired orientation was comparatively poor, due either to reduced copy number, lower growth rate, or both. (See FIGS. 13A and 13B).

### Example 5

### Improving Transfection Efficiency and Targeting of a Sequence

In another aspect, the present invention provides materials and methods for the improvement of transfection efficiency. In some preferred embodiments, nucleic acids comprising one or more Ter sites may be contacted with a Ter-binding protein in order to improve transfection efficiency and/or expression of a sequence contained on the nucleic acid. In some embodiments, the Ter-binding protein may be modified to comprise one or more modifications that improve cellular uptake, cellular localization, stability of the nucleic acid or combinations thereof. In some embodiments, the Ter-binding protein may be modified so as to comprise one or more ligands recognized by one or more cellular receptors. For example, a Ter-binding protein may be derivatized so as to comprise one or more integrin-binding ligands including, but not limited to, proteins or peptides comprising the amino acid sequence arginine-glycine-aspartic acid (RGD). Such protein or peptides may be part of the primary sequence of a fusion protein between such proteins or peptides and a Ter-binding protein. In other embodiments, such protein or peptides may be attached to a Ter-binding protein using conventional protein-protein linkers. For example, a protein or peptides comprising an RGD sequence via intrinsic amino groups may be linked using a cross-linking reagent such as glutaraldehyde. In other embodiments, a protein or peptide comprising an RGD sequence may be linked to a Ter-binding protein via other reactive functional moieties such as thiol or hydroxyl moieties. Those skilled in the art will appreciate that the linking of reactive functional moieties is routine in the art of protein chemistry.

In some embodiments of this type, a nucleic acid molecule may comprise more than one Ter sites. For example, a linear nucleic acid may have a Ter site on each end of the molecule. The nucleic acid may be contacted with one or more Ter-binding fusion proteins having one or more modifications. In some embodiments, the Ter-binding fusion proteins may comprise two or more different modifications designed to enhance the up take and cellular targeting of the nucleic acid. For example, one Ter-binding fusion protein may be modified to contain a receptor ligand and another to comprise a nuclear localization sequence. The nucleic acid may be contacted with both modified proteins such that one of each type binds to a single nucleic acid molecule. Transfection of the molecule into a cell will be enhanced by the presence of the receptor ligand and expression will be enhanced by the transport of the nucleic acid to the nucleus mediated by the nuclear localization sequence.

### Example 6

### Improve Gene Targeting/Knockouts in Cells Using Ter-Binding Protein/Ter to Protect the Ends of Linear DNA Molecules In Vivo

In some embodiments of the present invention, nucleic acids comprising Ter sites may be contacted with functional Ter-binding proteins and stable nucleic acid-protein complexes may be formed. The stable complexes may then be transfected into a recipient host cell using conventional technologies. Embodiments of this type may be useful to improve the efficiency of gene targeting/knockouts, e.g., for creating knockouts in cells, e.g., embryonic stem cells. In some preferred embodiments, a nucleic acid may be provided with one or more Ter sites that may be on each end of the nucleic acid. When molecules of this type are contacted with Ter-binding proteins and/or Ter-binding fusion proteins, the stable complex may comprise one or more Ter-binding proteins at each end of the nucleic acid. The presence of the Ter-binding protein at the end of the nucleic acid may enhance the stability of the nucleic acid molecule after cellular uptake. A Ter-binding protein for use in embodiments of this type may comprise intracellular targeting sequences, for example nuclear targeting sequences.

In some embodiments, a nucleic acid with two Ter sites may be contacted with a multivalent Ter-binding protein so as to fix the topology of the linear molecule. Optionally, the molecule may be treated to alter the topology by, for example, treating the molecule with one or more topoisomerase enzymes and suitable cofactors.

### Example 7

### Using a Ter-Binding Fusion with a Detection Molecule for Use in the Detection Of Biological Molecules

In some embodiments, the present invention comprises materials and methods for use in the detection of biological molecules. In some embodiments, a Ter-binding protein may comprise a detection molecule. Suitable detection molecules include, but are not limited to, chromophores, fluorophores, enzymes and the like. In some preferred embodiments the detection molecule may be any enzyme whose activity can be measured. Suitable enzymes include, but are not limited to, alkaline phosphatase, beta-galactosidase, beta-glucuronidase and the like. In some embodiments, a Ter-binding protein may comprise multiple detectable moieties which may be the same or different.

In some embodiments, the biological molecule to be detected may be a nucleic acid. In some embodiments, a nucleic acid may be fixed to a solid support such as a filter ad/or an array. In order to detect the nucleic acid of interest, a probe nucleic acid comprising a sequence capable of hybridizing to the nucleic acid of interest may be equipped with a sequence comprising a Ter site. The Ter site may be provided in the form of a hairpin molecule or, alternatively, one strand of a Ter site may be incorporated into the nucleic acid capable of hybridizing to the nucleic acid of interest and a second oligonucleotide having a sequence complementary to the strand of the Ter site incorporated in a nucleic acid may be provided as a separate molecule. In embodiments of this type, the second oligonucleotide may be provided either before or after the hybridization of the probe nucleic acid to the target nucleic acid. After hybridization of the probe molecule comprising a Ter site to the target molecule, the Ter site containing probe molecule may be detected using a Ter-binding protein comprising a detectable portion. This embodiment is exemplified in FIG. 8.

### Example 8

### Using Ter-Binding Protein-Coated Solid Supports

Solid supports to which one or more Ter-binding proteins have been affixed can be used to purify Ter site-containing molecules from a mixture. Mixtures may be the result of conducting a desired reaction, e.g. a PCR reaction. The PCR product or the staring template may comprise a Ter site. After completion of the reaction, the Ter site-containing molecule can be separated from the remainder of the reaction mixture by contacting the mixture with a solid support—for example, magnetic beads—comprising a Ter-binding protein. The remaining components of the mixture can then be washed from the bead and the Ter site-containing molecule eluted from the solid support. This embodiment can be used to separate a variety of biological molecules from mixtures comprising them. Other embodiments include, but are not limited to, separating vectors from inserts; sequencing products from reaction components, DNA from dNTPs or dNMPs, e.g. PCR reactions or exonuclease reactions; plasmids from minipreps, to name a few.

In some embodiments of the present invention, a Ter-binding protein may be covalently attached to one or more solid supports. Solid supports may be of any form customarily used in the art for example, solid supports may be in the form of filters, fibers, membranes, glass slides, beads, and/or 96 well plates.

To purify the nucleic acid with the Ter site, the solution comprising the nucleic acid is brought in contact with the Ter-binding protein attached to the solid support to form a complex. The nucleic acids not containing a Ter site are not bound and can be separated from bound nucleic acid (See FIGS. 6A and 6B). This embodiment will be useful in the purification of plasmids from cellular lysates, for example, in a miniprep.

### Example 9

### Use of Ter-Binding Protein/Ter to Juxtapose Sites in Nucleic Acid Molecules and Increase Synthesis of Product

In yet another aspect, the present invention relates to a method for juxtaposing sites in nucleic acid molecules. In one embodiment, a nucleic acid comprising two Ter sites is contacted with a multivalent—i.e., divalent—Ter-binding protein. Each binding site on the nucleic acid molecule binds to a site on the multivalent Ter-binding protein resulting in the juxtaposition of the two sites (FIG. 11). The nucleic acid may optionally be subjected to additional manipulations, for example, recombination reactions, endonuclease reactions, ligations and the like.

In another embodiment, the present invention can be used to move sites within a molecule into a desired spatial relationship. For example, the present invention can be used to juxtapose two sites—for example—two ends, “A” and “B” of a linear nucleic acid molecule (See FIG. 10). FIG. 10 depicts an embodiment of the invention using an enzyme capable of translocating along a nucleic acid molecule. Although FIG. 10 depicts a polymerase enzyme as the translocation enzyme, those skilled in the art will appreciate that other enzymes, for example, helicases may also be used as translocation enzymes.

The dsDNA contains a Ter site at one end “A” and a promoter for an RNA polymerase near the Ter site appropriately placed such that DNA/protein interaction and transcription is permitted. The Ter-binding protein is functionally associated with the RNA polymerase that recognizes the promoter, for example, by constructing a fusion protein. When the Ter-binding-RNA polymerase complex is added to the linear ds DNA, Ter-binding protein binds Ter and RNA polymerase binds the nearby promoter. Addition of nucleotides under certain condition results in transcription by the RNA polymerase which proceeds down the ds DNA toward the other end. The bound Ter-binding protein pulls the “A” end toward the “B” end. The two ends may be annealed or ligated more efficiently when “A” and “B” are in close proximity. Ends of nucleic acid molecules from about 250 base pairs (bp) to 250,000 bp, preferably 1000-100,000 bp can be apposed. Polymerases which could be directed to a specific site on a DNA strand can be used such as E. coli RNA polymerase holoenzyme, T7 RNA polymerase, or SP6 RNA polymerase, to name a few. In this way, intramolecular joining at the ends of a linear DNA may be increased, and formation of chimeric molecules may be decreased.

Another aspect of embodiments of this type is an increased rate of re-initiation—and hence synthesis of product—that will be observed as a result of the interaction of the Ter-binding protein-polymerase fusion. After completion of synthesis of a first product, the polymerase portion of the fusion protein may release the template molecule. The Ter-binding portion will not release the template resulting in the polymerase being immediately positioned at the promoter where a subsequent round of initiation and polymerization can begin.

### Example 10

### Use of Ter-Binding Proteins to Monitor Production of Single Stranded Nucleic Acids

The inability of Ter-binding proteins to bind to single-stranded Ter sites, can be used to monitor or select for conversion from ds to ss DNA, or vice versa. Monitoring formation of ds DNA can be used to detect formation of ds PCR product, or for real time detection and measurement of formation of double stranded DNA product. For example, amplification of a target sequence may be conducted using a primer that incorporates a Ter sequence. The primer may also comprise a detectable label such as a fluorescent molecule. The amplification may be conducted in the presence of a Ter-binding protein which may optionally comprise a moiety capable of quenching the fluorescence of the detectable label. Since the Ter-binding protein will not bind the primer, the initial fluorescence will not be substantially altered by the Ter-binding protein. As the amplification proceeds, double stranded Ter sites will be formed and bound by the Ter-binding protein. The presence of the quenching moiety on the Ter-binding protein will result in a reduction of the fluorescence.

In another embodiment, an amplification reaction may be conducted using a Ter site-containing primer that will contain both a fluorophore and a quencher arranged so that fluorescence is quenched. A Ter-binding protein, modified to comprise an exonuclease, will be added to the amplification reaction. As amplification proceeds forming double stranded Ter sites, the Ter-binding protein will bind the double stranded sites bringing the exonuclease in position to remove the quencher from the double stranded nucleic acid thereby increasing the observed fluorescence as a function of the formation of double stranded nucleic acid.

In another embodiment, an at least partially single stranded nucleic acid comprising at least a portion Ter site may be bound to a solid support. The bound nucleic acid may be contacted with a second nucleic acid that is also at least partially single stranded and the single stranded portion comprises the a sequence complementary to that of the first nucleic acid such that hybridization of the two nucleic acids results in the formation of a Ter site that may be bound by a Ter-binding protein. The Ter-binding protein may optionally be a modified Ter-binding protein, for example, The Ter-binding protein may comprise a detectable label.

### Example 11

### Use of Ter-Binding Proteins to Produce Single Stranded Nucleic Acids

In yet another aspect, the present invention relates to a method for producing single stranded (ss) DNA from a double-stranded (ds) DNA containing a Ter site (See FIG. 9). The method includes binding a Ter-binding protein to the Ter site on the ds DNA, digesting one strand of DNA with an exonuclease, where the bound Ter-binding protein blocks one strand from digestion with the enzyme, and purifying the remaining undigested ss DNA.

In yet another aspect, the present invention relates to a method for producing a desired fragment. The method includes binding a Ter-binding protein to the Ter site on a ds DNA, digesting one strand of DNA with an exonuclease, where the bound Ter-binding protein blocks one strand from digestion with the enzyme. Optionally, the remaining undigested ss DNA may be purified. This can be used to produce a single stranded (ss) DNA fragment from a double-stranded (ds) DNA containing a Ter site (FIG. 9). Optionally, the ssDNA can be converted to dsDNA.

### Example 12

### Use of Ter-Binding Proteins to Control Topology of a Nucleic Acid

In yet another aspect, the present invention relates to a method for controlling the topology of an nucleic acid molecule. In one aspect, the present invention provides a method to maintain superhelicity of linear DNA where the ds, supercoiled DNA contains two Ter sites one at each end of the segment desired to remain supercoiled after linearization (FIG. 11). A multivalent Ter-binding protein, such as a bivalent Ter-binding protein, is added such that both Ter sites can be bound and result in insulating one topological domain from another such that one domain can rotate independently of the other. Thus, in addition to juxtaposing the two sites as discussed above (Example 9), binding of the divalent Ter-binding protein fixes the topology between the two sites. The bivalent Ter-binding proteins can be made by cloning, with or without linkers, direct repeats of the open reading frame encoding a Ter-binding protein or by crosslinking the two molecules, for example. Once the DNA fragment is linearized, the domain contained by Ter sites remains supercoiled until one of the Ter-binding proteins is released. This method is useful for reactions where supercoiling is beneficial.

In another aspect, a linear nucleic acid molecule with two Ter sites can be supercoiled between the two Ter sites by contacting the linear nucleic acid with a divalent Ter-binding protein to form a complex and contacting the complex with one or more topoisomerase enzymes under conditions resulting in the supercoiling of the molecule.

### Example 13

### Using Ter-Binding Protein/Ter Interaction to Stop a Polymerization Reaction at a Defined Site on a Nucleic Acid Molecule

The presence of a Ter site in a nucleic acid molecule can be used to generate less than full length products in a polymerization reaction, i.e., a PCR reaction or a transcription reaction. For example, a nucleic acid comprising a promoter, for example a T7 promoter, and a Ter site arranged such that transcription from the promoter is directed toward the Ter site, may be contacted with a T7 polymerase and appropriate cofactors. When the nucleic acid has a Ter-binding protein bound to the Ter site, the transcription will proceed until the polymerase is halted by the Ter-binding protein resulting in the production of transcripts of a defined length.

In another aspect, this method may be used to generate a double stranded fragment with a “sticky end” for ease in cloning using PCR. Referring to FIG. 12, an oligonucleotide #1 is generated comprising a single stranded exploitable sequence A, a top strand of duplex Ter site ter′ and a segment capable of annealing to the template. Oligonucleotide #2 comprises a bottom strand of duplex Ter site which hybridizes to terN of oligonucleotide #1.

When oligonucleotide #1 and oligonucleotide #2 are annealed, a complete double stranded Ter site is generated which is attached to a sequence which hybridizes to the desired template. A thermostable Ter-binding protein which recognizes the Ter site is allowed to bind such that the replication fork encountering the complex from the right is halted.

The PCR reaction is started by introducing the template. During PCR, the polymerase is halted at the right side of Ter-binding protein/Ter complex resulting in a nick at that locus.

After PCR, the double stranded DNA is isolated, deproteinized, resulting in the loss of oligonucleotide #2, to generate the desired overhang.

### Example 14

### Methods for Detecting Biological Molecules

In another aspect, the present invention relates to methods for detecting a biological molecule, comprising the steps of contacting a biological molecule with a reagent, the reagent comprising a nucleic acid portion preferably containing at least one Ter site and a portion which forms a specific complex with the biological molecule, contacting the complex with a Ter-binding protein fused to a detection molecule, wherein the Ter-binding protein binds to the nucleic acid portions of the reagent, and detecting the detection molecule, wherein the presence of the detection molecule correlates to the presence of the biological molecule. In some embodiments, the detection molecule may be selected from a group consisting of chromophores, fluorophores, enzymes, and epitopes.

### Example 15

### Simultaneous Cloning of Two Genes into One Vector Using a Single Recombination Reaction

In some embodiments of the present invention, vectors may be constructed that contain one or more Ter sites, optionally flanked by recognition sequences (e.g., recombination sites, restriction enzyme sites, topoisomerase sites, and the like). In some embodiments, the recognition sites may be recombination sites, for example, att sites, lox sites, etc. As discussed above, the presence of one or more Ter sites in a vector may be used to select for vectors that have lost the Ter site and against vectors that contain the site.

Vectors may be constructed that comprise multiple selectable markers, each of which may be flanked by recombination sites. Preferably, the recombination sites flanking a selectable marker do not recombine with each other. The recombination sites flanking one selectable marker may be of the same or different type (e.g., att, lox, etc.) and specificity (e.g., att1, att2, loxP, loxP511, etc.) as those flanking another selectable marker. In some embodiments, the recombination sites flanking one selectable marker are of the same type as those flanking another marker (e.g., both are flanked by att sites) but of different specificities. In a preferred embodiment, a first selectable marker may be flanked by two sites of the same type but having different specificity, for example, an att1 site (e.g., attR1, attL1, attB1, or attP1) and an att2 site (e.g., attR2, attL2, attB2, or attP2), while a second selectable marker may be flanked by two sites of the same type as those flanking the first selectable marker but having a specificity different from each other and different from the sites flanking the first selectable marker, for example, an att5 site (e.g., attR5, attL, attB5, or attP5) and an att11 site (e.g., attR11, attL11, attB11, or attP11).

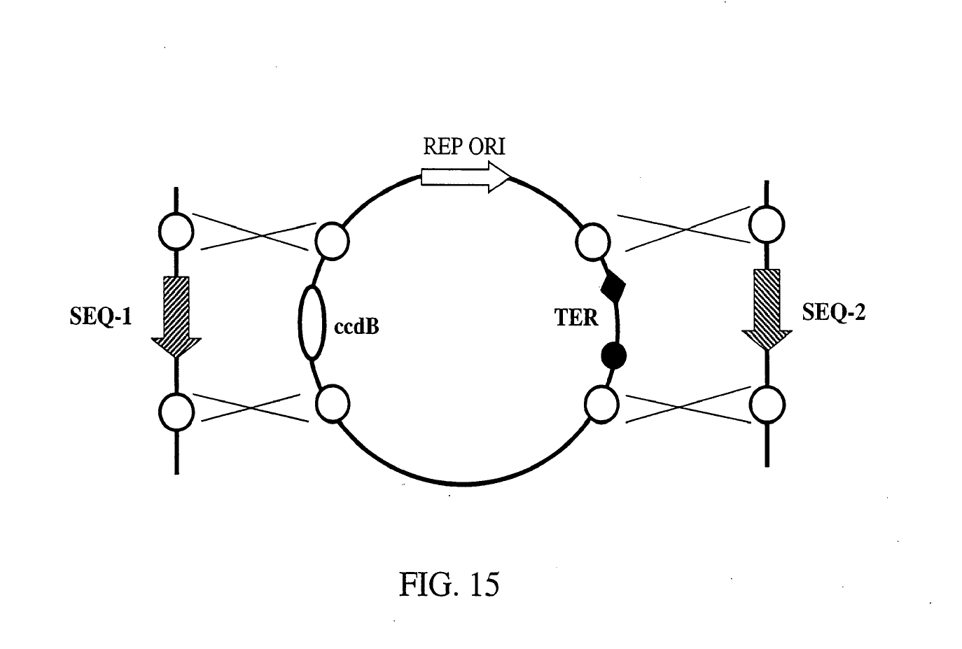

FIG. 15 shows a vector having two different selectable markers (ccdB=oval, and Ter=filled in circle and diamond), each flanked by recombination sites (circles). The vector also comprises an origin of replication (arrow, REP ORI) that directs replication in the direction of the Ter site. Although in FIG. 15 all recombination sites are shown as circles, as discussed above, they may be of the same or different type and/or specificities. In the presence of a nucleic acid molecule having a sequence of interest (SEQ) flanked by the appropriate recombination sites (i.e., those that specifically recombine with the sites in the vector) and the appropriate recombination proteins, a sequence of interest may be inserted into the vector displacing the selectable marker. A sequence of interest may be any type of sequence, for example, may encode an open reading frame (ORF), a gene, a non-translated RNA (e.g., tRNA, RNAi, anti-sense RNA, ribozyme, etc.) or any other sequence known to those skilled in the art. In FIG. 15, the sequences of interest (SEQ-1 and SEQ-2) are depicted as shaded arrows.

Recombination reactions to insert sequences of interest into a vector having multiple selectable markers may be done simultaneously or sequentially. When done sequentially, the vectors having fewer than all of the sequences of interest may be isolated and propagated. Alternatively, sequential insertions of sequences of interest may be done without isolating and propagating the vector between sequential recombination reactions. With reference to FIG. 15, either SEQ-1 or SEQ-2 may be inserted into the vector first and the vector comprising a single sequence may be isolated and propagated. For example, a vector having SEQ-1 inserted in place of the ccdB gene may be propagated in Tus deficient cells; a vector having SEQ-2 inserted in place of the Ter site may be propagated in Tus⁺ cells that are resistant to ccdB (e.g., overexpress ccdA). The vector containing both selectable markers may be propagated in a host cell that overexpresses ccdA and does not express Tus. A vector in which both selectable markers have been replaced by sequences of interest may be expressed in any desired host cell.

In a particular embodiment, vectors containing a Ter site can be used to select for a specific product of a recombination reaction. This is shown in general terms in the embodiment shown in FIG. 2, wherein RS1 and RS2 denote recombination sites. In the scheme shown in FIG. 2, recombination occurs between a DNA fragment containing a sequence of interest (arrow) flanked by recombination sites and a plasmid comprising a Ter site that is oriented so as to block replication of the plasmid. In a cell containing a replication termination protein (e.g., Tus) (RTP⁺), replication of the plasmid is blocked. However, the desired product of the recombination reaction is a plasmid in which the Ter site has been replaced by the sequence of interest. Because it does not comprise the Ter site, the resulting plasmid can replicate in a RTP⁺ cell.

In a preferred embodiment, a site-specific recombination system is used to carry out the recombination reactions. This is shown on the right side of FIG. 15, where the open circles represent sites for a site-specific recombinase. Any appropriate pairing of sites and site-specific recombinases can be used including but not limited to Cre and lox sites, lambda integrase and att sites, etc. A preferred system is the GATEWAY™ system, Invitrogen Corporation, Carlsbad, Calif. Those skilled in the art will be able to position the sites used in a particular site-specific recombination system in the proper location and orientation for any given application of this embodiment.

A vector such as that shown in FIG. 15 may be used to simultaneously clone two sequences of interest into the same vector using a site-specific recombination system. In this embodiment, a toxic gene (e.g., ccdB) is present on the plasmid. The ccdB gene product is toxic to wildtype cells as a result of its interaction with DNA gyrase (Bahassi, et al., J. Biol. Chem. 274 (16):10936-44 (1999). However, the plasmid can be propagated in a host cell that has been altered to be resistant to the effects of ccdB. Examples of host cells that tolerate plasmids comprising ccdB include those that overexpress ccdA or cells that contain a mutant ccdA that is more stable and/or active than the wildtype ccdA gene, or cells that comprise the gyrA462 mutation (Bernard and Couturier, J. Mol. Biol. 226:735-745 (1992)). A preferred E. coli gyrA462 strain is DB3.1™ (Invitrogen Corporation, Carlsbad, Calif.). A Ter site is also present on the plasmid, which prevents the plasmid from replicating in an RTP host cell. In a cell that is deficient in RTP (RTP⁻), however, the plasmid will replicate.

Thus, the vector plasmid shown in FIG. 15 is prepared in a host cell that is ccdB resistant and RTP deficient. The recombination reaction shown on the left side of FIG. 15 yields a product plasmid in which ccdB has been replaced by a sequence of interest (SEQ-1) and which can be propagated in a RTP⁻ cell. The recombination reaction shown on the right side of FIG. 15 results in a product plasmid in which the Ter site has been replaced by a gene of interest (SEQ-2) and which can be propagated in a cell that is resistant to ccdB. When both recombination reactions take place, the resulting product plasmid has neither a ccdB gene nor a Ter site, and can be propagated in a wildtype cell, i.e., a cell that is ccdB-sensitive and RTP⁺.

This “double cloning” method can be used to study the interaction of the proteins encoded by the two cloned genes, and the activities of protein complexes formed thereby. In an exemplary mode, the system is used to study families of proteins that are complexes formed by the combination of two polypeptides, e.g., two leucine zipper proteins. For brevity's sake, a gene encoding a protein comprising a Leucine zipper is called a “Leuzip gene” herein. For example, a first DNA fragment is prepared that encodes a first leucine zipper subunit (Leuzip gene #1) flanked by the appropriate recombination sites needed to effect a recombination reaction that replaces ccdB, and a series of other DNA fragments are prepared that contain other leucine zipper subunits (Leuzip gene #2, Leuzip gene #3, etc.) flanked by sites that effect a recombination reaction with the fragment comprising the Ter site. By way of non-limiting example, the GATEWAY™ system (Invitrogen Corporation, Carlsbad, Calif.) is used. A reaction mix is prepared that contains the vector, a PCR product that comprises Leuzip gene #1 flanked by att sites that specifically react with those on either side of ccdB, and suitable recombination proteins (e.g., Clonase™, Invitrogen Corporation, Carlsbad, Calif.). Aliquots of this reaction mix are prepared, and to each is added a PCR product comprising a PCR product in which att sites that specifically react with the att sites flanking the Ter site flank a different Leuzip gene. Each reaction mix is separately used to transform wildtype cells, and the plasmids in isolated transformants comprise Leuzip gene #1 and the other Leuzip gene added thereto. In this fashion, a series of pairings of different Leuzip genes is generated in a single reaction and transformation.

In addition to being used to study protein complexes, the method can be used to identify pairs of proteins that form complexes having a desired activity. Using leucine zipper proteins as an example, PCR primers comprising att sites are used to amplify a multitude of Leuzip genes from a genome. The PCR products are mixed with the vector plasmid and Clonase, and the mixture is then used to transform wildtype cells. Individual colonies, representing different pairs of Leuzip genes, are isolated and examined for a property or activity of interest. In a screening modality, which may involve high throughput screening (HTS), it may be preferable to directly isolate or identify a clone having the desired activity. For example, a clone expressing a dimeric enzyme having a desired activity on a substrate is identified by placing isolated colonies in wells of a microtitre plate. Radiolabeled substrate is also present in the mixture. In a well containing a cell expressing an enzyme that acts on the substrate, a change in the signal is observed as the substrate is converted into a product compound.

### Example 16

### Construction of Recombinational Cloning Vectors Containing Ter Sites

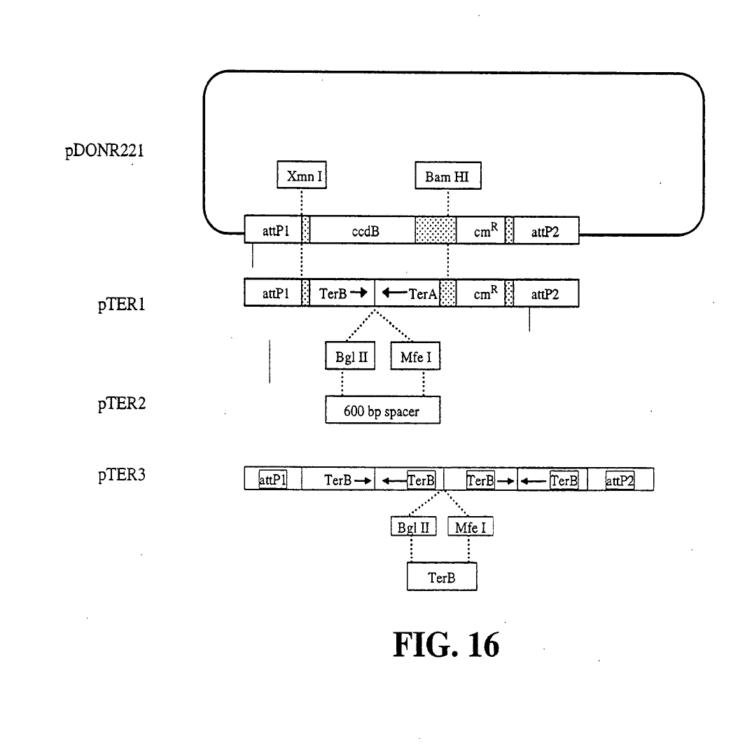

A vector according to the invention may comprise more than one selectable marker arranged in tandem and flanked by recombination sites. When multiple selectable markers are used, the selectable markers may be the same or different. With reference to FIG. 16, three different embodiments having different arrangements of multiple selectable markers are shown. In one embodiment, exemplified by pTER1 in FIG. 16, two different Ter sites (TerA and TerB) are arranged between two recombination sites that do not recombine with each other (attP1 and attP2). A DNA fragment comprising a sequence of interest flanked by attB sites can be recombined with the attP-bounded sequences on pTER1 in order to clone the sequence of interest into the vector. In another embodiment, exemplified by pTER2 in FIG. 16, a vector can be constructed wherein the two Ter sites can be separated by a spacer region of about 600 bp. The spacer may be of any length, for example from 10 bp to about 1 kbp, from about 50 bp to about 750 bp, or from about 100 bp to about 500 bp. In another embodiment, exemplified by pTER3 in FIG. 16, a vector can be construct wherein multiple Ter sites can be arranged in tandem. In embodiments of this type spacers may be inserted between Ter sites and/or between pairs of Ter sites.

The pTER1 vector comprising Ter sites shown in FIG. 16 was constructed as follows. The starting plasmid was pDONR221 (Invitrogen Corporation, Carlsbad, Calif.), which comprises a cassette containing a ccdB gene and a chloramphenical resistance (cm^{r}) gene. The cassette is flanked by two site-specific recombination sites, attP1 and attP2, that are used in the GATEWAY™ system to replace the cassette with a DNA fragment that is flanked by attB on both ends.

The pDONR221 plasmid was digested with the restriction enzymes XmnI and BamHI (FIG. 16). Hybridizing oligonucleotides having internal sequences comprising TerA and TerB and flanking regions having, on one end, sequences that can anneal with the overhang resulting from BamHI (5′-GATC-3′). XmnI does not produce any overhang sequences so no overhang was required at the other end of the molecule formed by the annealed oligonucleotides. The digested plasmid was mixed with the oligonucletoides and ligated together using DNA ligase. The resulting plasmid, pTER1, comprises a cassette flanked by attP sites comprising a TerB and TerA sites arranged in opposing orientations, and a cm^{r} gene. The Ter sites are oriented such that DNA replication forks translocating in either direction will be precluded from proceeding beyond the attP-flanked cassette.

The plasmid pTER2 (FIG. 16) can be generated by digesting pTER1 with BglII and MfeI and ligating into the digested vectlor a ˜600 bp spacer containing a SmaI restriction enzyme site. The ˜600 bp insert is used, for example, in cloning applications where the proximity of a gene to a Ter site might influence expression elements associated with the gene.

The plasmid pTER3 (FIG. 16) can be generated by a scheme similar to that used to create pTER1. That is, pDONR221 may be digested with BamHI and XmnI, and a set of overlapping oligonucleotides may be prepared and ligated into the digested pDONR221. The pTER3 vector will contain four TerB sites, with the junction between the second and third TerB site comprising sites recognized by the restriction enzymes BglII and MfeI. These sites can be used to insert additional Ter sites, spacers and the like into pTER3.

In order to confirm the presence and functionality of Ter sites in these plasmids, the following experiment was carried out. The pTER1 plasmid and a control plasmid (pUC19) were used to transform RTP⁻ and RTP⁺ cells, and the number of transformed colonies was determined. The results are shown in the following Table 16. When Top10 (RTP⁺) cells were transformed with pTER1 and pUC19, transformation with pUC19 DNA yielded over 1,900-fold more cfu/ug (colony-forming units per microgram of DNA) as compared to pTER1. When 838 (RTP⁻) cells were transformed with the two plasmids, transformation with pUC19 DNA yielded only 10-fold more cfu/ug than did pTER1. These data show that a plasmid containing Ter sites aligned so as to block plasmid replication is not viable in RTP+ host cells.

TABLE 16

| Strain | | | | Ratio |
|---|---|---|---|---|
| (Genotype) | | pUC19 | PTER1 | pUC19:pDTER1 |
| TOP10 | (RTP⁺) | 4.8 E8 cfu/ug | 2.5 E5 cfu/ug | 1920x |
| 838 | (RTP⁻) | 2.0 E7 cfu/ug | 1.0 E6 cfu/ug | 10x |

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1. An isolated nucleic acid molecule engineered to comprise all or a portion of at least two Ter sites, wherein the nucleic acid comprises an origin of replication and the Ter sites are arranged with respect to the origin of replication such that the sequence between the two Ter sites is not replicated.

2. The nucleic acid molecule of claim 1, at least one Ter site is selected from a group consisting of TerA, TerB, TerC, TerD, TerE, TerF, TerG, Terh, TerI, and TerJ.

3. The nucleic acid molecule of claim 1, wherein the molecule comprises all or a portion of a TerB site.

4. The nucleic acid molecule according to claim 1, wherein the nucleic acid molecule is selected from a group consisting of plasmids, transposons, BACs, YACs, and phages.

5. The nucleic acid molecule according to claim 1, wherein the molecule is a linear molecule comprising all or a portion of a Ter site capable of being bound by a Ter-binding protein at each end.

6. The molecule according to claim 1, further comprising one or more sequences selected from a group consisting of recombination sequences, restriction enzyme recognition sequences, topoisomerase sites, promoters, enhancers, tag sequences and selectable marker sequences

7. The nucleic acid molecule according to claim 6, wherein the recombination site is a site specific recombination site.

8. The nucleic acid molecule according to claim 7, wherein the recombination site is an att site.

9. The nucleic acid molecule according to claim 8, wherein the att site comprises a sequence of Table 3.

10. A support comprising the engineered isolated nucleic acid molecule of claim 1 coupled thereto.

11. The support according to claim 10, wherein the support is a non-biological material.

12. The support according to claim 10, wherein the oligonucleotide is capable of forming a stem-loop or hairpin.

13. The support according to claim 10, wherein a duplex portion of a stem-loop or hairpin comprises all or a portion of a Ter site.

14. A support comprising all or a portion of a Ter-binding protein.

15. The support according to claim 14, wherein solid support is a non-biological material.

16. The support according to claim 14, wherein the Ter-binding protein comprises all or a portion of one or more sequences selected from the group of sequences of Tables 5-14.

17-28. (canceled)


SEQUENCE LISTING (87 sequences)

SEQ ID NO: 1 - DNA, 23 bp, Escherichia coli
aattagtatgttgtaactaaagt

SEQ ID NO: 2 - DNA, 23 bp, Escherichia coli
aataagtatgttgtaactaaagt

SEQ ID NO: 3 - DNA, 23 bp, Escherichia coli
atataggatgttgtaactaatat

SEQ ID NO: 4 - DNA, 23 bp, Escherichia coli
cattagtatgttgtaactaaatg

SEQ ID NO: 5 - DNA, 21 bp, Escherichia coli
ttaaagtatgttgtaactaag

SEQ ID NO: 6 - DNA, 23 bp, Escherichia coli
ccttcgtatgttgtaacgacgat

SEQ ID NO: 7 - DNA, 23 bp, Escherichia coli
gatgagtatgttgtaactaacta

SEQ ID NO: 8 - DNA, 23 bp, Salmonella typhimurium
attaagtatgttgtaactaaagc

SEQ ID NO: 9 - DNA, 23 bp, Salmonella typhimurium
gatgagtatgttgtaactaaatg

SEQ ID NO: 10 - DNA, 23 bp, Artificial Sequence
  Plasmid R6KterR1
ctcttgtgtgttgtaactaaatc

SEQ ID NO: 11 - DNA, 23 bp, Artificial Sequence
  Plasmid R6KterR2
ctattgagtgttgtaactactag

SEQ ID NO: 12 - DNA, 23 bp, Artificial Sequence
  Plasmid R100TerR1
attatgaatgttgtaactacttc

SEQ ID NO: 13 - DNA, 23 bp, Artificial Sequence
  Plasmid R100TerR2
tgtctgagtgttgtaactaaagc

SEQ ID NO: 14 - DNA, 23 bp, Artificial Sequence
  Plasmid R1TerR1
attatgaatgttgtaactacatc

SEQ ID NO: 15 - DNA, 23 bp, Artificial Sequence
  Plasmid R1TerR2
tttttgtgtgttgtaactaaatt

SEQ ID NO: 16 - DNA, 23 bp, Artificial Sequence
  Plasmid RepFICTerR1
attatgaatgttgtaactacatt

SEQ ID NO: 17 - DNA, 23 bp, Artificial Sequence
  St90kbTer
attttggatgttgtaactatttg

SEQ ID NO: 18 - DNA, 30 bp, Bacillus atrophaeus
gaactaaataaactatgtaccaaatgttca

SEQ ID NO: 19 - DNA, 30 bp, Bacillus atrophaeus
taactgaaaacactatgtactaaatattca

SEQ ID NO: 20 - DNA, 30 bp, Bacillus mojavensis
gaacaaaacaaactatgtaccaaatgttca

SEQ ID NO: 21 - DNA, 30 bp, Bacillus mojavensis
aaactgagaatactatgtactaaatattca

SEQ ID NO: 22 - DNA, 30 bp, Bacillus vallismortis
atactaaaaatatgatgtactaaatattca

SEQ ID NO: 23 - DNA, 30 bp, Bacillus amyloliquefaciens
taacaaattattccatgtactaaatattct

SEQ ID NO: 24 - DNA, 30 bp, Bacillus subtilis 168
gaactaattaaactatgtactaaattttca

SEQ ID NO: 25 - DNA, 30 bp, Bacillus subtilis 168
atactaattgatccatgtactaaattttca

SEQ ID NO: 26 - DNA, 15 bp, Artificial Sequence
  Core Region of the Wildtype att site
gcttttttatactaa

SEQ ID NO: 27 - DNA, 21 bp, Artificial Sequence
  Core sequence of att site
caacttttttatacaaagttg

SEQ ID NO: 28 - DNA, 25 bp, Artificial Sequence
  mutated attB1 site
agcctgcttttttgtacaaacttgt

SEQ ID NO: 29 - DNA, 233 bp, Artificial Sequence
  Mutated attP1 site
tacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaagttggcattataaaaaagcattgctcatcaatttgttgcaacgaacaggtcactatcagtcaaaataaaatcattatttg

SEQ ID NO: 30 - DNA, 100 bp, Artificial Sequence
  Mutated attL1 site
caaataatgattttattttgactgatagtgacctgttcgttgcaacaaattgataagcaatgcttttttataatgccaactttgtacaaaaaagcaggct

SEQ ID NO: 31 - DNA, 125 bp, Artificial Sequence
  Mutated attR1 site
acaagtttgtacaaaaaagctgaacgagaaacgtaaaatgatataaatatcaatatattaaattagattttgcataaaaaacagactacataatactgtaaaacacaacatatccagtcactatg

SEQ ID NO: 32 - DNA, 27 bp, Artificial Sequence
  Wild type attB0 site
agcctgcttttttatactaacttgagc

SEQ ID NO: 33 - DNA, 27 bp, Artificial Sequence
  Wild type attP0 site
gttcagcttttttatactaagttggca

SEQ ID NO: 34 - DNA, 27 bp, Artificial Sequence
  Wild type attL0 site
agcctgcttttttatactaagttggca

SEQ ID NO: 35 - DNA, 27 bp, Artificial Sequence
  Wild type attR0 site
gttcagcttttttatactaacttgagc

SEQ ID NO: 36 - DNA, 25 bp, Artificial Sequence
  Mutated attB1 site
agcctgcttttttgtacaaacttgt

SEQ ID NO: 37 - DNA, 27 bp, Artificial Sequence
  Mutated attP1 site
gttcagcttttttgtacaaagttggca

SEQ ID NO: 38 - DNA, 27 bp, Artificial Sequence
  Mutated attL1 site
agcctgcttttttgtacaaagttggca

SEQ ID NO: 39 - DNA, 25 bp, Artificial Sequence
  Mutated attR1 site
gttcagcttttttgtacaaacttgt

SEQ ID NO: 40 - DNA, 25 bp, Artificial Sequence
  Mutated attB2 site
acccagctttcttgtacaaagtggt

SEQ ID NO: 41 - DNA, 27 bp, Artificial Sequence
  Mutated attP2 site
gttcagctttcttgtacaaagttggca

SEQ ID NO: 42 - DNA, 27 bp, Artificial Sequence
  Mutated attL2 site
acccagctttcttgtacaaagttggca

SEQ ID NO: 43 - DNA, 25 bp, Artificial Sequence
  Mutated attR2 site
gttcagctttcttgtacaaagtggt

SEQ ID NO: 44 - DNA, 22 bp, Artificial Sequence
  Mutated attB5 site
caactttattatacaaagttgt

SEQ ID NO: 45 - DNA, 27 bp, Artificial Sequence
  Mutated attP5 site
gttcaactttattatacaaagttggca

SEQ ID NO: 46 - DNA, 24 bp, Artificial Sequence
  Mutated attL5 site
caactttattatacaaagttggca

SEQ ID NO: 47 - DNA, 25 bp, Artificial Sequence
  Mutated attR5 site
gttcaactttattatacaaagttgt

SEQ ID NO: 48 - DNA, 22 bp, Artificial Sequence
  Mutated attB11 site
caacttttctatacaaagttgt

SEQ ID NO: 49 - DNA, 27 bp, Artificial Sequence
  Mutated attP11 site
gttcaacttttctatacaaagttggca

SEQ ID NO: 50 - DNA, 24 bp, Artificial Sequence
  Mutated attL11 site
caacttttctatacaaagttggca

SEQ ID NO: 51 - DNA, 25 bp, Artificial Sequence
  Mutated attR11 site
gttcaacttttctatacaaagttgt

SEQ ID NO: 52 - DNA, 22 bp, Artificial Sequence
  Mutated attB17 site
caacttttgtatacaaagttgt

SEQ ID NO: 53 - DNA, 27 bp, Artificial Sequence
  Mutated attP17 site
gttcaacttttgtatacaaagttggca

SEQ ID NO: 54 - DNA, 24 bp, Artificial Sequence
  Mutated attL17 site
caacttttgtatacaaagttggca

SEQ ID NO: 55 - DNA, 25 bp, Artificial Sequence
  Mutated attR17 site
gttcaacttttgtatacaaagttgt

SEQ ID NO: 56 - DNA, 22 bp, Artificial Sequence
  Mutated attB19 site
caactttttcgtacaaagttgt

SEQ ID NO: 57 - DNA, 27 bp, Artificial Sequence
  Mutated attP19 site
gttcaactttttcgtacaaagttggca

SEQ ID NO: 58 - DNA, 24 bp, Artificial Sequence
  Mutated attL19 site
caactttttcgtacaaagttggca

SEQ ID NO: 59 - DNA, 25 bp, Artificial Sequence
  Mutated attR19 site
gttcaactttttcgtacaaagttgt

SEQ ID NO: 60 - DNA, 22 bp, Artificial Sequence
  Mutated attB20 site
caactttttggtacaaagttgt

SEQ ID NO: 61 - DNA, 27 bp, Artificial Sequence
  Mutated attP20 site
gttcaactttttggtacaaagttggca

SEQ ID NO: 62 - DNA, 24 bp, Artificial Sequence
  Mutated attL20 site
caactttttggtacaaagttggca

SEQ ID NO: 63 - DNA, 25 bp, Artificial Sequence
  Mutated attR20 site
gttcaactttttggtacaaagttgt

SEQ ID NO: 64 - DNA, 22 bp, Artificial Sequence
  Mutated attB21 site
caactttttaatacaaagttgt

SEQ ID NO: 65 - DNA, 27 bp, Artificial Sequence
  Mutated attP21 site
gttcaactttttaatacaaagttggca

SEQ ID NO: 66 - DNA, 24 bp, Artificial Sequence
  Mutated attL21 site
caactttttaatacaaagttggca

SEQ ID NO: 67 - DNA, 25 bp, Artificial Sequence
  Mutated attR21 site
gttcaactttttaatacaaagttgt

SEQ ID NO: 68 - DNA, 23 bp, Escherichia coli
cgatcgtatgttgtaactatctc

SEQ ID NO: 69 - DNA, 23 bp, Escherichia coli
aacatgtatgttgtaactaaccg

SEQ ID NO: 70 - DNA, 23 bp, Escherichia coli
acgcagtaagttgtaactaatgc

SEQ ID NO: 71 - protein, 309 aa, Escherichia coli [sequence omitted]

SEQ ID NO: 72 - protein, 309 aa, Escherichia coli
MARYDLVDRLNTTFRQMEQELAAFAAHLEQHKLLVARVFSLPEVKKEDEHNPLNRIEVKQHLGNDAQSQALRHFRHLFIQQQSENRSSKAAVRLPGVLCYQVDNLSQAALVSHIQHINKLKTTFEHIVTVESELPTAARFEWVHRHLPGLITLNAYRTLTVLHDPATLRFGWANKHIIKNLHRDEVLAQLEKSLKSPRSVAPWTREEWQRKLEREYQDIAALPQNAKLKIKRPVKVQPIARVWYKGDQKQVQHACPTPLIALINRDNGAGVPDVGELLNYDADNVQHRYKPQAQPLRLIIPRLHLYVAD

SEQ ID NO: 73 - protein, 309 aa, Salmonella typhimurium
MSRYDLVERLNGTFRQIEQHLAALTDNLQQHSLLIARVFSLPQVTKEAEHAPLDTIEVTQHLGKEAEALALRHYRHLFIQQQSENRSSKAAVRLPGVLCYQVDNATQLDLENQIQRINQLKTTFEQMVTVESGLPSAARFEWVHRHLPGLITLNAYRTLTLINNPATIRFGWANKHIIKNLSRDEVLSQLKKSLASPRSVPPWTREQWQFKLEREYQDIAALPQQARLKIKRPVKVQPISRIWYKGQQKQVQHACPTPIIALINTDNGAGVPDIGGLENYDADNIQHRFKPQAQPLRLIIPRLHLYVAD

SEQ ID NO: 74 - protein, 309 aa, Salmonella typhi
MSRYDLVERLNGTFRQIEQHLAALSDNLQQHSLLIASVFSLPQVTKEAEHAPLDTIEVTQHLGKEAEALALRHYRHLFIQQQSENRSSKAAVRLPGVLCYQVDNATQLDLENQVQRINQLKTTFEQMVTVESGLPSAARFEWVHRHLPGLITLNAYRTLTLINNPATIRFGWANKHIIKNLSRDEVLSQLKKSLASPRSVPPWTREQWQFKLEREYQDIAALPQQAKLKIKRPVKVQPIARIWYKGQQKQVQHACPSPIIALINTDNGAGVPDIGGLENYDADNIQHRFKPQAQPLRLIIPRLHLYVAD

SEQ ID NO: 75 - protein, 309 aa, Salmonella enterica; identical to SEQ ID NO: 74

SEQ ID NO: 76 - protein, 310 aa, Klebsiella pneumoniae
MASYDLVERLNNTFRQIELELQALQQALSDCRLLAGRVFELPAIGKDAEHDPLATIPVVQHIGKTALARALRHYSHLFIQQQSENRSSKAAVRLPGAICLQVTAAEQQDLLARIQHINALKATFEKIVTVDSGLPPTARFEWVHRHLPGLITLSAYRTLTPLVDPSTIRFGWANKHVIKNLTRDQVLMMLEKSLQAPRAVPPWTREQWQSKLEREYQDIAALPQRARLKIKRPVKVQPIARVWYAGEQKQVQYACPSPLIALMSGSRGVSVPDIGELLNYDADNVQYRYKPEAQSLRLLIPRLHLWLASE

SEQ ID NO: 77 - protein, 294 aa, Proteus vulgaris
MDLKKTFEQLTDDLLALKMLISGSSPLFSQVSDIPPVLRGDEHLPISYVAPDHLYGHEAIQKAVDIWSDLHIKHDFSQKSARRASGVLWFPSEDNAFTVELVRLLSQINALKKSIETHIITTYQTRSARFEALHNQCAGVLTLHLYRQIRWWKDEHISAVRFSWQEKESLLIPDKAELLVRMSKEGREDGKKEVPLALLMKQIVSVPEERLRIRRRLKVQPSANISFRSEQHPTGKLTMVTAPMPFIIIQNERPEVKMLKIYDANERISRKRRNDKVHTEILGTFHGESIEVIA

SEQ ID NO: 78 - protein, 122 aa, Bacillus subtilis
MKEEKRSSTGFLVKQRAFLKLYMITMTEQERLYGLKLLEVLRSEFKEIGFKPNHTEVYRSLHELLDDGILKQIKVKKEGAKLQEVVLYQFKDYEAAKLYKKQLKVELDRCKKLIEKALSDNF

SEQ ID NO: 79 - protein, 311 aa, Yersinia pestis
MNKYDLIERMNTRFAELEVTLHQLHQQLDDLPLIAARVFSLPEIEKGTEHQPIEQITVNITEGEHAKKLGLQHFQRLFLHHQGQHVSSKAALRLPGVLCFSVTDKELIECQDIIKKTNQLKAELEHIITVESGLPSEQRFEFVHTHLHGLITLNTYRTITPLINPSSVRFGWANKHIIKNVTREDILLQLEKSLNAGRAVPPFTREQWRELISLEINDVQRLPEKTRLKIKRPVKVQPIARVWYQEQQKQVQHPCPMPLIAFCQHQLGAELPKLGELTDYDVKHIKHKYKPDAKPLRLLVPRLHLYVELEP

SEQ ID NO: 80 - protein, 294 aa, Artificial Sequence; identical to SEQ ID NO: 77
  IncT plasmid R394 Ter-binding protein

SEQ ID NO: 81 - protein, 7 aa, Artificial Sequence
  nuclear localization sequence
PKKKRKV

SEQ ID NO: 82 - protein, 10 aa, Influenza virus
AAFEDLRVLS

SEQ ID NO: 83 - protein, 5 aa, Adenovirus
KRPRP

SEQ ID NO: 84 - protein, 5 aa, Artificial Sequence
  lysosomal targeting sequence
KFERQ

SEQ ID NO: 85 - protein, 16 aa, Artificial Sequence
  mitochondrial targeting sequence
MLSLRQSIRFFKPATR

SEQ ID NO: 86 - protein, 4 aa, Artificial Sequence
  Factor Xa cleavage site
IEGR

SEQ ID NO: 87 - protein, 4 aa, Artificial Sequence
  thrombin cleavage site
LVPR